(19) United States
(12) Patent Application Publication
(10) Pub. No.: US 2015/0171346 A1
(43) Pub. Date: Jun. 18, 2015

(54) Novel Organic Electroluminescent Compounds and Organic Electroluminescent Device Using The Same

(71) Applicant: Rohm and Haas Electronic Materials Korea Ltd., Chungcheongnam-do (KR)

(72) Inventors: Hee-Choon Ahn, Gyeonggi-do (KR); Seok-Keun Yoon, Gyeonggi-do (KR); Doo-Hyeon Moon, Gyeonggi-do (KR); Hee-Sook Kim, Gyeonggi-do (KR); Su-Hyun Lee, Gyeonggi-do (KR); Hyo-Nim Shin, Gyeonggi-do (KR); Kyung-Joo Lee, Seoul (KR); Kyoung-Jin Park, Gyeonggi-do (KR); Nam-Kyun Kim, Gyeonggi-do (KR); Young-Jun Cho, Gyeonggi-do (KR); Hyuck-Joo Kwon, Seoul (KR); Bong-Ok Kim, Seoul (KR)

(21) Appl. No.: 14/624,835

(22) Filed: Feb. 18, 2015

Related U.S. Application Data
(63) Continuation of application No. 14/004,089, filed on Nov. 25, 2013.

(30) Foreign Application Priority Data
Mar. 8, 2011 (KR) 1020110020492

(51) Int. Cl. H01L 51/00; C07D 403/14; C07D 409/14; C07D 405/14

(52) U.S. Cl. CPC H01L 51/0072; C07D 405/14; H01L 51/0073; C07D 403/14; C07D 409/14; H01L 51/0074; H01L 51/0052; H01L 51/5092

(57) ABSTRACT

The present invention relates to a novel organic electroluminescent compound and an organic electroluminescent device using the same. Said organic luminescent compound provides an organic electroluminescent device which has high luminous efficiency and a long operation lifetime and requires a low driving voltage improving power efficiency and power consumption.

### CROSS-REFERENCE TO RELATED APPLICATIONS

This application is a continuation of U.S. application Ser. No. 14/004,089, filed Nov. 25, 2013, which is the §371 national stage entry of PCT/KR2012/01712, filed on Mar. 8, 2012, and which claims priority to Korean Application No. 1020110020492, filed on Mar. 8, 2011. The entire contents of each of the above-identified applications are incorporated herein by reference.

### FIELD OF THE INVENTION

The present invention relates to novel organic electroluminescent compounds and organic electroluminescent device using the same.

### BACKGROUND OF THE INVENTION

An electroluminescent (EL) device is a self-light-emitting device which has advantages over other types of display devices in that it provides a wider viewing angle, a greater contrast ratio, and has a faster response time. An organic EL device was first developed by Eastman Kodak, by using small molecules (aromatic diamines) and aluminum complexes in a light-emitting layer [Appl. Phys. Lett. 51, 913, 1987].

The most important factor to determine luminous efficiency in an organic EL device is a light-emitting materials. Until now, fluorescent materials have been widely used as light-emitting material. However, in view of electroluminescent mechanisms, phosphorescent materials theoretically show four (4) times higher luminous efficiency than fluorescent materials. Thus, recently, phosphorescent materials have been investigated.

Iridium(III) complexes have been widely known as phosphorescent material, including bis(2-(2′-benzothienyl)-pyridinato-N,C3′)iridium(acetylacetonate)((acac)Ir(btp)₂), tris(2-phenylpyridine)iridium (Ir(ppy)₃) and bis(4,6-difluorophenylpyridinato-N,C2)picolinate iridium (Firpic) as red, green and blue materials, respectively.

In order to improve color purity, luminous efficiency and stability, light-emitting materials can be used as one prepared by mixing a dopant with a host material. In the host material/dopant system, the host material has a great influence on the efficiency and performance of an EL device, and thus is important.

At present, 4,4′-N,N′-dicarbazol-biphenyl (CBP) is the most widely known host material for phosphorescent materials. Further, Pioneer (Japan) developed a high performance organic EL device employing, as a host material, bathocuproine (BCP) or aluminum(III)bis(2-methyl-8-quinolinate)(4-phenylphenolate) (BAIq) which had been a material used for a hole blocking layer.

Though these phosphorous host materials provide good light-emitting characteristics, they have the following disadvantages: (1) Due to their low glass transition temperature and poor thermal stability, their degradation may occur during a high-temperature deposition process in a vacuum. (2) The power efficiency of an organic EL device is given by [(rr/voltage)×current efficiency], and thus the power efficiency is inversely proportional to the voltage. Though an organic EL device comprising phosphorescent materials provides better current efficiency (cd/A) than one comprising fluorescent materials, a significantly high driving voltage is required to be applied to an organic EL device, thereby resulting in poor power efficiency (lm/W). (3) Further, the operation lifetime of an organic EL device is short and luminous efficiency is still required to be improved.

International Patent Publication No. WO 2006/049013 discloses compounds for organic electroluminescent materials whose backbone has a condensed bicycle group. However, it does not disclose compounds having a nitrogen-containing condensed bicyclic group, which is formed by condensing two 6-membered rings; a carbazolic group; and an aryl or heteroaryl group. Further, an organic EL device comprising said compounds fails to provide good luminous efficiency, operation lifetime and driving voltage.

### DISCLOSURE OF THE INVENTION

### Technical Problem

An object of the present invention is to provide organic electroluminescent compounds imparting excellent luminous efficiency, long operation lifetime and low driving voltage to a device; and an organic electroluminescent device using said compounds.

### Solution to the Problem

The present inventors found that the above object can be achieved by a compound represented by the following formula 1:

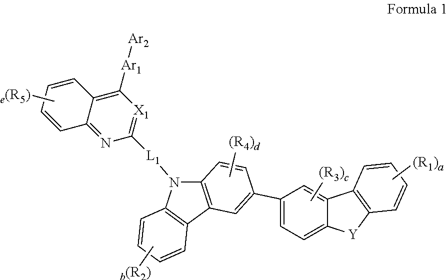

wherein

L₁ represents a single bond, a substituted or unsubstituted 5- to 30-membered heteroarylene group, a substituted or unsubstituted (C6-C30)arylene group, or a substituted or unsubstituted (C6-C30)cycloalkylene group;

X₁ represents CH or N;

Y represents —O—, —S—, —CR₁₁R₁₂— or —NR₁₃—;

Ar₁ represents a single bond, a substituted or unsubstituted 5- to 30-membered heteroarylene group, a substituted or unsubstituted (C6-C30)arylene group, or a substituted or unsubstituted (C1-C30)alkylene group;

Ar₂ represents hydrogen, deuterium, a substituted or unsubstituted (C1-C30)alkyl group, a substituted or unsubstituted (C6-C30)aryl group, or a substituted or unsubstituted 5- to 30-membered heteroaryl group;

R₁ to R₅ each independently represent hydrogen, deuterium, a halogen, a substituted or unsubstituted (C1-C30)alkyl group, a substituted or unsubstituted (C6-C30)aryl group, a substituted or unsubstituted 5- to 30-membered heteroaryl group, a substituted or unsubstituted (C3-C30)cycloalkyl group, a substituted or unsubstituted 5- to 7-membered heterocycloalkyl group, a substituted or unsubstituted (C6-C30)aryl(C1-C30)alkyl group, a substituted or unsubstituted (C6-C30)aryl group fused with at least one (C3-C30)cycloalkyl group, a 5- or 7-membered heterocycloalkyl group fused with at least one substituted or unsubstituted (C6-C30)aromatic ring, (C3-C30)cycloalkyl group fused with at least one substituted or unsubstituted (C6-C30)aromatic ring, —NR₁₄R₁₅, —SiR₁₆R₁₇R₁₈, —SR₁₉, —OR₂₀, a substituted or unsubstituted (C2-C30)alkenyl group, a substituted or unsubstituted (C2-C30)alkynyl group, a cyano group, a nitro group, or a hydroxyl group; or are linked to an adjacent substituent via a substituted or unsubstituted (C3-C30)alkylene group or a substituted or unsubstituted (C3-C30)alkenylene group to form a mono- or polycyclic alicyclic ring or a mono- or polycyclic aromatic ring whose carbon atom(s) may be substituted by at least one hetero atom selected from nitrogen, oxygen and sulfur;

R₁₁ to R₂₀ have the same meaning as one of R₁ to R₅;

a, b and e each independently represent an integer of 1 to 4; where a, b or e is an integer of 2 or more, each of R₁, each of R₂ or each of R₅ is the same or different;

c and d each independently represent an integer of 1 to 3; where c or d is an integer of 2 or more, each of R₃ or each of R₄ is the same or different; and

the heterocycloalkyl group and the heteroaryl(ene) group contain at least one hetero atom selected from B, N, O, S, P(═O), Si and P.

Herein, “(C1-C30)alkyl(ene)” is a linear or branched alkyl(ene) having 1 to 30, preferably 1 to 20, more preferable 1 to 10 carbon atoms and includes methyl, ethyl, n-propyl, isopropyl, n-butyl, isobutyl, tert-butyl, etc.; “(C2-C30) alkenyl(ene)” is a linear or branched alkenyl(ene) having 2 to 30, preferably 2 to 20, more preferably 1 to 10 carbon atoms and includes vinyl, 1-propenyl, 2-propenyl, 1-butenyl, 2-butenyl, 3-butenyl, 2-methylbut-2-enyl, etc.; “(C2-C30)alkynyl” is a linear or branched alkynyl having 2 to 30, preferably 2 to 20, more preferably 1 to 10 carbon atoms and includes ethynyl, 1-propynyl, 2-propynyl, 1-butynyl, 2-butynyl, 3-butynyl, 1-methylpent-2-ynyl, etc.; “(C1-C30)alkoxy” is a linear or branched alkoxy having 1 to 30, preferably 2 to 20, more preferably 2 to 10 carbon atoms and includes methoxy, ethoxy, propoxy, isopropoxy, 1-ethylpropoxy, etc.; “(C3-C30)cycloalkyl” is a mono- or polycyclic hydrocarbon having 3 to 30, preferably 3 to 20, more preferably 3 to 7 carbon atoms and includes cyclopropyl, cyclobutyl, cyclopentyl, cyclohexyl, etc.; “(C6-C30)cycloalkylene” is one formed by removing hydrogen from cycloalkyl having 6 to 30, preferably 6 to 20, more preferably 6 or 7 carbon atoms; and “5- to 7-membered heterocycloalkyl” is a cycloalkyl having at least one hetero atom selected from B, N, O, S, P(═O), Si and P, preferably N, O and S, and carbon atoms as remaining ring backbone atoms other than said hetero atom and includes tetrahydrofuran, pyrrolidine, tetrahydropyran, etc. Further, “(C6-C30)aryl(ene)” is a monocyclic ring or fused ring derived from an aromatic hydrocarbon and having preferably 6 to 20 ring backbone carbon atoms; and includes phenyl, biphenyl, terphenyl, naphthyl, fluorenyl, phenanthrenyl, anthracenyl, indenyl, triphenylenyl, pyrenyl, tetracenyl, perylenyl, chrysenyl, naphthacenyl, fluoranthenyl, etc. Further, “5- or 30-membered heteroaryl(ene)” is an aryl having at least one, preferably 1 to 4 hetero atom selected from the group consisting of B, N, O, S, P(═O), Si and P, and carbon atoms as remaining ring backbone atoms other than said hetero atom; is a monocyclic ring or fused ring condensed with at least benzene ring; has preferably 5 to 21 ring backbone atoms; may be partially saturated; may be one formed by linking at least one heteroaryl or aryl group to a heteroaryl group via a single bond(s); and includes a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc. and a fused ring-type heteroaryl including benzofuranyl, benzothiophenyl, isobenzofuranyl, dibenzofuranyl, dibenzothiophenyl, benzoimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, phenoxazinyl, phenanthridinyl, benzodioxolyl, etc.

Preferably, substituents of formula I are as follows:

L₁ represents preferably a single bond, a substituted or unsubstituted 5- or 30-membered heteroarylene group or a substituted or unsubstituted (C6-C30)arylene group, more preferably a single bond or a substituted or unsubstituted (C6-C30)arylene group.

X represents preferably N.

Y represents preferably —O—, —S—, —CR₁₁R₁₂— (wherein R₁₁ and R₁₂ each independently represent a substituted or unsubstituted (C1-C30)alkyl group) or —NR₁₃— (wherein R₁₃ represents a halogen, deuterium, a substituted or unsubstituted (C6-C30)aryl group, or a substituted or unsubstituted 5- or 30-membered heteroaryl group).

R₁ and R₂ each independently represent hydrogen, a substituted or unsubstituted (C6-C30)aryl group, a substituted or unsubstituted 5- or 30-membered heteroaryl group, —NR₁₄R₁₅ (wherein R₁₄ and R₁₅ each independently represent a substituted or unsubstituted (C1-C30)alkyl group or a substituted or unsubstituted (C6-C30)aryl group) or a hydroxyl group, more preferably hydrogen or a substituted or unsubstituted (C6-C30)aryl group.

R₃ to R₅ each independently represent hydrogen or a substituted or unsubstituted (C1-C30)alkyl group, more preferably hydrogen.

a to e each independently represent an integer of 1.

*—Ar₁—Ar₂ is selected from the following structures:

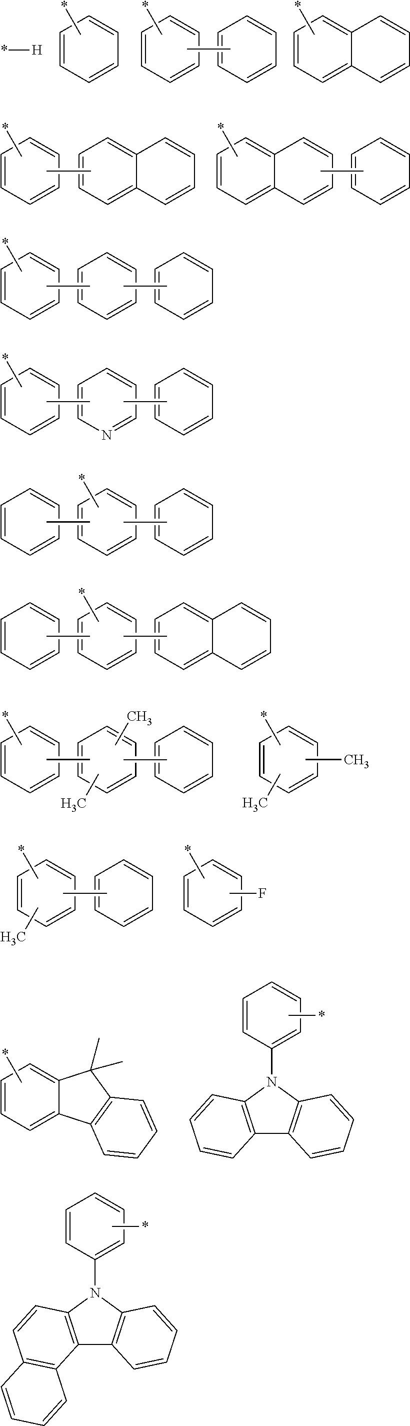

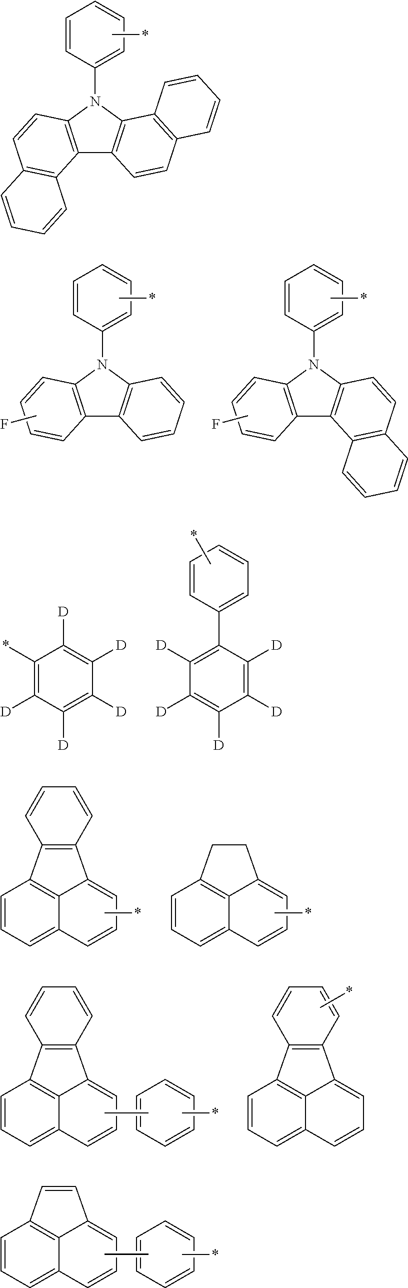

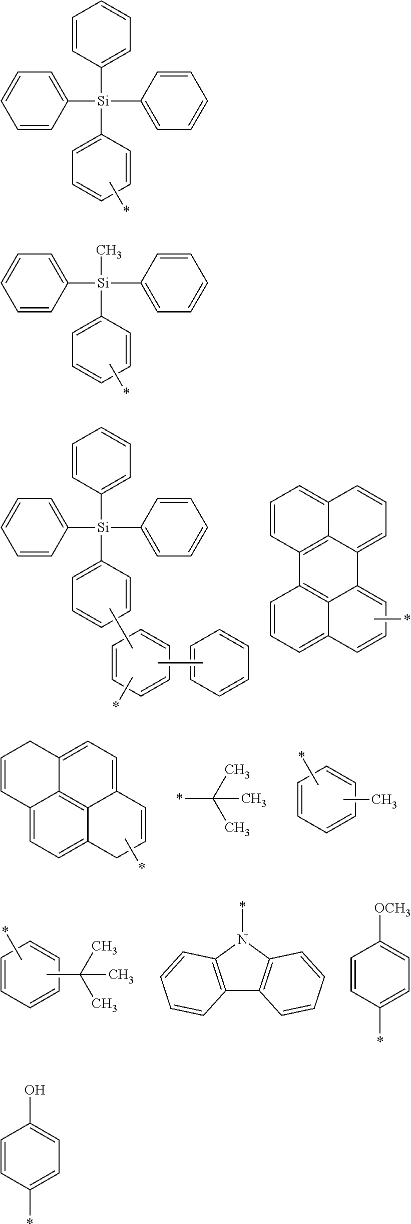

Herein, substituents of the substituted (C1-C30)alkyl group, the substituted (C2-C30)alkenyl group, the substituted (C2-C30)alkynyl group, the substituted (C6-C30)cycloalkylene group, the substituted (C3-C30)cycloalkyl group, the substituted 5- to 7-membered heterocycloalkyl group, the substituted (C6-C30)aryl(ene) group, the substituted 5- to 30-membered heteroaryl(ene) group and the substituted aromatic ring represented by said L₁, Ar₁, Ar₂, R₁ to R₅ and R₁₁ to R₂₀ each independently is at least one selected from the group consisting of deuterium, a halogen, a cyano group, a carboxyl group, a nitro group, a hydroxyl group, a (C1-C30)alkyl group, a halo(C1-C30)alkyl group, a (C2-C30)alkenyl group, a (C2-C30)alkynyl group, a (C1-C30)alkoxy group, a (C1-C30)alkylthio group, a (C3-C30)cycloalkyl group, a (C3-C30)cycloalkenyl group, a 5- to 7-membered heterocycloalkyl group, a (C6-C30)aryl group, a (C6-C30)aryloxy group, a (C6-C30)arylthio group, a 5- to 30-membered heteroaryl group, a 5- to 30-membered heteroaryl group substituted by a (C6-C30)aryl group, a (C6-C30)aryl group substituted by a 5- to 30-membered heteroaryl group, a tri(C1-C30)alkylsilyl group, a tri(C6-C30)arylsilyl group, a di(C1-C30)alkyl(C6-C30)arylsilyl group, a (C1-C30)alkyldi(C6-C30)arylsilyl group, an amino group, a mono or di(C1-C30)alkylamino group, a mono or di(C6-C30)arylamino group, a (C1-C30)alkyl(C6-C30)arylamino group, a (C1-C30)alkylcarbonyl group, a (C1-C30)alkoxycarbonyl group, a (C1-C30)arylcarbonyl group, a di(C6-C30)arylbornyl group, a di(C1-C30)alkylbornyl group, a (C1-C30)alkyl(C6-C30)arylbornyl group, a (C6-C30)aryl(C1-C30)alkyl group and a (C1-C30)alkyl(C6-C30)aryl group. Preferably, said substituents are at least one selected from the group consisting of deuterium, a halogen, a (C1-C30)alkyl group, a halo(C1-C30)alkyl group, a (C6-C30)aryl group, a 5- to 30-membered heteroaryl group, a tri(C1-C30)alkylsilyl group, a tri(C6-C30)arylsilyl group, a di(C1-C30)alkyl(C6-C30)arylsilyl group, a (C1-C30)alkyldi(C6-C30)arylsilyl group, a hydroxyl group and a (C1-C30)alkoxy group.

Organic electroluminescent compounds according to the present invention include the following, but are not limited thereto:

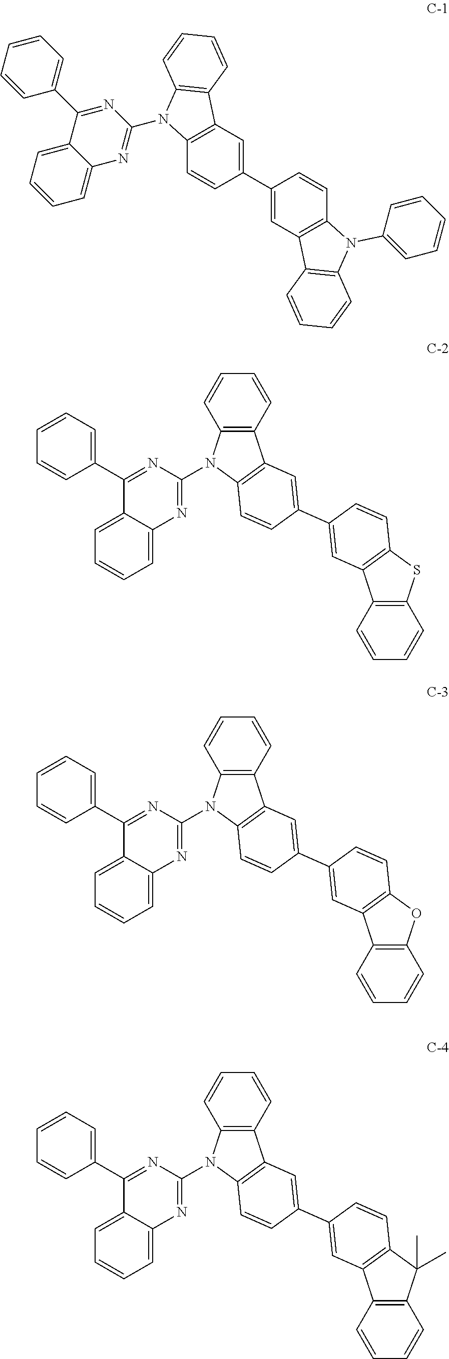 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-c2nc(-n3c4ccccc4c4cc(-c5ccc6sc7ccccc7c6c5)ccc43)nc3ccccc23)cc1 ; c1ccc(-c2nc(-n3c4ccccc4c4cc(-c5ccc6oc7ccccc7c6c5)ccc43)nc3ccccc23)cc1 ; CC1(C)c2ccccc2-c2cc(-c3ccc4c(c3)c3ccccc3n4-c3nc(-c4ccccc4)c4ccccc4n3)ccc21]

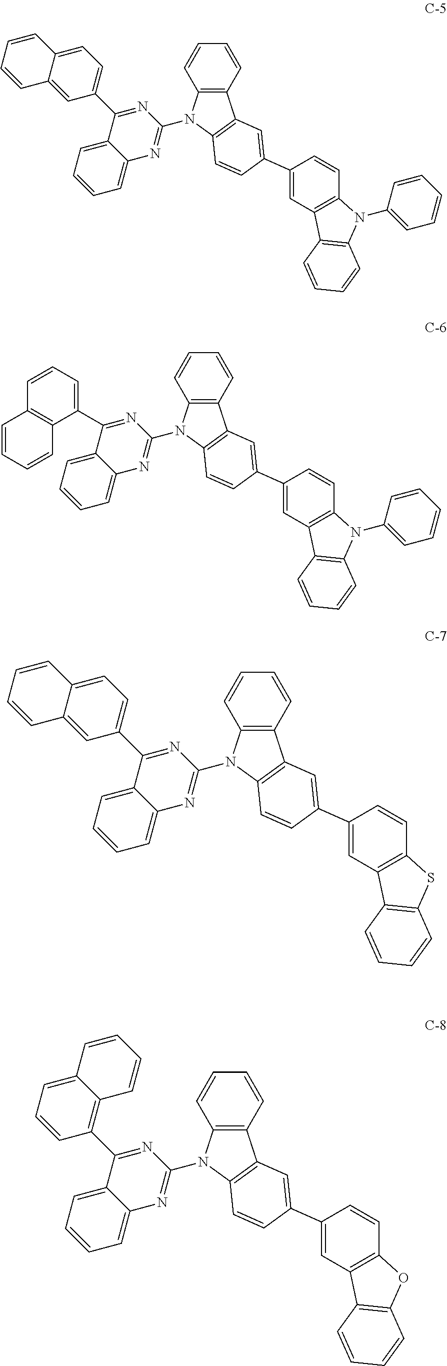 [SMILES: c1ccc(-n2c3ccccc3c3cc(-c4ccc5c(c4)c4ccccc4n5-c4nc(-c5ccc6ccccc6c5)c5ccccc5n4)ccc32)cc1 ; c1ccc(-n2c3ccccc3c3cc(-c4ccc5c(c4)c4ccccc4n5-c4nc(-c5cccc6ccccc56)c5ccccc5n4)ccc32)cc1 ; c1ccc2cc(-c3nc(-n4c5ccccc5c5cc(-c6ccc7sc8ccccc8c7c6)ccc54)nc4ccccc34)ccc2c1 ; c1ccc2c(-c3nc(-n4c5ccccc5c5cc(-c6ccc7oc8ccccc8c7c6)ccc54)nc4ccccc34)cccc2c1]

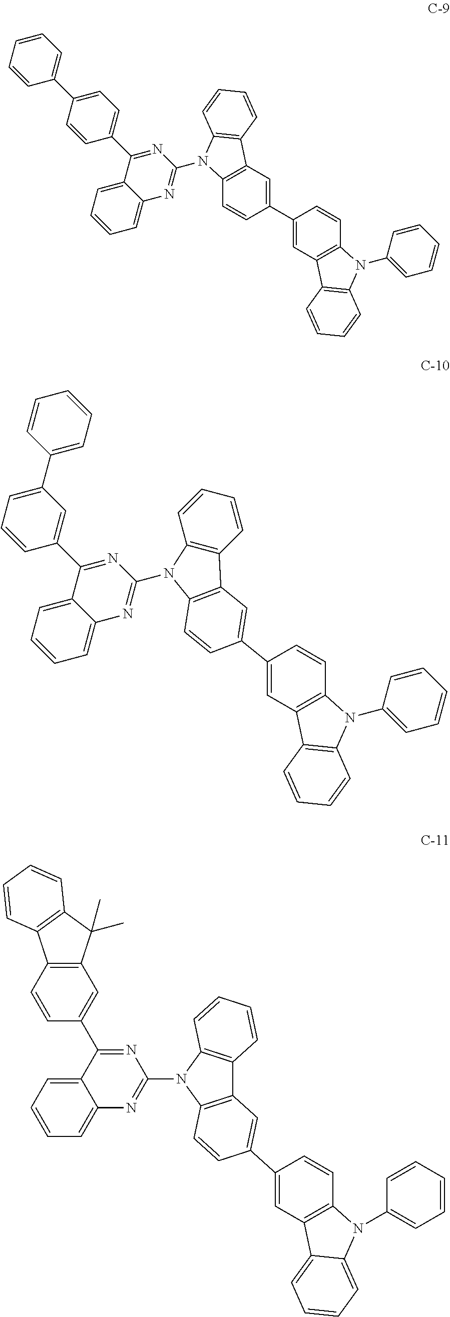 [SMILES: c1ccc(-c2ccc(-c3nc(-n4c5ccccc5c5cc(-c6ccc7c(c6)c6ccccc6n7-c6ccccc6)ccc54)nc4ccccc34)cc2)cc1 ; c1ccc(-c2cccc(-c3nc(-n4c5ccccc5c5cc(-c6ccc7c(c6)c6ccccc6n7-c6ccccc6)ccc54)nc4ccccc34)c2)cc1 ; CC1(C)c2ccccc2-c2ccc(-c3nc(-n4c5ccccc5c5cc(-c6ccc7c(c6)c6ccccc6n7-c6ccccc6)ccc54)nc4ccccc34)cc21]

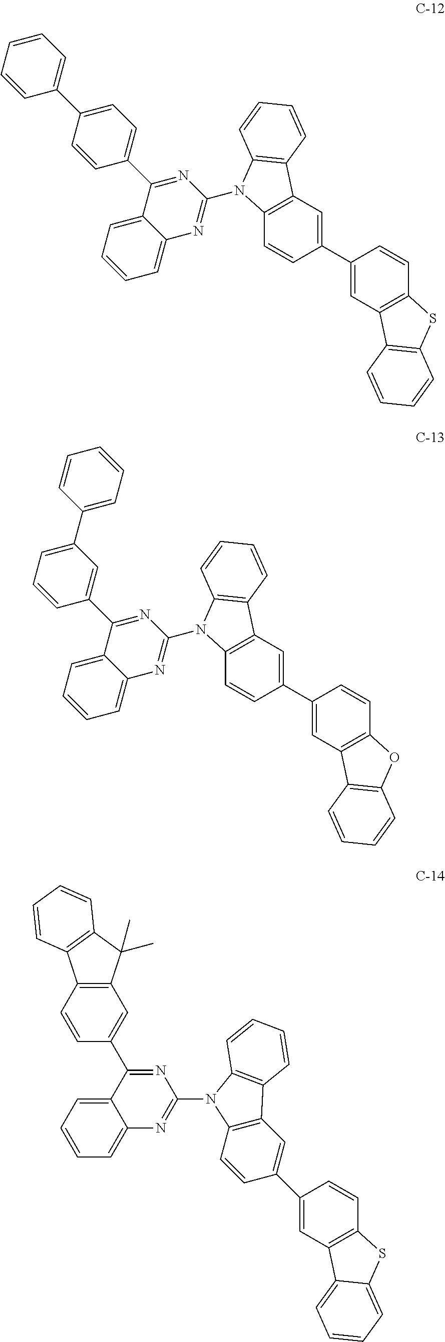 [SMILES: c1ccc(-c2ccc(-c3nc(-n4c5ccccc5c5cc(-c6ccc7sc8ccccc8c7c6)ccc54)nc4ccccc34)cc2)cc1 ; c1ccc(-c2cccc(-c3nc(-n4c5ccccc5c5cc(-c6ccc7oc8ccccc8c7c6)ccc54)nc4ccccc34)c2)cc1 ; CC1(C)c2ccccc2-c2ccc(-c3nc(-n4c5ccccc5c5cc(-c6ccc7sc8ccccc8c7c6)ccc54)nc4ccccc34)cc21]

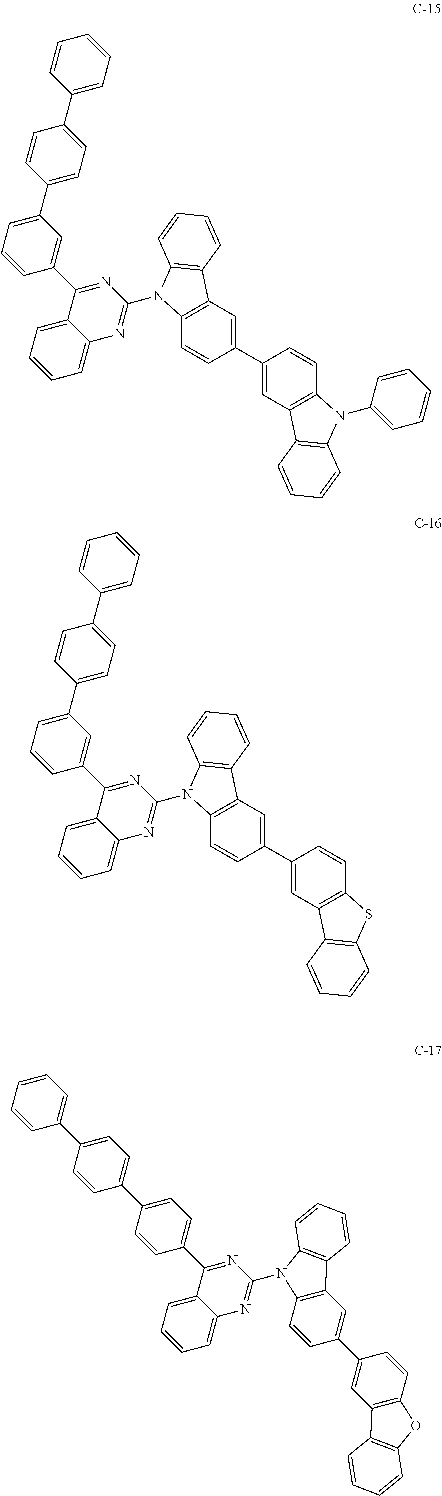 [SMILES: c1ccc(-c2ccc(-c3cccc(-c4nc(-n5c6ccccc6c6cc(-c7ccc8c(c7)c7ccccc7n8-c7ccccc7)ccc65)nc5ccccc45)c3)cc2)cc1 ; c1ccc(-c2ccc(-c3cccc(-c4nc(-n5c6ccccc6c6cc(-c7ccc8sc9ccccc9c8c7)ccc65)nc5ccccc45)c3)cc2)cc1 ; c1ccc(-c2ccc(-c3ccc(-c4nc(-n5c6ccccc6c6cc(-c7ccc8oc9ccccc9c8c7)ccc65)nc5ccccc45)cc3)cc2)cc1]

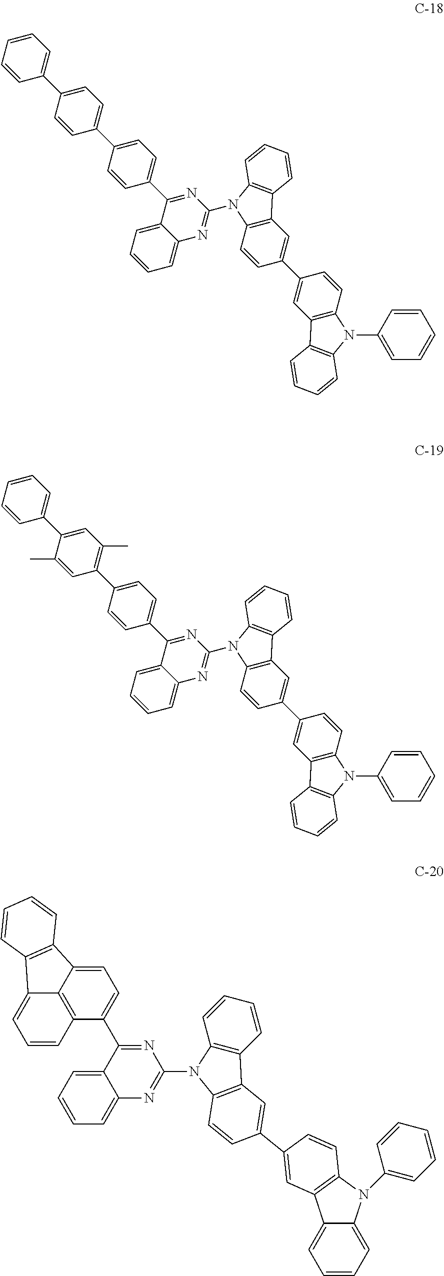 [SMILES: c1ccc(-c2ccc(-c3ccc(-c4nc(-n5c6ccccc6c6cc(-c7ccc8c(c7)c7ccccc7n8-c7ccccc7)ccc65)nc5ccccc45)cc3)cc2)cc1 ; Cc1cc(-c2ccc(-c3nc(-n4c5ccccc5c5cc(-c6ccc7c(c6)c6ccccc6n7-c6ccccc6)ccc54)nc4ccccc34)cc2)c(C)cc1-c1ccccc1 ; c1ccc(-n2c3ccccc3c3cc(-c4ccc5c(c4)c4ccccc4n5-c4nc(-c5ccc6c7c(cccc57)-c5ccccc5-6)c5ccccc5n4)ccc32)cc1]

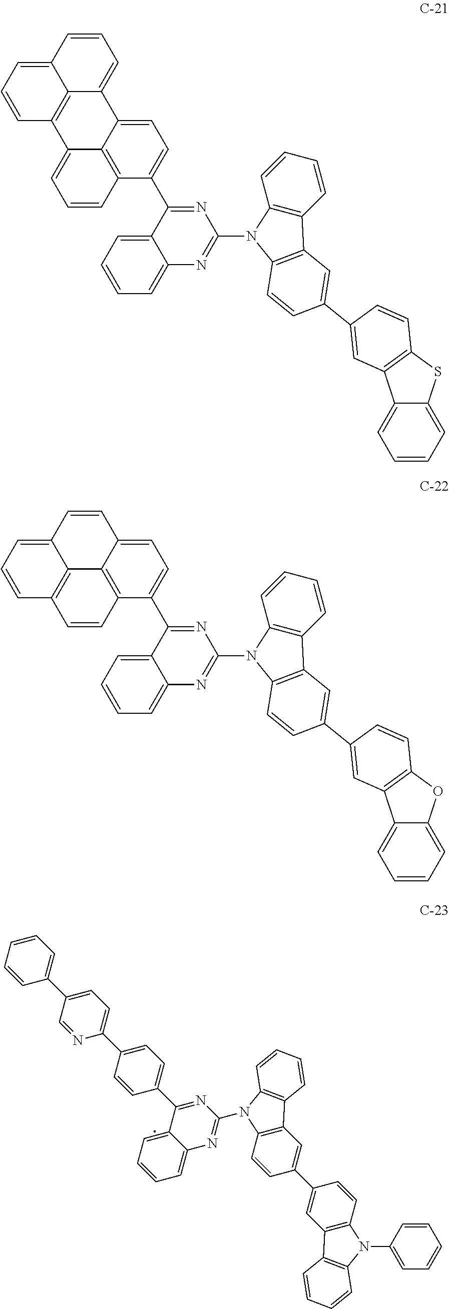

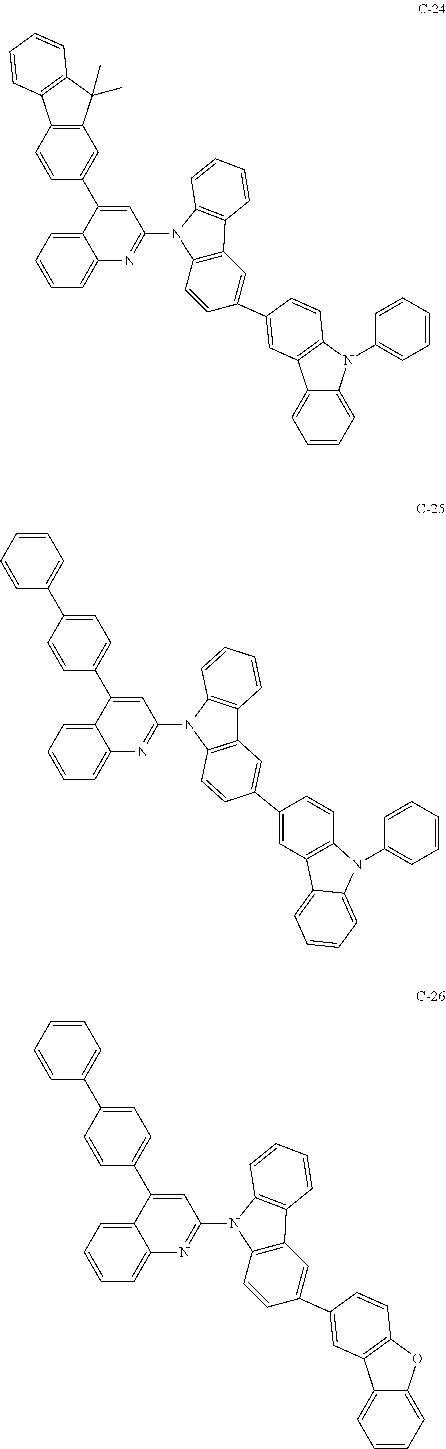 [SMILES: CC1(C)c2ccccc2-c2ccc(-c3cc(-n4c5ccccc5c5cc(-c6ccc7c(c6)c6ccccc6n7-c6ccccc6)ccc54)nc4ccccc34)cc21 ; c1ccc(-c2ccc(-c3cc(-n4c5ccccc5c5cc(-c6ccc7c(c6)c6ccccc6n7-c6ccccc6)ccc54)nc4ccccc34)cc2)cc1 ; c1ccc(-c2ccc(-c3cc(-n4c5ccccc5c5cc(-c6ccc7oc8ccccc8c7c6)ccc54)nc4ccccc34)cc2)cc1]

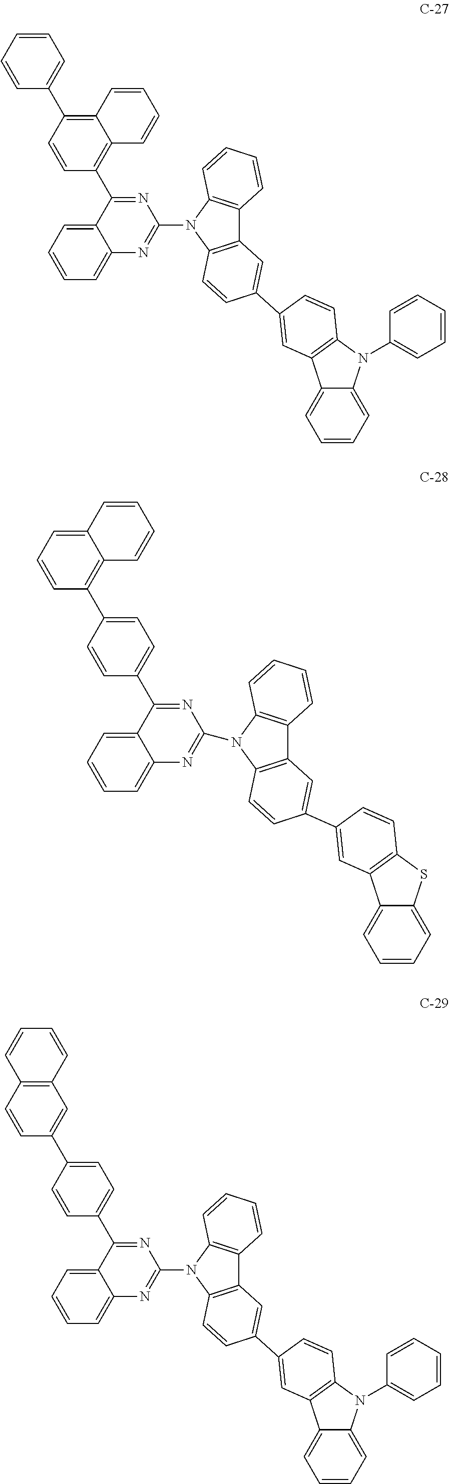 [SMILES: c1ccc(-c2ccc(-c3nc(-n4c5ccccc5c5cc(-c6ccc7c(c6)c6ccccc6n7-c6ccccc6)ccc54)nc4ccccc34)c3ccccc23)cc1 ; c1ccc2c(-c3ccc(-c4nc(-n5c6ccccc6c6cc(-c7ccc8sc9ccccc9c8c7)ccc65)nc5ccccc45)cc3)cccc2c1 ; c1ccc(-n2c3ccccc3c3cc(-c4ccc5c(c4)c4ccccc4n5-c4nc(-c5ccc(-c6ccc7ccccc7c6)cc5)c5ccccc5n4)ccc32)cc1]

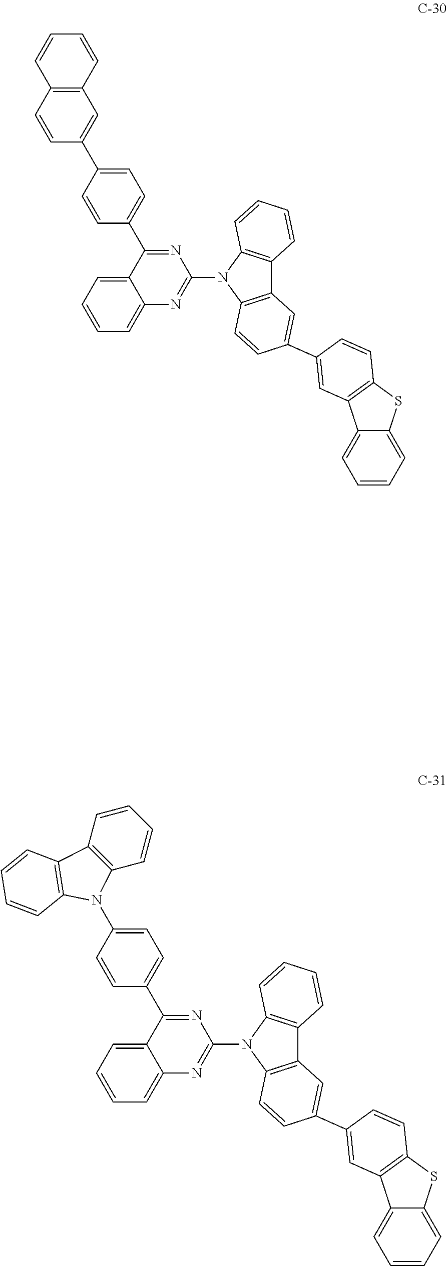 [SMILES: c1ccc2cc(-c3ccc(-c4nc(-n5c6ccccc6c6cc(-c7ccc8sc9ccccc9c8c7)ccc65)nc5ccccc45)cc3)ccc2c1 ; c1ccc2c(-c3ccc(-n4c5ccccc5c5ccccc54)cc3)nc(-n3c4ccccc4c4cc(-c5ccc6sc7ccccc7c6c5)ccc43)nc2c1]

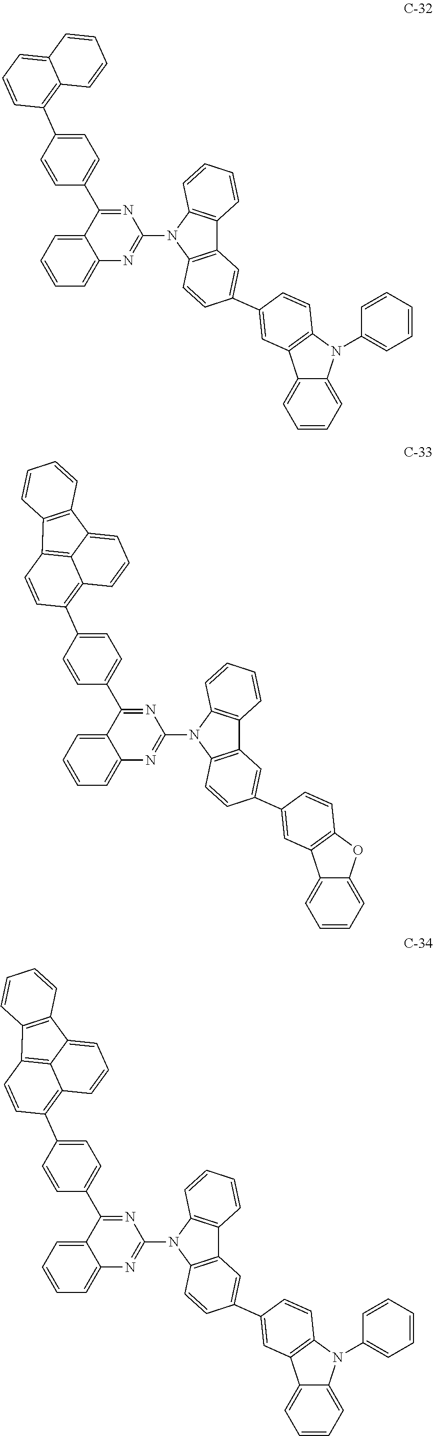 [SMILES: c1ccc(-n2c3ccccc3c3cc(-c4ccc5c(c4)c4ccccc4n5-c4nc(-c5ccc(-c6cccc7ccccc67)cc5)c5ccccc5n4)ccc32)cc1 ; c1ccc2c(c1)-c1cccc3c(-c4ccc(-c5nc(-n6c7ccccc7c7cc(-c8ccc9oc%10ccccc%10c9c8)ccc76)nc6ccccc56)cc4)ccc-2c13 ; c1ccc(-n2c3ccccc3c3cc(-c4ccc5c(c4)c4ccccc4n5-c4nc(-c5ccc(-c6ccc7c8c(cccc68)-c6ccccc6-7)cc5)c5ccccc5n4)ccc32)cc1]

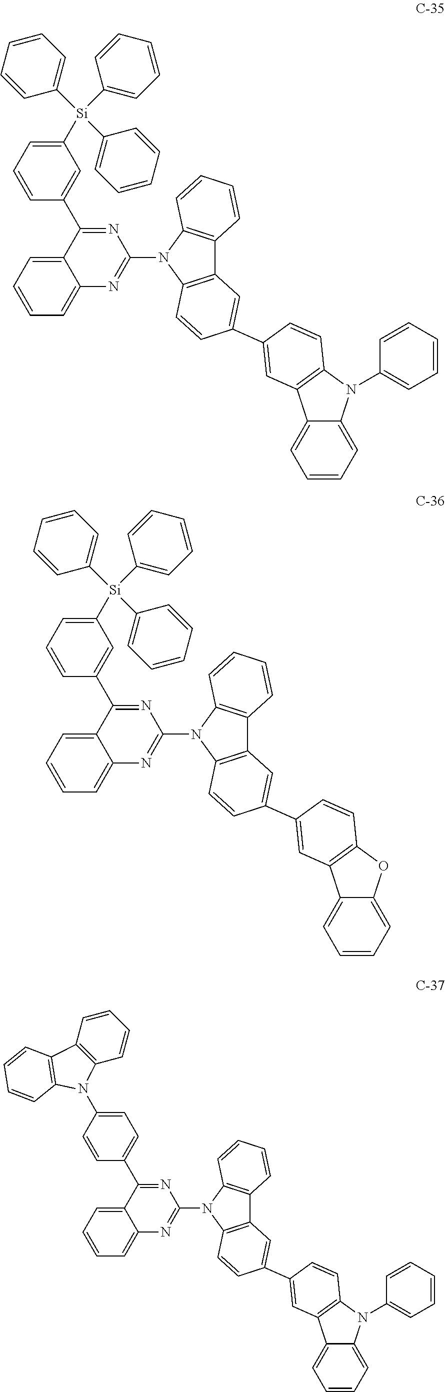 [SMILES: c1ccc(-n2c3ccccc3c3cc(-c4ccc5c(c4)c4ccccc4n5-c4nc(-c5cccc([Si](c6ccccc6)(c6ccccc6)c6ccccc6)c5)c5ccccc5n4)ccc32)cc1 ; c1ccc([Si](c2ccccc2)(c2ccccc2)c2cccc(-c3nc(-n4c5ccccc5c5cc(-c6ccc7oc8ccccc8c7c6)ccc54)nc4ccccc34)c2)cc1 ; c1ccc(-n2c3ccccc3c3cc(-c4ccc5c(c4)c4ccccc4n5-c4nc(-c5ccc(-n6c7ccccc7c7ccccc76)cc5)c5ccccc5n4)ccc32)cc1]

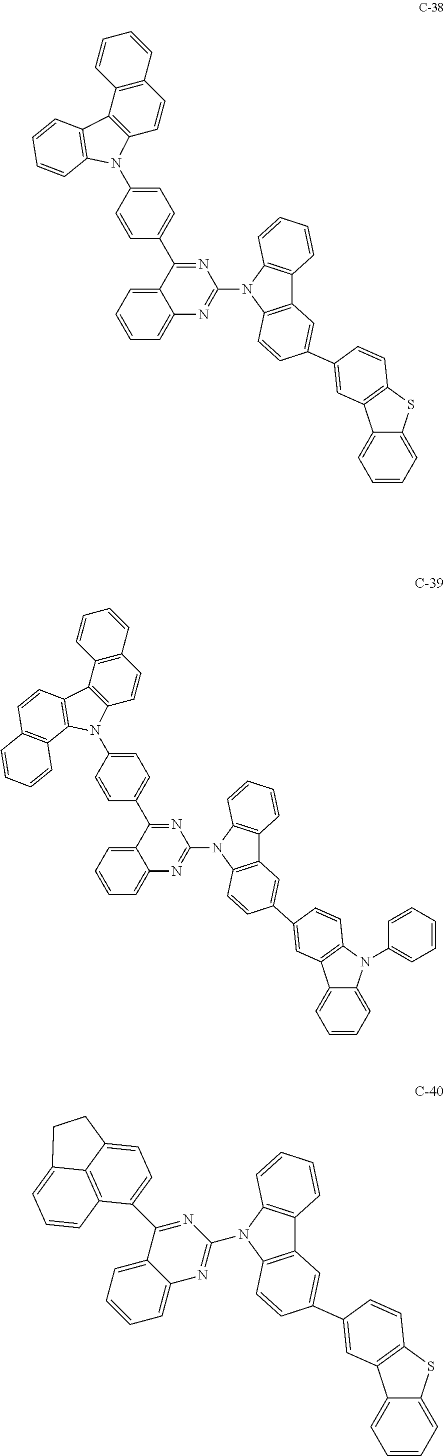 [SMILES: c1ccc2c(c1)ccc1c2c2ccccc2n1-c1ccc(-c2nc(-n3c4ccccc4c4cc(-c5ccc6sc7ccccc7c6c5)ccc43)nc3ccccc23)cc1 ; c1ccc(-n2c3ccccc3c3cc(-c4ccc5c(c4)c4ccccc4n5-c4nc(-c5ccc(-n6c7ccc8ccccc8c7c7ccc8ccccc8c76)cc5)c5ccccc5n4)ccc32)cc1 ; c1ccc2c(-c3ccc4c5c(cccc35)CC4)nc(-n3c4ccccc4c4cc(-c5ccc6sc7ccccc7c6c5)ccc43)nc2c1]

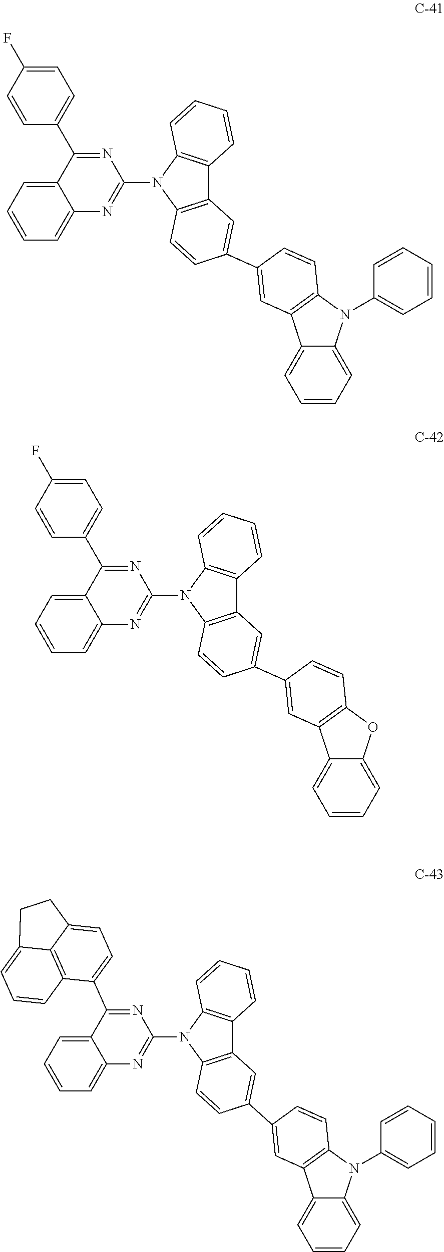 [SMILES: Fc1ccc(-c2nc(-n3c4ccccc4c4cc(-c5ccc6c(c5)c5ccccc5n6-c5ccccc5)ccc43)nc3ccccc23)cc1 ; Fc1ccc(-c2nc(-n3c4ccccc4c4cc(-c5ccc6oc7ccccc7c6c5)ccc43)nc3ccccc23)cc1 ; c1ccc(-n2c3ccccc3c3cc(-c4ccc5c(c4)c4ccccc4n5-c4nc(-c5ccc6c7c(cccc57)CC6)c5ccccc5n4)ccc32)cc1]

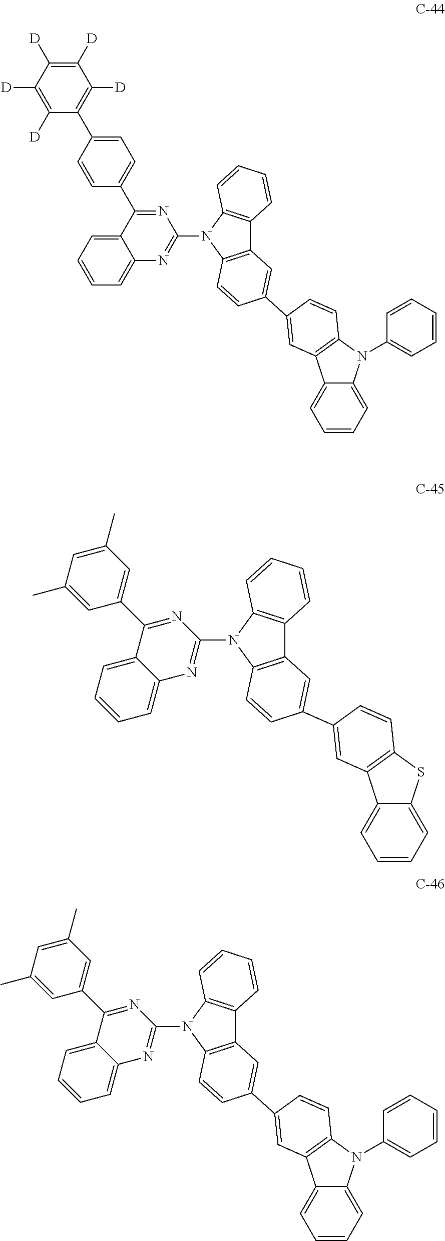 [SMILES: [2H]c1c([2H])c([2H])c(-c2ccc(-c3nc(-n4c5ccccc5c5cc(-c6ccc7c(c6)c6ccccc6n7-c6ccccc6)ccc54)nc4ccccc34)cc2)c([2H])c1[2H] ; Cc1cc(C)cc(-c2nc(-n3c4ccccc4c4cc(-c5ccc6sc7ccccc7c6c5)ccc43)nc3ccccc23)c1 ; Cc1cc(C)cc(-c2nc(-n3c4ccccc4c4cc(-c5ccc6c(c5)c5ccccc5n6-c5ccccc5)ccc43)nc3ccccc23)c1]

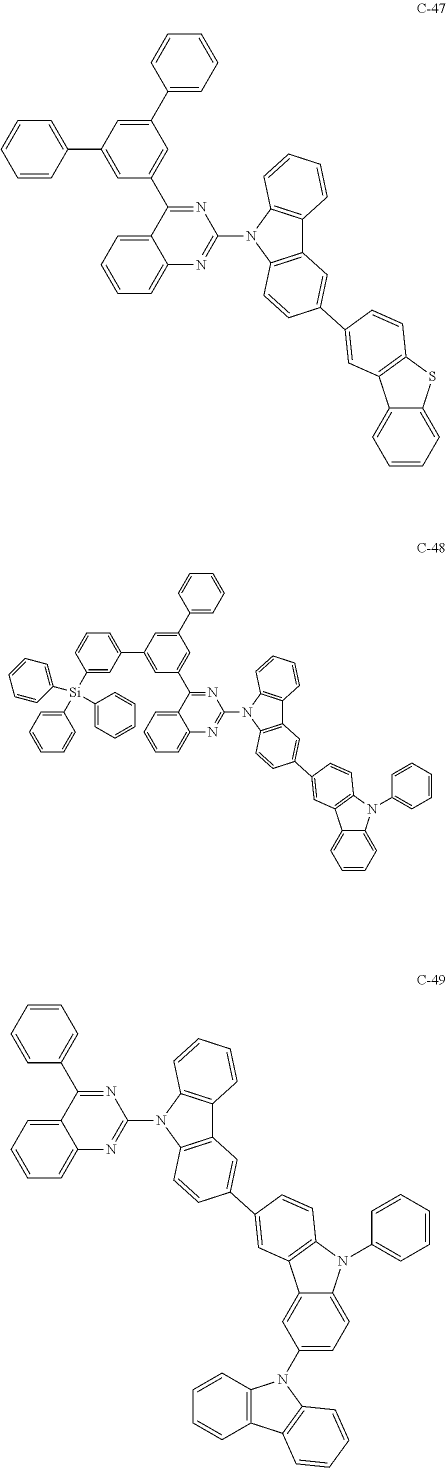 [SMILES: c1ccc(-c2cc(-c3ccccc3)cc(-c3nc(-n4c5ccccc5c5cc(-c6ccc7sc8ccccc8c7c6)ccc54)nc4ccccc34)c2)cc1 ; c1ccc(-c2cc(-c3cccc([Si](c4ccccc4)(c4ccccc4)c4ccccc4)c3)cc(-c3nc(-n4c5ccccc5c5cc(-c6ccc7c(c6)c6ccccc6n7-c6ccccc6)ccc54)nc4ccccc34)c2)cc1 ; c1ccc(-c2nc(-n3c4ccccc4c4cc(-c5ccc6c(c5)c5cc(-n7c8ccccc8c8ccccc87)ccc5n6-c5ccccc5)ccc43)nc3ccccc23)cc1]

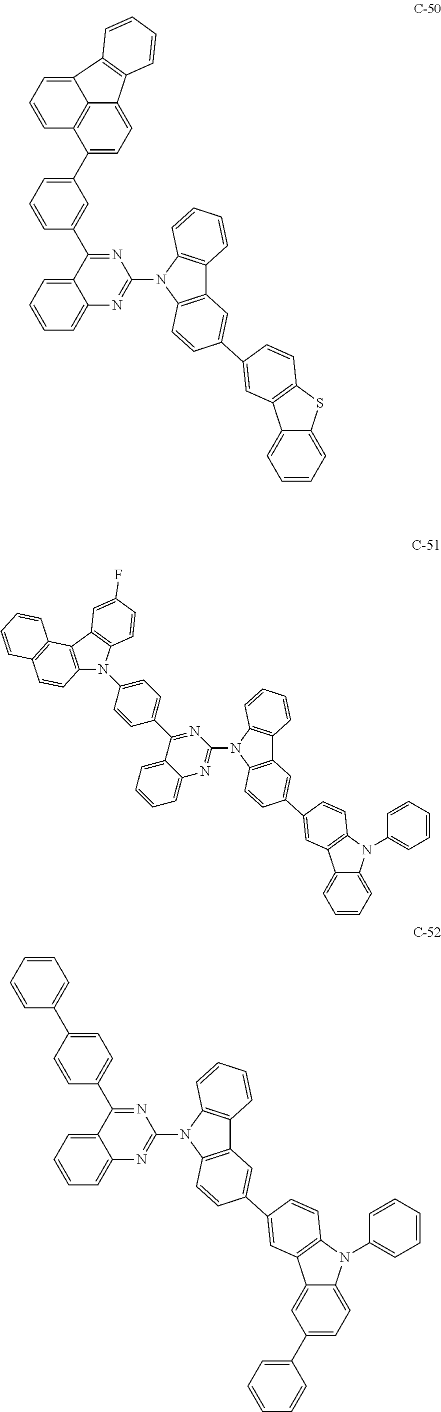 [SMILES: c1cc(-c2nc(-n3c4ccccc4c4cc(-c5ccc6sc7ccccc7c6c5)ccc43)nc3ccccc23)cc(-c2ccc3c4c(cccc24)-c2ccccc2-3)c1 ; Fc1ccc2c(c1)c1c3ccccc3ccc1n2-c1ccc(-c2nc(-n3c4ccccc4c4cc(-c5ccc6c(c5)c5ccccc5n6-c5ccccc5)ccc43)nc3ccccc23)cc1 ; c1ccc(-c2ccc(-c3nc(-n4c5ccccc5c5cc(-c6ccc7c(c6)c6cc(-c8ccccc8)ccc6n7-c6ccccc6)ccc54)nc4ccccc34)cc2)cc1]

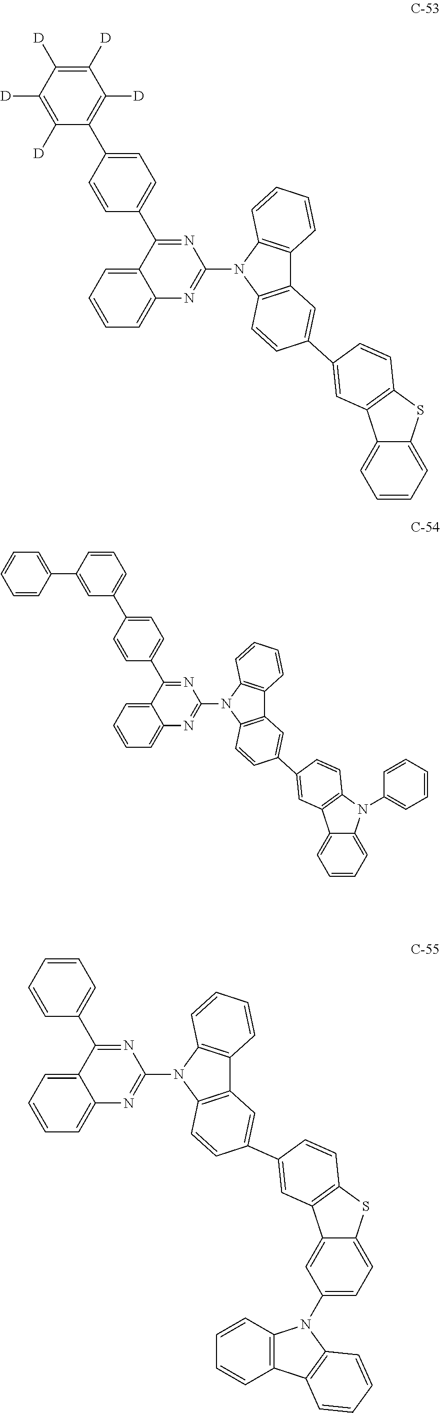 [SMILES: [2H]c1c([2H])c([2H])c(-c2ccc(-c3nc(-n4c5ccccc5c5cc(-c6ccc7sc8ccccc8c7c6)ccc54)nc4ccccc34)cc2)c([2H])c1[2H] ; c1ccc(-c2cccc(-c3ccc(-c4nc(-n5c6ccccc6c6cc(-c7ccc8c(c7)c7ccccc7n8-c7ccccc7)ccc65)nc5ccccc45)cc3)c2)cc1 ; c1ccc(-c2nc(-n3c4ccccc4c4cc(-c5ccc6sc7ccc(-n8c9ccccc9c9ccccc98)cc7c6c5)ccc43)nc3ccccc23)cc1]

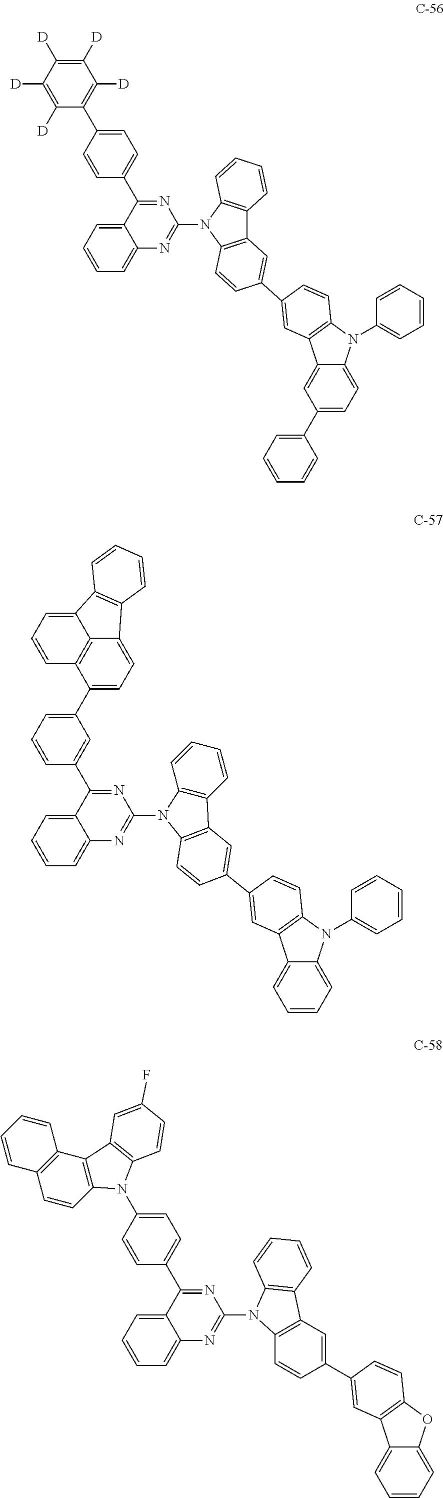 [SMILES: [2H]c1c([2H])c([2H])c(-c2ccc(-c3nc(-n4c5ccccc5c5cc(-c6ccc7c(c6)c6cc(-c8ccccc8)ccc6n7-c6ccccc6)ccc54)nc4ccccc34)cc2)c([2H])c1[2H] ; c1ccc(-n2c3ccccc3c3cc(-c4ccc5c(c4)c4ccccc4n5-c4nc(-c5cccc(-c6ccc7c8c(cccc68)-c6ccccc6-7)c5)c5ccccc5n4)ccc32)cc1 ; Fc1ccc2c(c1)c1c3ccccc3ccc1n2-c1ccc(-c2nc(-n3c4ccccc4c4cc(-c5ccc6oc7ccccc7c6c5)ccc43)nc3ccccc23)cc1]

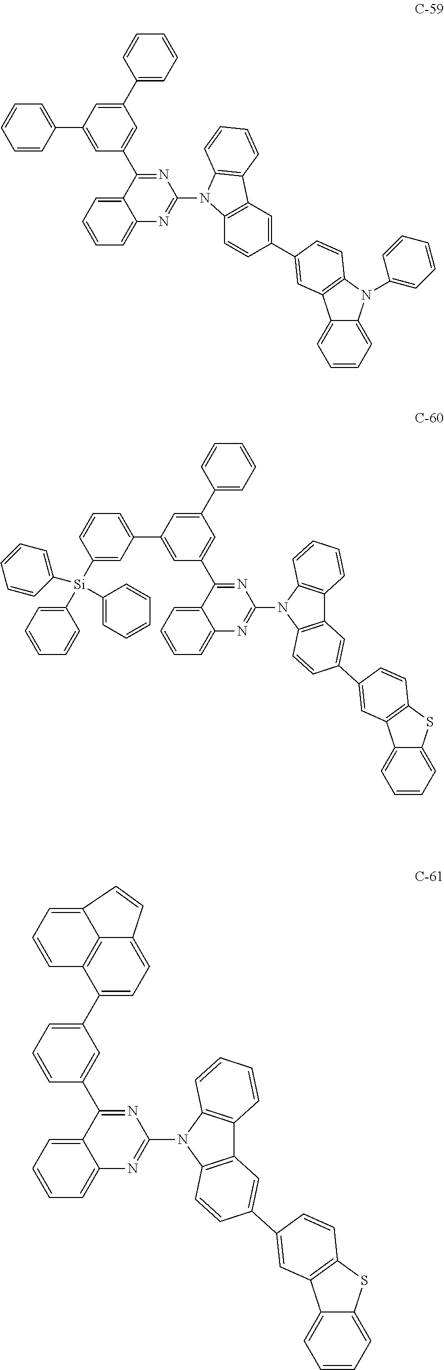 [SMILES: c1ccc(-c2cc(-c3ccccc3)cc(-c3nc(-n4c5ccccc5c5cc(-c6ccc7c(c6)c6ccccc6n7-c6ccccc6)ccc54)nc4ccccc34)c2)cc1 ; c1ccc(-c2cc(-c3cccc([Si](c4ccccc4)(c4ccccc4)c4ccccc4)c3)cc(-c3nc(-n4c5ccccc5c5cc(-c6ccc7sc8ccccc8c7c6)ccc54)nc4ccccc34)c2)cc1 ; C1=Cc2ccc(-c3cccc(-c4nc(-n5c6ccccc6c6cc(-c7ccc8sc9ccccc9c8c7)ccc65)nc5ccccc45)c3)c3cccc1c23]

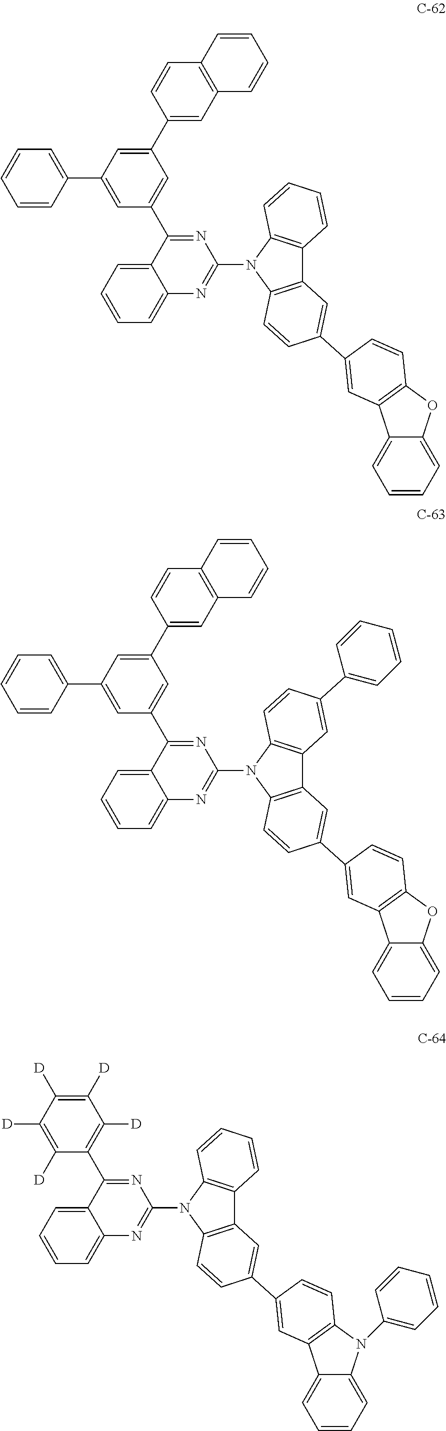 [SMILES: c1ccc(-c2cc(-c3ccc4ccccc4c3)cc(-c3nc(-n4c5ccccc5c5cc(-c6ccc7oc8ccccc8c7c6)ccc54)nc4ccccc34)c2)cc1 ; c1ccc(-c2cc(-c3ccc4ccccc4c3)cc(-c3nc(-n4c5ccc(-c6ccccc6)cc5c5cc(-c6ccc7oc8ccccc8c7c6)ccc54)nc4ccccc34)c2)cc1 ; [2H]c1c([2H])c([2H])c(-c2nc(-n3c4ccccc4c4cc(-c5ccc6c(c5)c5ccccc5n6-c5ccccc5)ccc43)nc3ccccc23)c([2H])c1[2H]]

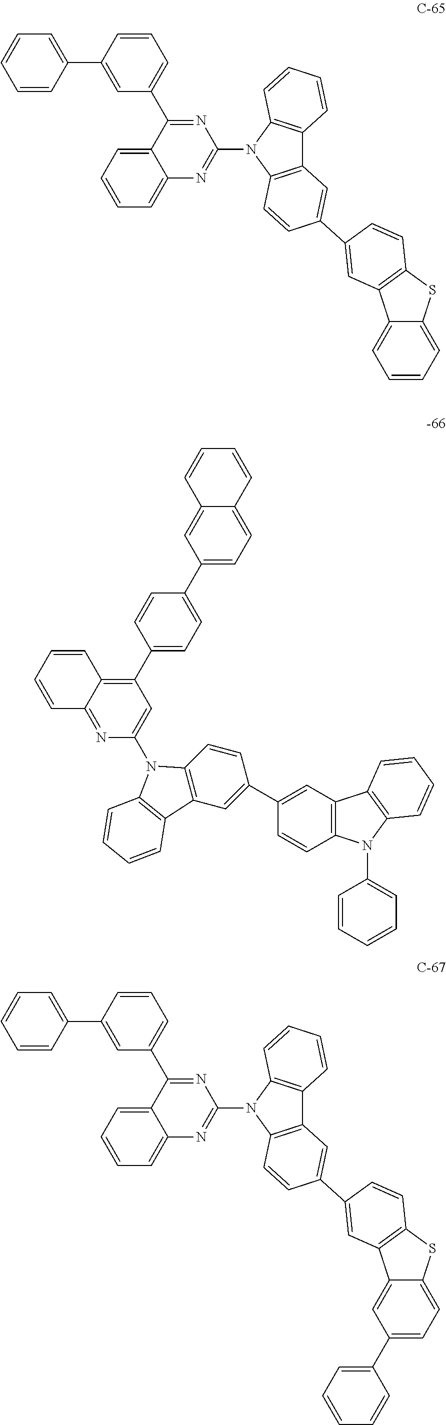 [SMILES: c1ccc(-c2cccc(-c3nc(-n4c5ccccc5c5cc(-c6ccc7sc8ccccc8c7c6)ccc54)nc4ccccc34)c2)cc1 ; c1ccc(-n2c3ccccc3c3cc(-c4ccc5c(c4)c4ccccc4n5-c4cc(-c5ccc(-c6ccc7ccccc7c6)cc5)c5ccccc5n4)ccc32)cc1 ; c1ccc(-c2cccc(-c3nc(-n4c5ccccc5c5cc(-c6ccc7sc8ccc(-c9ccccc9)cc8c7c6)ccc54)nc4ccccc34)c2)cc1]

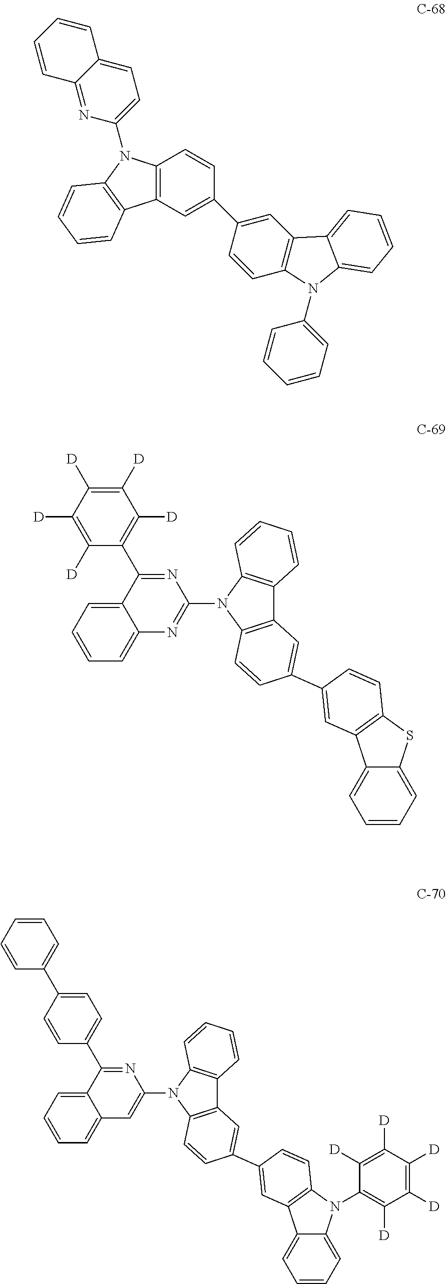 [SMILES: c1ccc(-n2c3ccccc3c3cc(-c4ccc5c(c4)c4ccccc4n5-c4ccc5ccccc5n4)ccc32)cc1 ; [2H]c1c([2H])c([2H])c(-c2nc(-n3c4ccccc4c4cc(-c5ccc6sc7ccccc7c6c5)ccc43)nc3ccccc23)c([2H])c1[2H] ; [2H]c1c([2H])c([2H])c(-n2c3ccccc3c3cc(-c4ccc5c(c4)c4ccccc4n5-c4cc5ccccc5c(-c5ccc(-c6ccccc6)cc5)n4)ccc32)c([2H])c1[2H]]

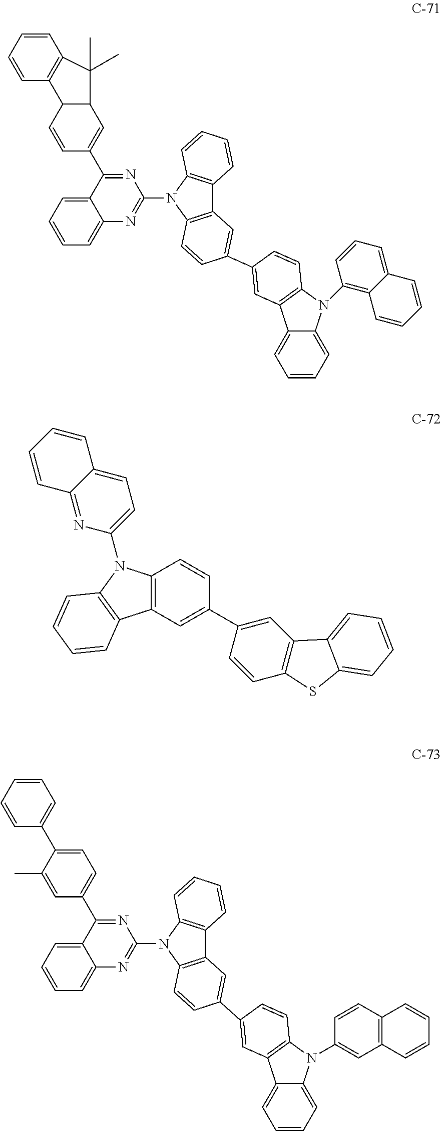 [SMILES: CC1(C)c2ccccc2C2C=CC(c3nc(-n4c5ccccc5c5cc(-c6ccc7c(c6)c6ccccc6n7-c6cccc7ccccc67)ccc54)nc4ccccc34)=CC21 ; c1ccc2nc(-n3c4ccccc4c4cc(-c5ccc6sc7ccccc7c6c5)ccc43)ccc2c1 ; Cc1cc(-c2nc(-n3c4ccccc4c4cc(-c5ccc6c(c5)c5ccccc5n6-c5ccc6ccccc6c5)ccc43)nc3ccccc23)ccc1-c1ccccc1]

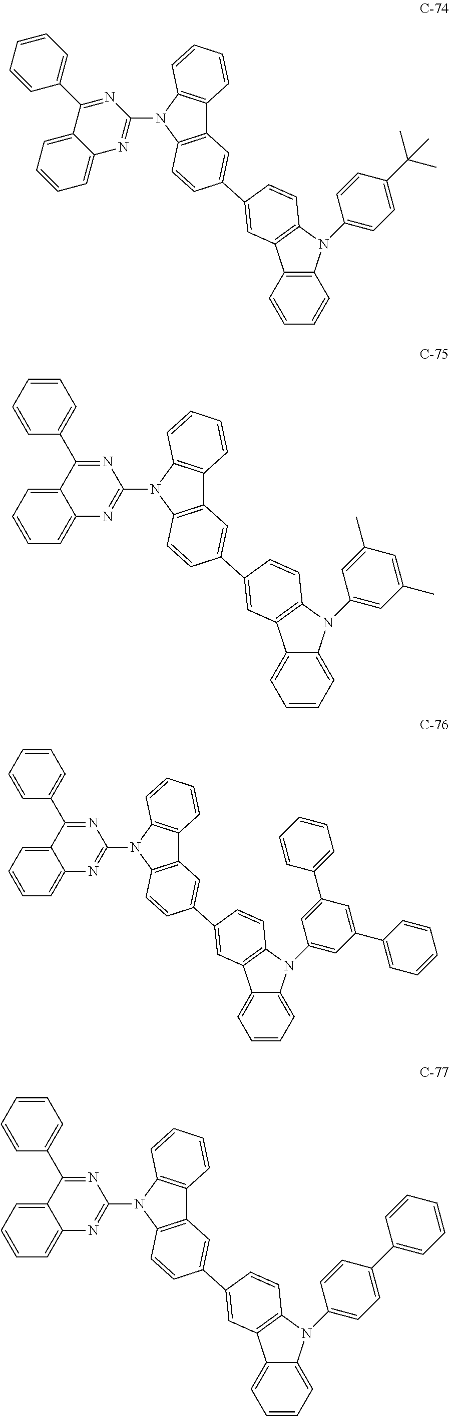 [SMILES: CC(C)(C)c1ccc(-n2c3ccccc3c3cc(-c4ccc5c(c4)c4ccccc4n5-c4nc(-c5ccccc5)c5ccccc5n4)ccc32)cc1 ; Cc1cc(C)cc(-n2c3ccccc3c3cc(-c4ccc5c(c4)c4ccccc4n5-c4nc(-c5ccccc5)c5ccccc5n4)ccc32)c1 ; c1ccc(-c2cc(-c3ccccc3)cc(-n3c4ccccc4c4cc(-c5ccc6c(c5)c5ccccc5n6-c5nc(-c6ccccc6)c6ccccc6n5)ccc43)c2)cc1 ; c1ccc(-c2ccc(-n3c4ccccc4c4cc(-c5ccc6c(c5)c5ccccc5n6-c5nc(-c6ccccc6)c6ccccc6n5)ccc43)cc2)cc1]

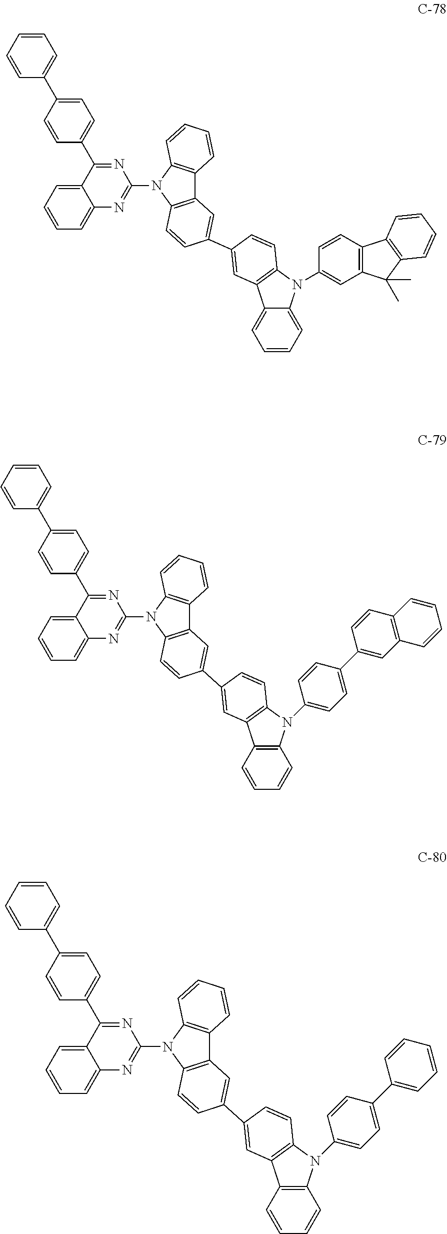 [SMILES: CC1(C)c2ccccc2-c2ccc(-n3c4ccccc4c4cc(-c5ccc6c(c5)c5ccccc5n6-c5nc(-c6ccc(-c7ccccc7)cc6)c6ccccc6n5)ccc43)cc21 ; c1ccc(-c2ccc(-c3nc(-n4c5ccccc5c5cc(-c6ccc7c(c6)c6ccccc6n7-c6ccc(-c7ccc8ccccc8c7)cc6)ccc54)nc4ccccc34)cc2)cc1 ; c1ccc(-c2ccc(-c3nc(-n4c5ccccc5c5cc(-c6ccc7c(c6)c6ccccc6n7-c6ccc(-c7ccccc7)cc6)ccc54)nc4ccccc34)cc2)cc1]

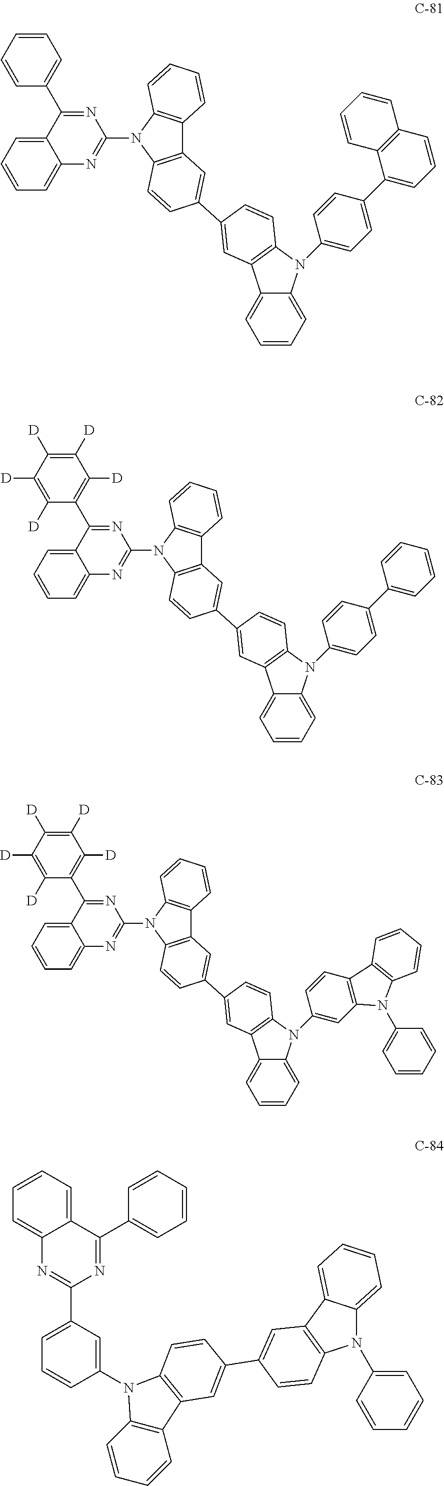 [SMILES: c1ccc(-c2nc(-n3c4ccccc4c4cc(-c5ccc6c(c5)c5ccccc5n6-c5ccc(-c6cccc7ccccc67)cc5)ccc43)nc3ccccc23)cc1 ; [2H]c1c([2H])c([2H])c(-c2nc(-n3c4ccccc4c4cc(-c5ccc6c(c5)c5ccccc5n6-c5ccc(-c6ccccc6)cc5)ccc43)nc3ccccc23)c([2H])c1[2H] ; [2H]c1c([2H])c([2H])c(-c2nc(-n3c4ccccc4c4cc(-c5ccc6c(c5)c5ccccc5n6-c5ccc6c7ccccc7n(-c7ccccc7)c6c5)ccc43)nc3ccccc23)c([2H])c1[2H] ; c1ccc(-c2nc(-c3cccc(-n4c5ccccc5c5cc(-c6ccc7c(c6)c6ccccc6n7-c6ccccc6)ccc54)c3)nc3ccccc23)cc1]

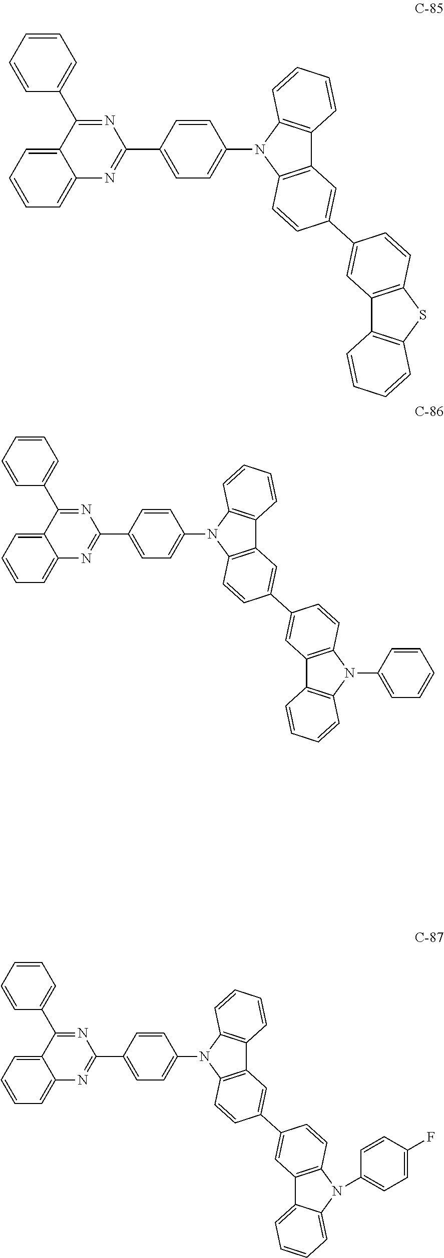 [SMILES: c1ccc(-c2nc(-c3ccc(-n4c5ccccc5c5cc(-c6ccc7sc8ccccc8c7c6)ccc54)cc3)nc3ccccc23)cc1 ; c1ccc(-c2nc(-c3ccc(-n4c5ccccc5c5cc(-c6ccc7c(c6)c6ccccc6n7-c6ccccc6)ccc54)cc3)nc3ccccc23)cc1 ; Fc1ccc(-n2c3ccccc3c3cc(-c4ccc5c(c4)c4ccccc4n5-c4ccc(-c5nc(-c6ccccc6)c6ccccc6n5)cc4)ccc32)cc1]

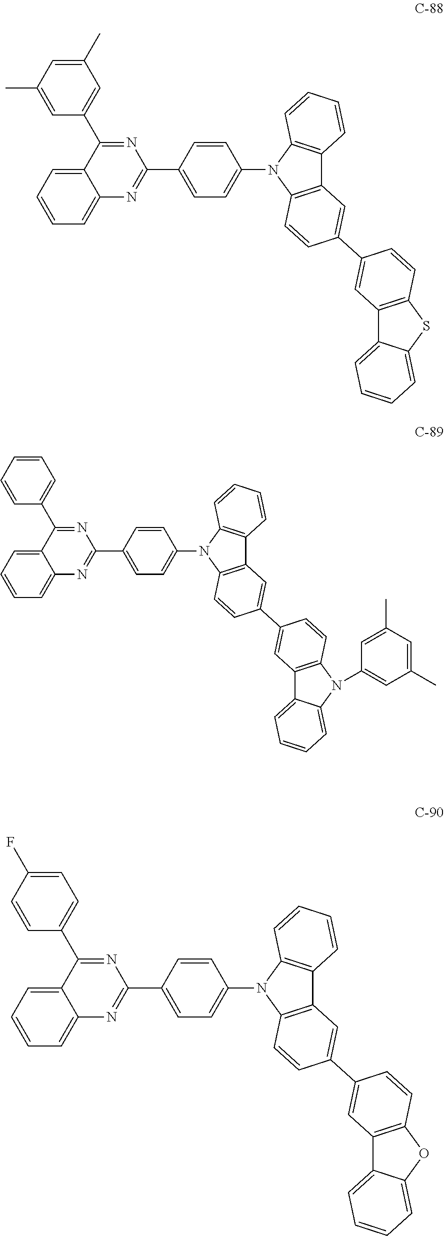 [SMILES: Cc1cc(C)cc(-c2nc(-c3ccc(-n4c5ccccc5c5cc(-c6ccc7sc8ccccc8c7c6)ccc54)cc3)nc3ccccc23)c1 ; Cc1cc(C)cc(-n2c3ccccc3c3cc(-c4ccc5c(c4)c4ccccc4n5-c4ccc(-c5nc(-c6ccccc6)c6ccccc6n5)cc4)ccc32)c1 ; Fc1ccc(-c2nc(-c3ccc(-n4c5ccccc5c5cc(-c6ccc7oc8ccccc8c7c6)ccc54)cc3)nc3ccccc23)cc1]

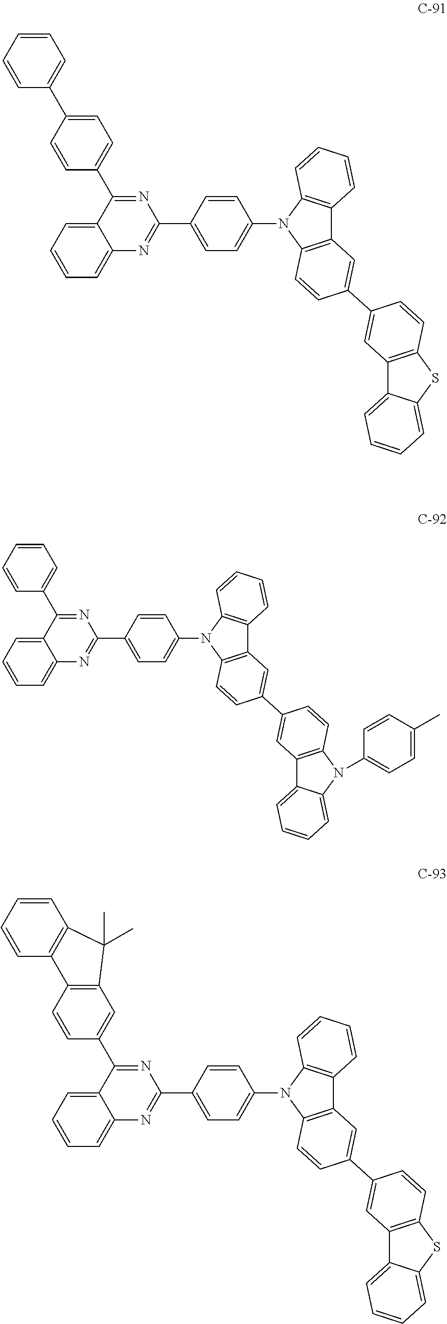 [SMILES: c1ccc(-c2ccc(-c3nc(-c4ccc(-n5c6ccccc6c6cc(-c7ccc8sc9ccccc9c8c7)ccc65)cc4)nc4ccccc34)cc2)cc1 ; Cc1ccc(-n2c3ccccc3c3cc(-c4ccc5c(c4)c4ccccc4n5-c4ccc(-c5nc(-c6ccccc6)c6ccccc6n5)cc4)ccc32)cc1 ; CC1(C)c2ccccc2-c2ccc(-c3nc(-c4ccc(-n5c6ccccc6c6cc(-c7ccc8sc9ccccc9c8c7)ccc65)cc4)nc4ccccc34)cc21]

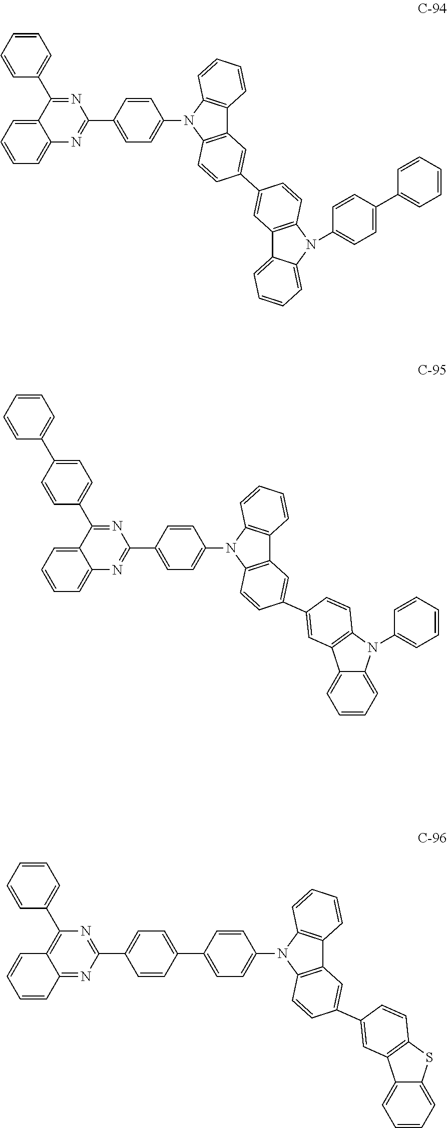 [SMILES: c1ccc(-c2ccc(-n3c4ccccc4c4cc(-c5ccc6c(c5)c5ccccc5n6-c5ccc(-c6nc(-c7ccccc7)c7ccccc7n6)cc5)ccc43)cc2)cc1 ; c1ccc(-c2ccc(-c3nc(-c4ccc(-n5c6ccccc6c6cc(-c7ccc8c(c7)c7ccccc7n8-c7ccccc7)ccc65)cc4)nc4ccccc34)cc2)cc1 ; c1ccc(-c2nc(-c3ccc(-c4ccc(-n5c6ccccc6c6cc(-c7ccc8sc9ccccc9c8c7)ccc65)cc4)cc3)nc3ccccc23)cc1]

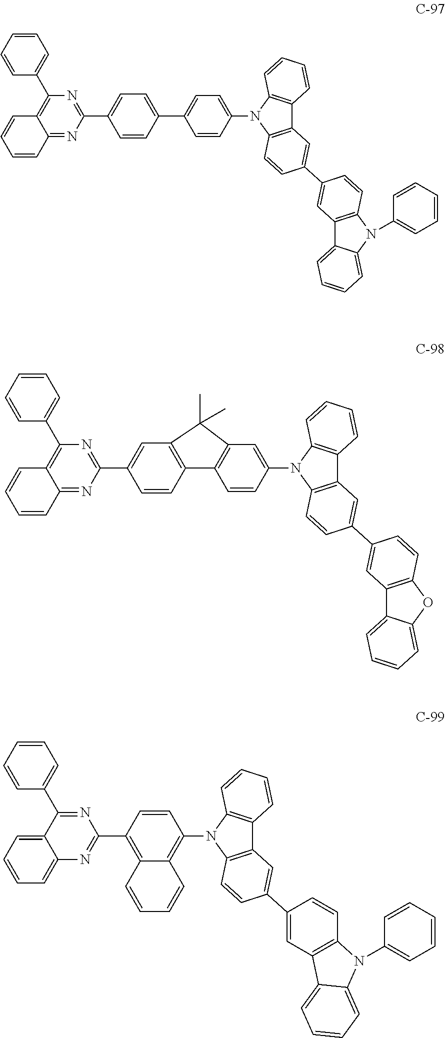 [SMILES: c1ccc(-c2nc(-c3ccc(-c4ccc(-n5c6ccccc6c6cc(-c7ccc8c(c7)c7ccccc7n8-c7ccccc7)ccc65)cc4)cc3)nc3ccccc23)cc1 ; CC1(C)c2cc(-c3nc(-c4ccccc4)c4ccccc4n3)ccc2-c2ccc(-n3c4ccccc4c4cc(-c5ccc6oc7ccccc7c6c5)ccc43)cc21 ; c1ccc(-c2nc(-c3ccc(-n4c5ccccc5c5cc(-c6ccc7c(c6)c6ccccc6n7-c6ccccc6)ccc54)c4ccccc34)nc3ccccc23)cc1]

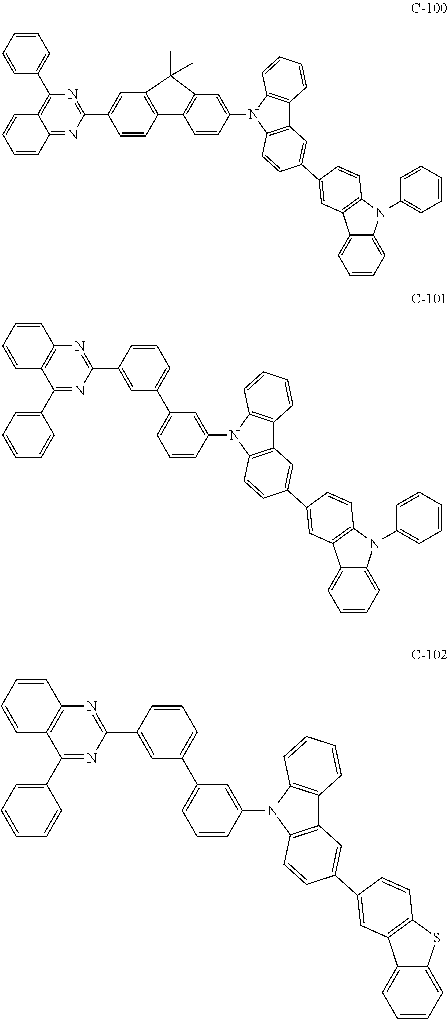

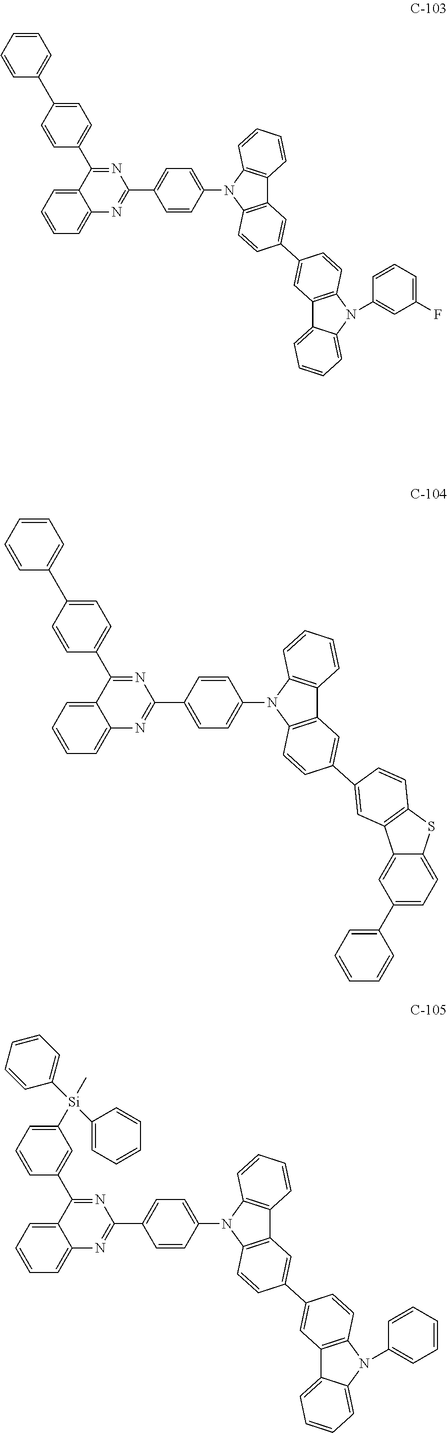 [SMILES: Fc1cccc(-n2c3ccccc3c3cc(-c4ccc5c(c4)c4ccccc4n5-c4ccc(-c5nc(-c6ccc(-c7ccccc7)cc6)c6ccccc6n5)cc4)ccc32)c1 ; c1ccc(-c2ccc(-c3nc(-c4ccc(-n5c6ccccc6c6cc(-c7ccc8sc9ccc(-c%10ccccc%10)cc9c8c7)ccc65)cc4)nc4ccccc34)cc2)cc1 ; C[Si](c1ccccc1)(c1ccccc1)c1cccc(-c2nc(-c3ccc(-n4c5ccccc5c5cc(-c6ccc7c(c6)c6ccccc6n7-c6ccccc6)ccc54)cc3)nc3ccccc23)c1]

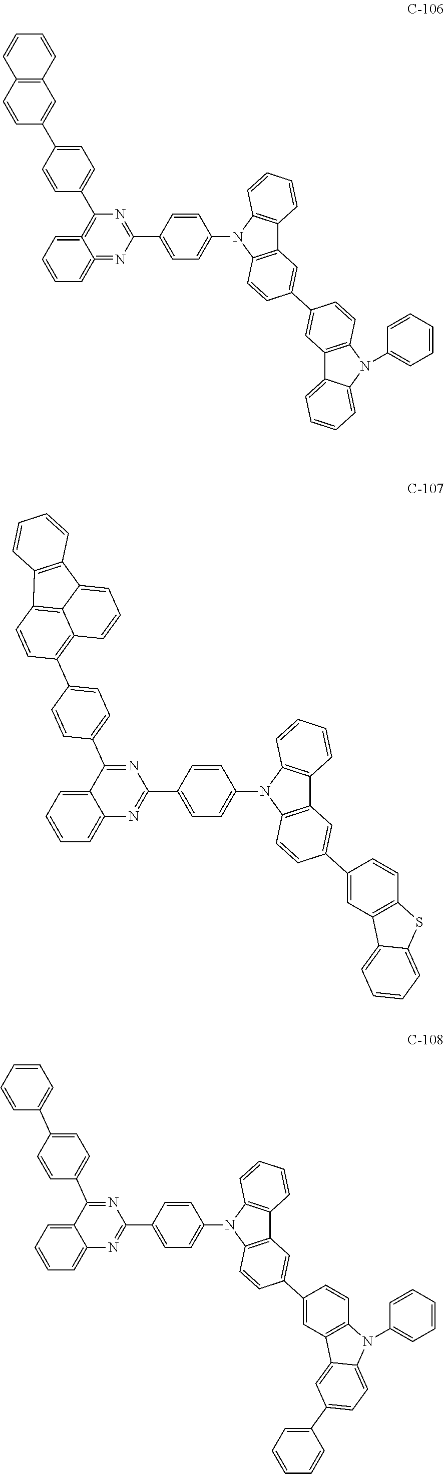

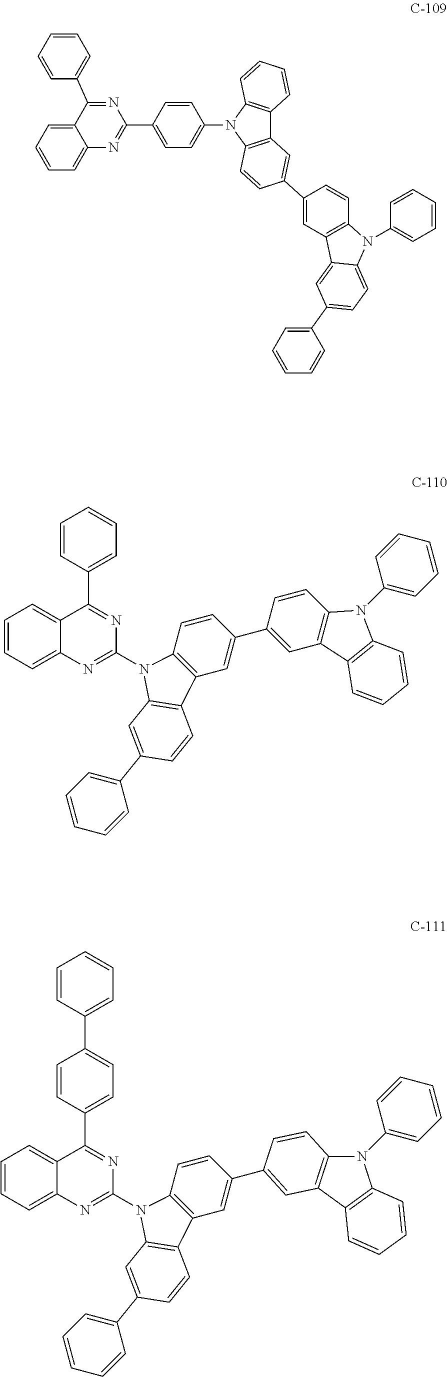 [SMILES: c1ccc(-c2ccc3c(c2)c2cc(-c4ccc5c(c4)c4ccccc4n5-c4ccc(-c5nc(-c6ccccc6)c6ccccc6n5)cc4)ccc2n3-c2ccccc2)cc1 ; c1ccc(-c2ccc3c4cc(-c5ccc6c(c5)c5ccccc5n6-c5ccccc5)ccc4n(-c4nc(-c5ccccc5)c5ccccc5n4)c3c2)cc1 ; c1ccc(-c2ccc(-c3nc(-n4c5ccc(-c6ccc7c(c6)c6ccccc6n7-c6ccccc6)cc5c5ccc(-c6ccccc6)cc54)nc4ccccc34)cc2)cc1]

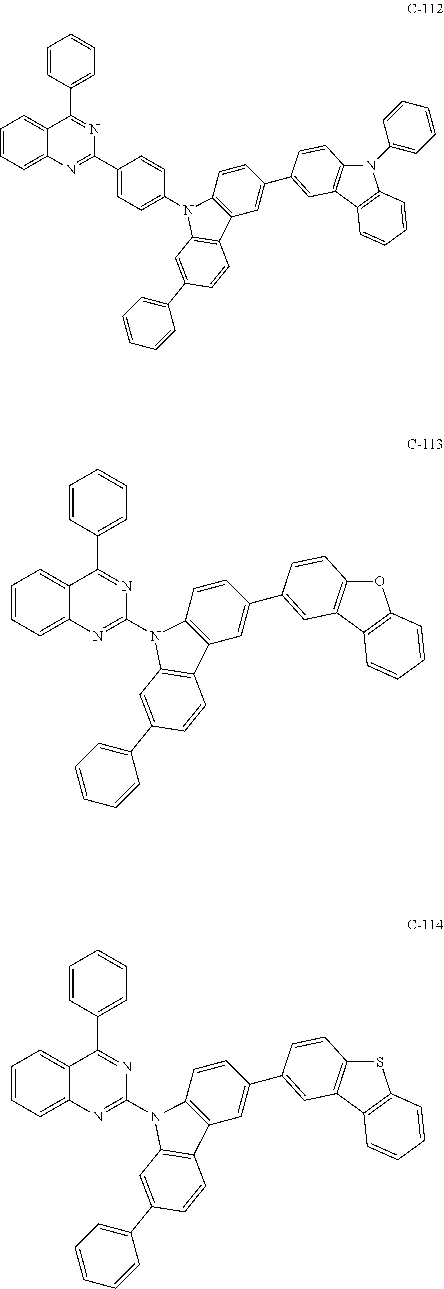 [SMILES: c1ccc(-c2ccc3c4cc(-c5ccc6c(c5)c5ccccc5n6-c5ccccc5)ccc4n(-c4ccc(-c5nc(-c6ccccc6)c6ccccc6n5)cc4)c3c2)cc1 ; c1ccc(-c2ccc3c4cc(-c5ccc6oc7ccccc7c6c5)ccc4n(-c4nc(-c5ccccc5)c5ccccc5n4)c3c2)cc1 ; c1ccc(-c2ccc3c4cc(-c5ccc6sc7ccccc7c6c5)ccc4n(-c4nc(-c5ccccc5)c5ccccc5n4)c3c2)cc1]

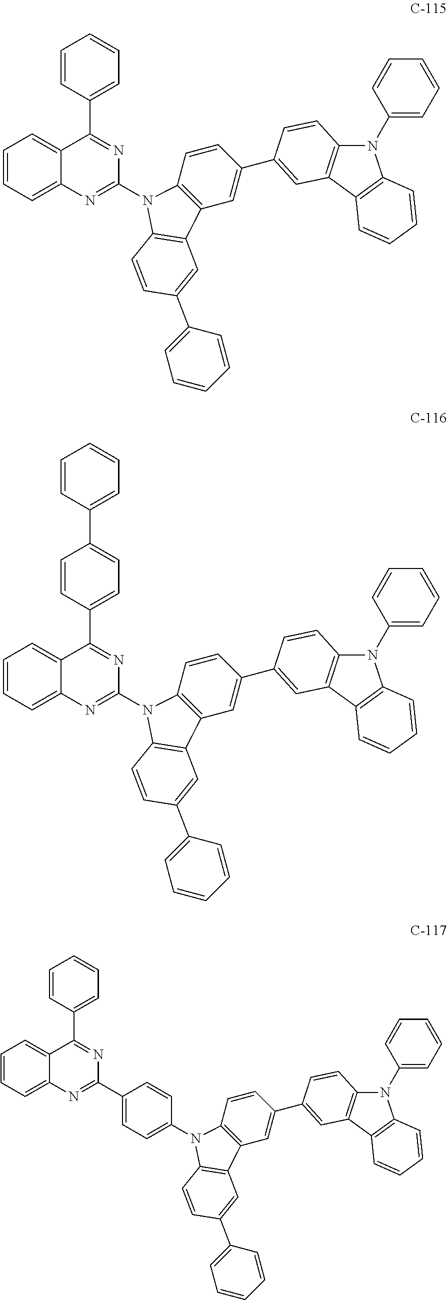 [SMILES: c1ccc(-c2ccc3c(c2)c2cc(-c4ccc5c(c4)c4ccccc4n5-c4ccccc4)ccc2n3-c2nc(-c3ccccc3)c3ccccc3n2)cc1 ; c1ccc(-c2ccc(-c3nc(-n4c5ccc(-c6ccccc6)cc5c5cc(-c6ccc7c(c6)c6ccccc6n7-c6ccccc6)ccc54)nc4ccccc34)cc2)cc1 ; c1ccc(-c2ccc3c(c2)c2cc(-c4ccc5c(c4)c4ccccc4n5-c4ccccc4)ccc2n3-c2ccc(-c3nc(-c4ccccc4)c4ccccc4n3)cc2)cc1]

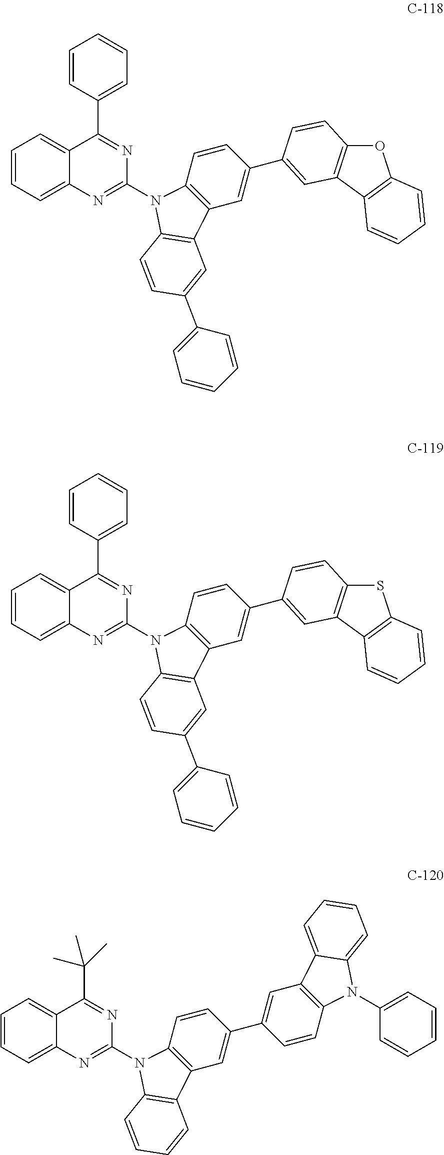 [SMILES: c1ccc(-c2ccc3c(c2)c2cc(-c4ccc5oc6ccccc6c5c4)ccc2n3-c2nc(-c3ccccc3)c3ccccc3n2)cc1 ; c1ccc(-c2ccc3c(c2)c2cc(-c4ccc5sc6ccccc6c5c4)ccc2n3-c2nc(-c3ccccc3)c3ccccc3n2)cc1 ; CC(C)(C)c1nc(-n2c3ccccc3c3cc(-c4ccc5c(c4)c4ccccc4n5-c4ccccc4)ccc32)nc2ccccc12]

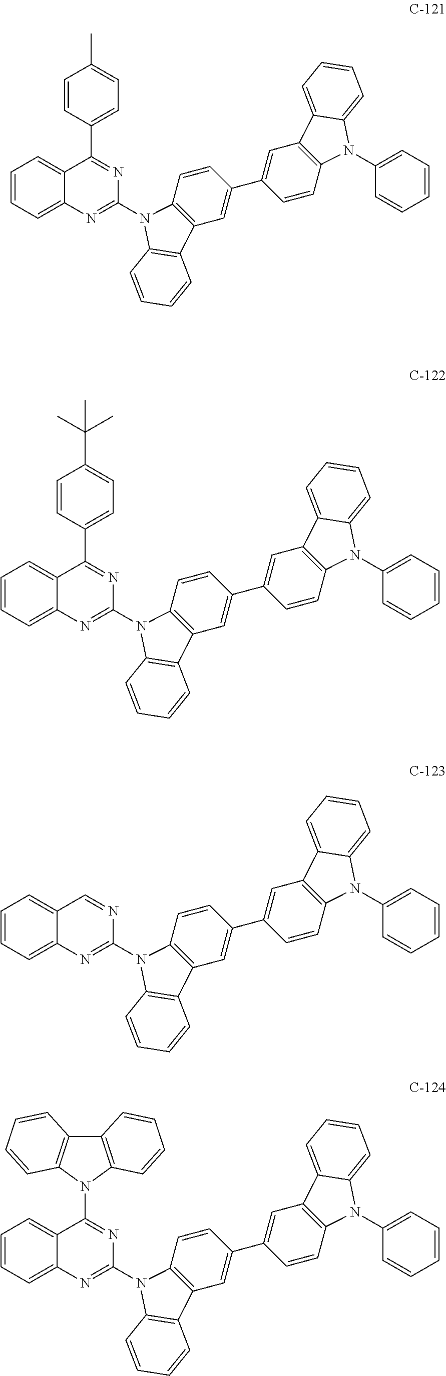 [SMILES: Cc1ccc(-c2nc(-n3c4ccccc4c4cc(-c5ccc6c(c5)c5ccccc5n6-c5ccccc5)ccc43)nc3ccccc23)cc1 ; CC(C)(C)c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3cc(-c4ccc5c(c4)c4ccccc4n5-c4ncc5ccccc5n4)ccc32)cc1 ; c1ccc(-n2c3ccccc3c3cc(-c4ccc5c(c4)c4ccccc4n5-c4nc(-n5c6ccccc6c6ccccc65)c5ccccc5n4)ccc32)cc1]

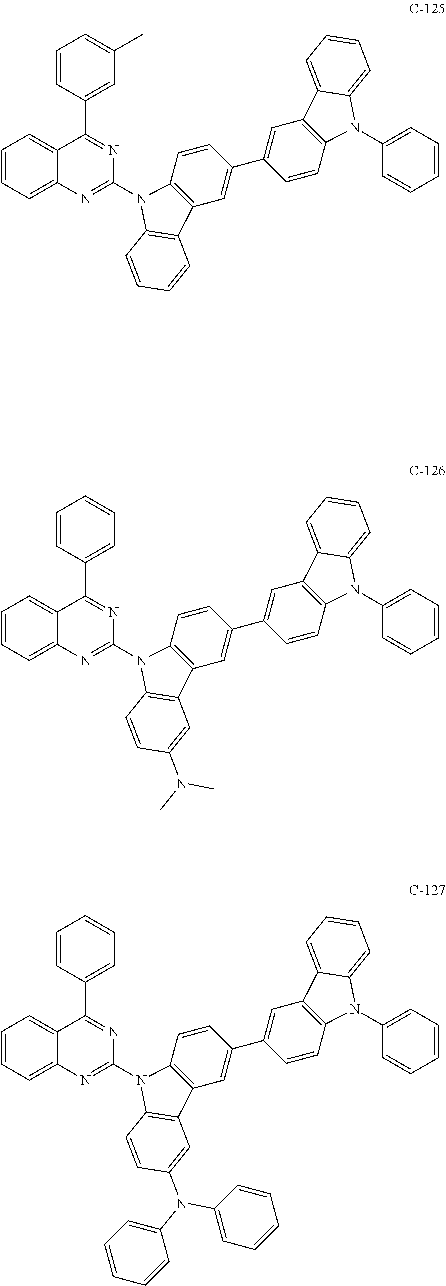 [SMILES: Cc1cccc(-c2nc(-n3c4ccccc4c4cc(-c5ccc6c(c5)c5ccccc5n6-c5ccccc5)ccc43)nc3ccccc23)c1 ; CN(C)c1ccc2c(c1)c1cc(-c3ccc4c(c3)c3ccccc3n4-c3ccccc3)ccc1n2-c1nc(-c2ccccc2)c2ccccc2n1 ; c1ccc(-c2nc(-n3c4ccc(-c5ccc6c(c5)c5ccccc5n6-c5ccccc5)cc4c4cc(N(c5ccccc5)c5ccccc5)ccc43)nc3ccccc23)cc1]

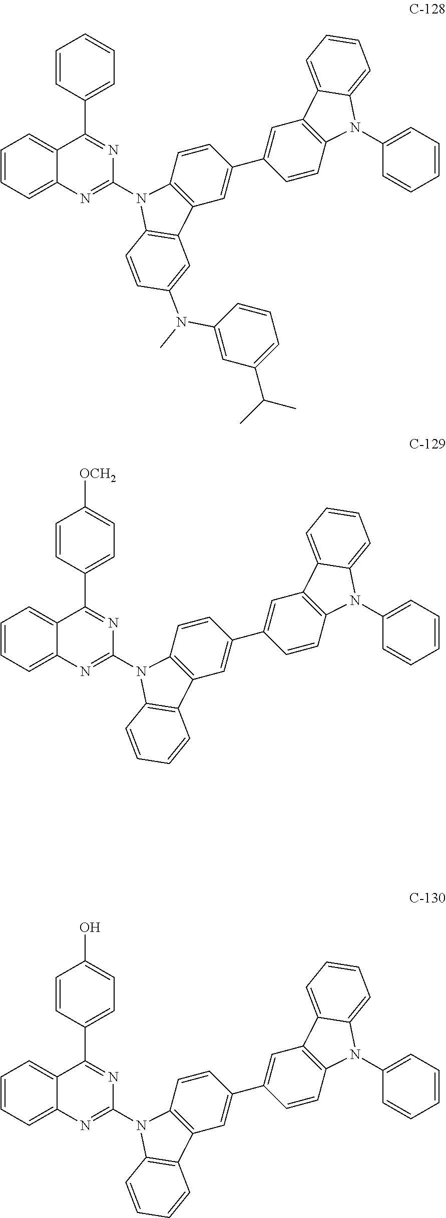

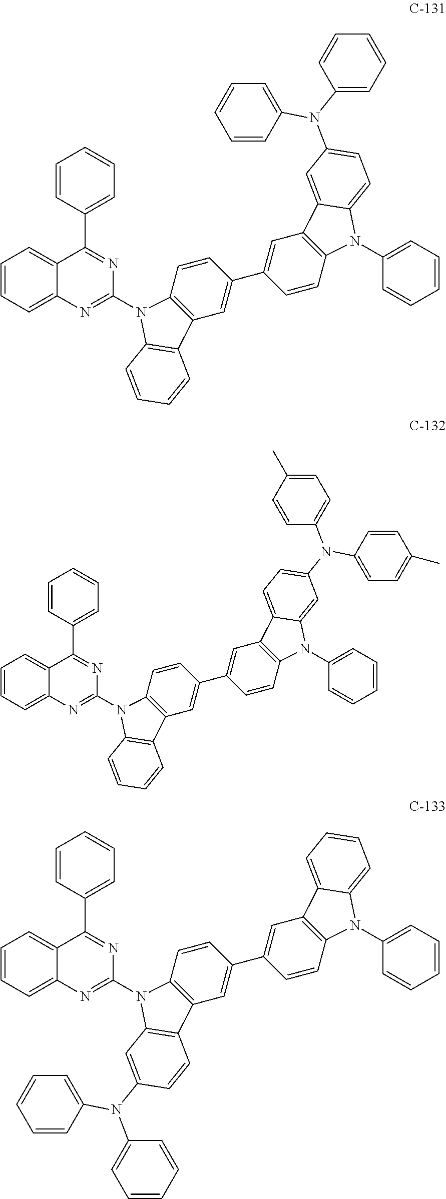 [SMILES: c1ccc(-c2nc(-n3c4ccccc4c4cc(-c5ccc6c(c5)c5cc(N(c7ccccc7)c7ccccc7)ccc5n6-c5ccccc5)ccc43)nc3ccccc23)cc1 ; Cc1ccc(N(c2ccc(C)cc2)c2ccc3c4cc(-c5ccc6c(c5)c5ccccc5n6-c5nc(-c6ccccc6)c6ccccc6n5)ccc4n(-c4ccccc4)c3c2)cc1 ; c1ccc(-c2nc(-n3c4ccc(-c5ccc6c(c5)c5ccccc5n6-c5ccccc5)cc4c4ccc(N(c5ccccc5)c5ccccc5)cc43)nc3ccccc23)cc1]

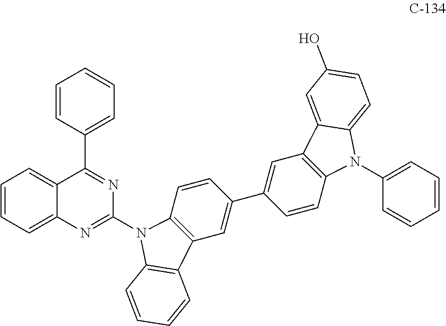 [SMILES: Oc1ccc2c(c1)c1cc(-c3ccc4c(c3)c3ccccc3n4-c3nc(-c4ccccc4)c4ccccc4n3)ccc1n2-c1ccccc1]

Organic electroluminescent compounds according to the present invention can be prepared by well-known methods in the art, for example, according to the following scheme 1.

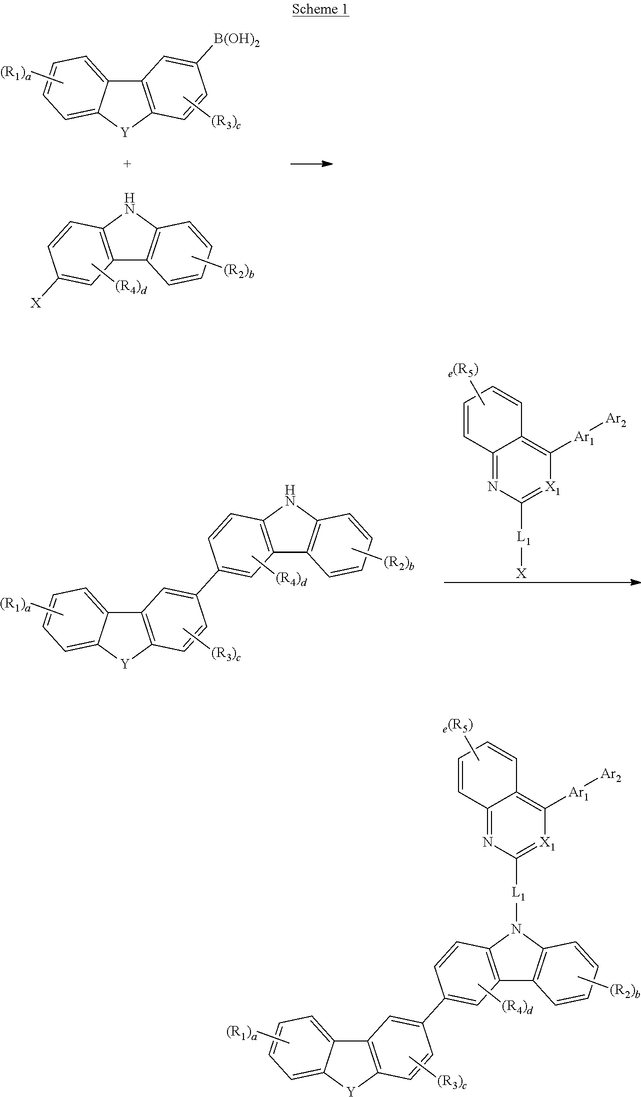

wherein R₁ to R₅, Ar₁, Ar₂, Y, X₁, L₁, a, b, c, d and e are as defined in formula 1 above, and X represents a halogen.

Further, the present invention provides an organic electroluminescent device comprising the organic electroluminescent compound of formula 1.

Said organic electroluminescent device comprises a first electrode, a second electrode and at least one organic layer between said first electrode and said second electrode. Said organic layer comprises at least one organic electroluminescent compound of formula 1. Further, said organic layer comprises a light-emitting layer in which the organic electroluminescent compound of formula 1 is comprised as a host material. Where the organic electroluminescent compound of formula 1 is comprised as a host material in the light-emitting layer, said light-emitting layer further comprises at least one phosphorescent dopant. In the organic electroluminescent device of the present invention, said phosphorescent dopant is not particularly limited, but may be selected from compounds represented by the following formula 2:

M¹L¹⁰¹L¹⁰²L¹⁰³ Formula 2

wherein

M¹ is selected from the group consisting of Ir, Pt, Pd and Os; L¹⁰¹, L¹⁰² and L¹⁰³ each independently are selected from the following structures:

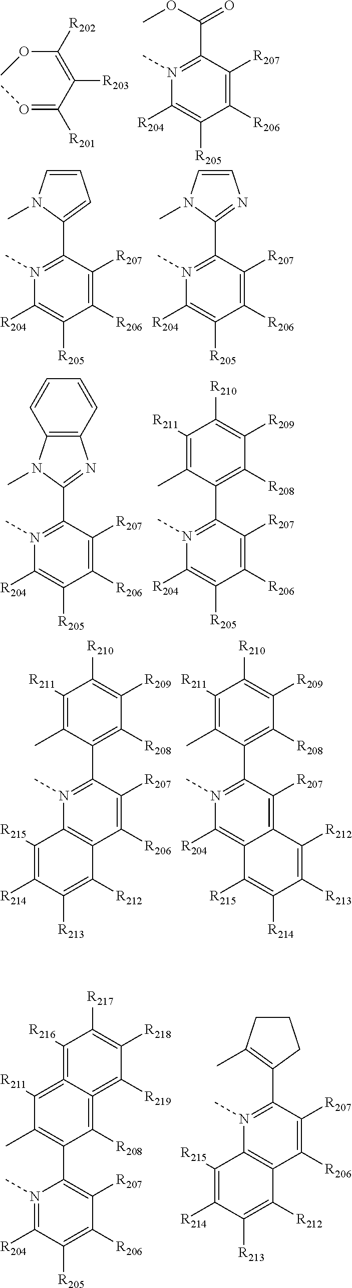

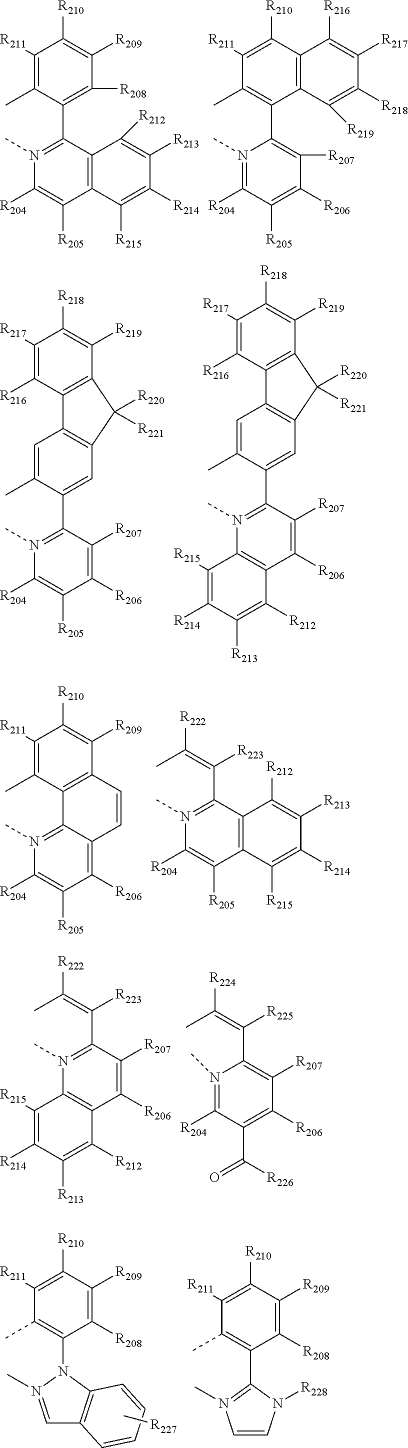

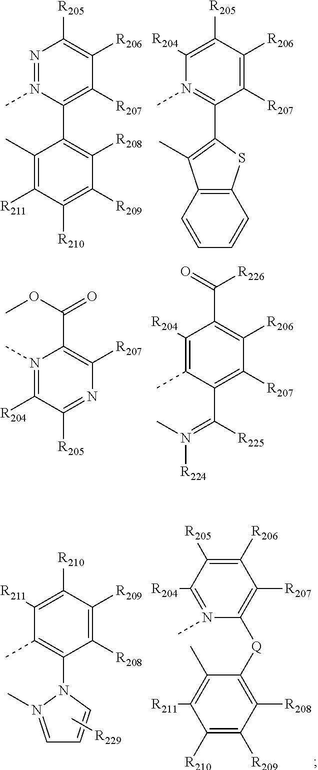

R₂₀₁ to R₂₀₃ each independently represent hydrogen, deuterium, a (C1-C30)alkyl group unsubstituted or substituted by a halogen(s), a (C6-C30)aryl group unsubstituted or substituted by a (C1-C30)alkyl group(s), or a halogen; R₂₀₄ to R₂₁₉ each independently represent hydrogen, deuterium, a substituted or unsubstituted (C1-C30)alkyl group, a substituted or unsubstituted (C1-C30)alkoxy group, a substituted or unsubstituted (C3-C30)cycloalkyl group, a substituted or unsubstituted (C2-C30)alkenyl group, a substituted or unsubstituted (C6-C30)aryl group, a substituted or unsubstituted mono- or di-(C1-C30)alkylamino group, a substituted or unsubstituted mono- or di-(C6-C30)arylamino group, SF₅, a substituted or unsubstituted tri(C1-C30)alkylsilyl group, a substituted or unsubstituted di(C1-C30)alkyl(C6-C30)arylsilyl group, a substituted or unsubstituted tri(C6-C30)arylsilyl group, a cyano group or a halogen; R₂₂₀ to R₂₂₃ each independently represent hydrogen, deuterium, a (C1-C30)alkyl group unsubstituted or substituted by a halogen(s), or a (C6-C30)aryl group unsubstituted or substituted by a (C1-C30)alkyl group(s); R₂₂₄ and R₂₂₅ each independently represent hydrogen, deuterium, a substituted or unsubstituted (C1-C30)alkyl group, a substituted or unsubstituted (C6-C30)aryl group, or a halogen, or R₂₂₄ and R₂₂₅ may be linked to each other via a (C3-C12)alkylene group or (C3-C12)alkenylene group with or without a fused ring, to form a mono- or polycyclic alicyclic ring or a mono- or polycyclic aromatic ring; R₂₂₆ represents a substituted or unsubstituted (C1-C30)alkyl group, a substituted or unsubstituted (C6-C30)aryl group, a substituted or unsubstituted 5- or 30-membered heteroaryl group or a halogen; R₂₂₇ to R₂₂₉ each independently represent hydrogen, deuterium, a substituted or unsubstituted (C1-C30)alkyl group, a substituted or unsubstituted (C6-C30)aryl group or a halogen; Q represents,

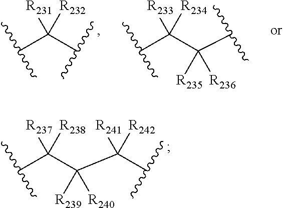

R₂₃₁ to R₂₄₂ each independently represent hydrogen, deuterium, a (C1-C30)alkyl group unsubstituted or substituted by a halogen(s), a (C1-C30)alkoxy group, a halogen, a substituted or unsubstituted (C6-C30)aryl group, a cyano group, a substituted or unsubstituted (C5-C30)cycloalkyl group, or each of R₂₃₁ to R₂₄₂ may be linked to an adjacent substituent via (C2-C30) alkylene group or (C2-C30)alkenylene group to form a spiro ring or a fused ring or may be linked to R₂₀₇ or R₂₀₈ via (C2-C30) alkylene group or (C2-C30)alkenylene group to form a saturated or unsaturated fused ring.

The dopants of formula 2 include the following, but are not limited thereto:

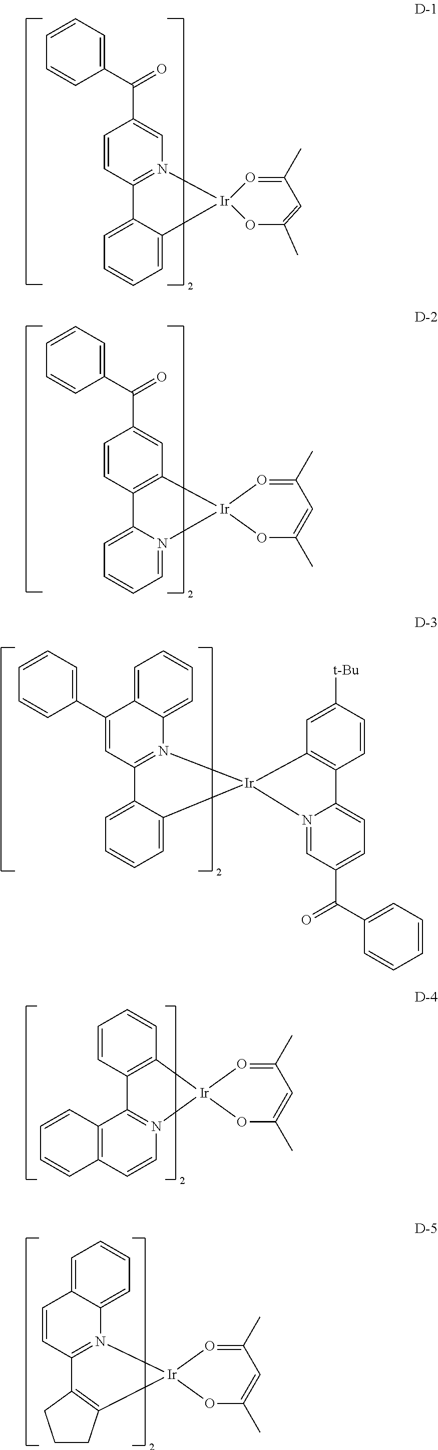

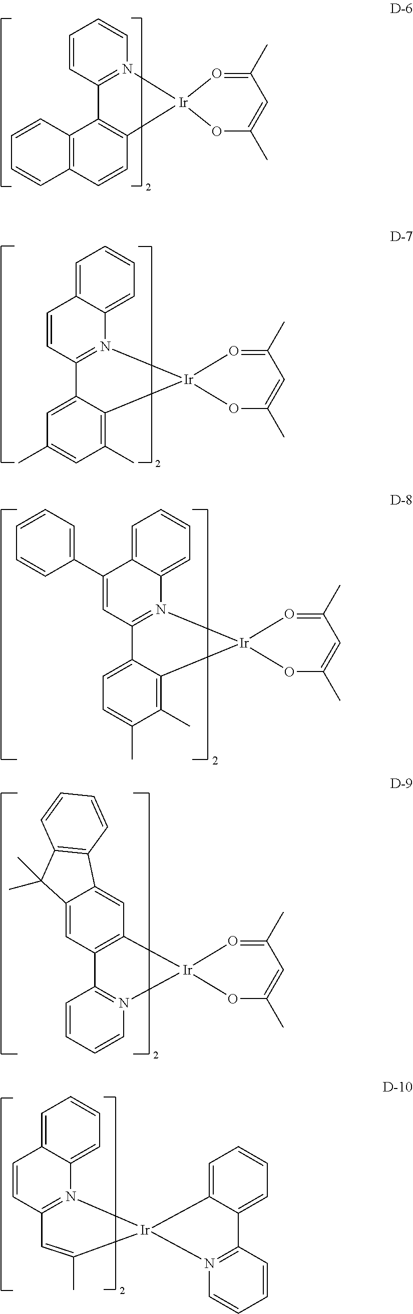

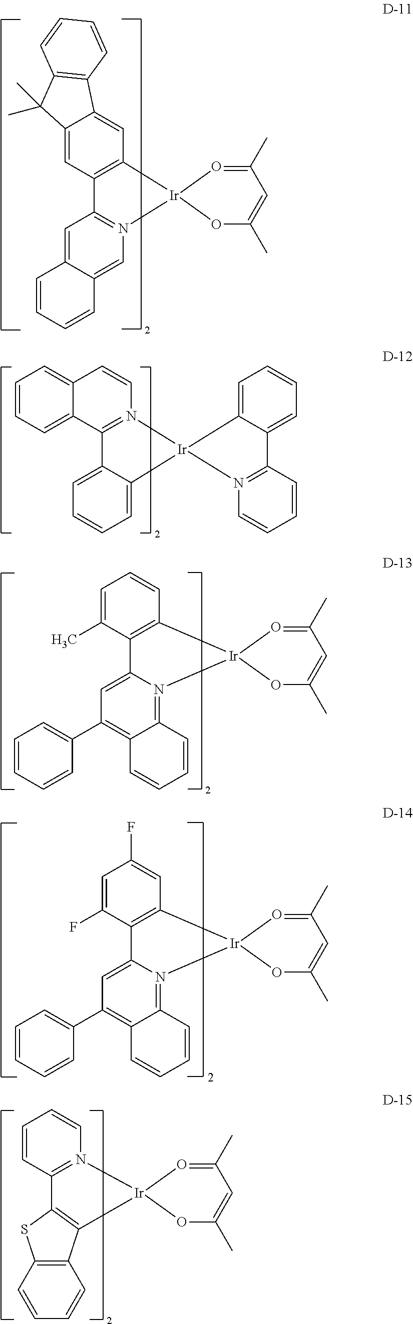

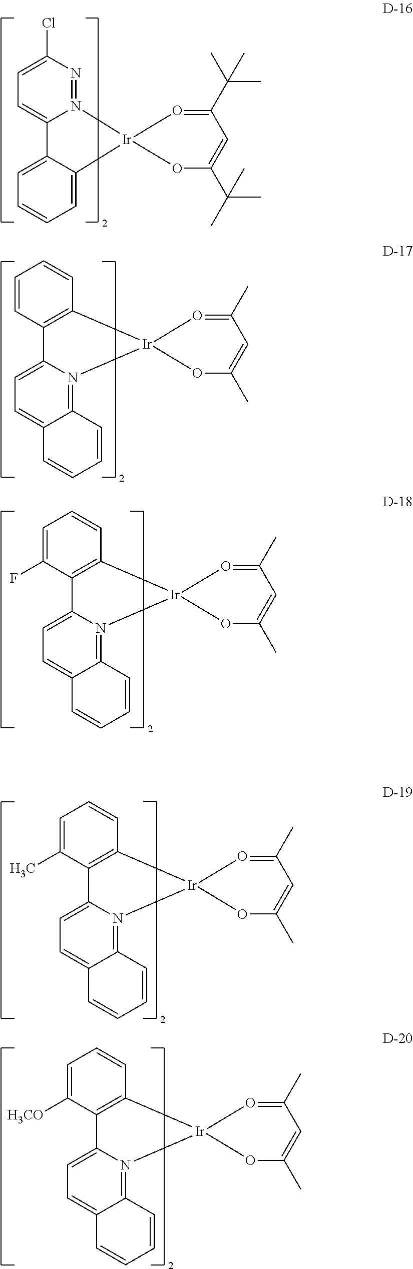

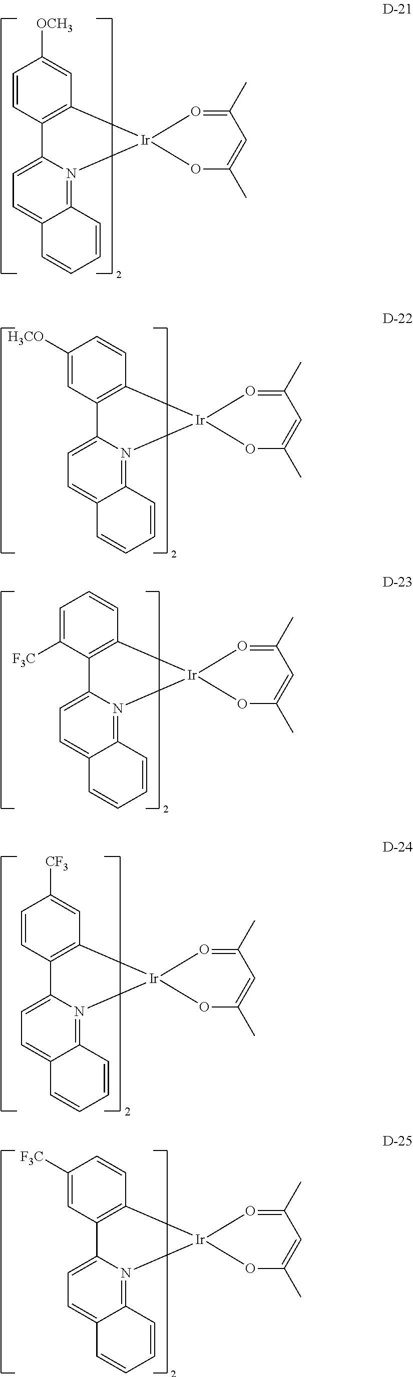

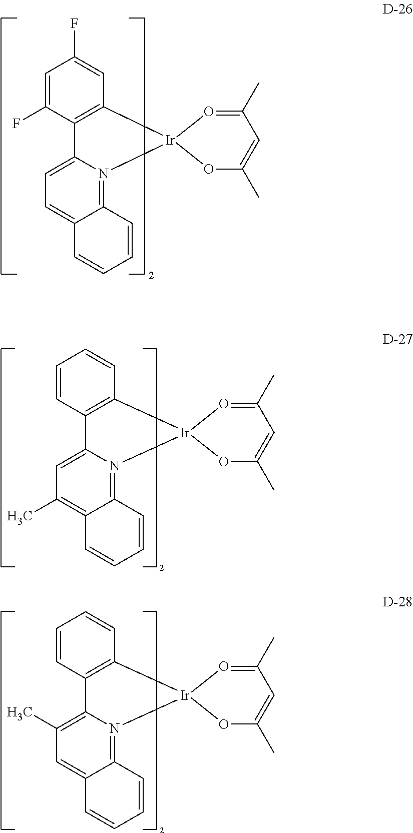

The organic electroluminescent device according to the present invention may further comprise, in addition to the organic electroluminescent compound according to the present invention, at least one amine-based compound selected from the group consisting of arylamine-based compounds and styrylarylamine-based compounds.

In the organic electroluminescent device according to the present invention, the organic layer may further comprise at least one metal selected from the group consisting of metals of Group 1, metals of Group 2, transition metals of the 4^{th} period, transition metals of the 5^{th} period, lanthanides and organic metals of d-transition elements of the Periodic Table, or at least one complex compound comprising said metal. The organic layer may comprise a light-emitting layer and a charge generating layer.

The organic electroluminescent device according to the present invention may emit a white light by further comprising in addition to the organic electroluminescent compound according to the present invention, at least one light-emitting layer which comprises a blue electroluminescent compound, a red electroluminescent compound or a green electroluminescent compound. If necessary, the organic electroluminescent device may further comprise a yellow light-emitting layer or an orange light-emitting layer.

Preferably, in the organic electroluminescent device according to the present invention, at least one layer (hereinafter, “a surface layer”) selected from a chalcogenide layer, a metal halide layer and a metal oxide layer may be placed on an inner surface(s) of one or both electrode(s). Specifically, it is preferred that a chalcogenide layer of silicon or aluminum is placed on an anode surface of an electroluminescent medium layer, and a metal halide layer or metal oxide layer is placed on a cathode surface of an electroluminescent medium layer. Such a surface layer provides operation stability for the organic electroluminescent device. Preferably, said chalcogenide includes SiO_{X}(1≦X≦2), AlO_{X}(1≦X≦1.5), SiON, SiAlON, etc.; said metal halide includes LiF, MgF₂, CaF₂, a rare earth metal fluoride, etc.; and said metal oxide includes Cs₂O, Li₂O, MgO, SrO, BaO, CaO, etc.

Preferably, in the organic electroluminescent device according to the present invention, a mixed region of an electron transport compound or a mixed region of a hole transport compound and an oxidative dopant may be placed on at least one surface of a pair of electrodes. In that case, the electron transport compound is reduced to an anion, and thus facilitates injecting and transporting electrons to an electroluminescent medium. Further, the hole transport compound is oxidized to a cation, and thus facilitates injecting and transporting holes to an electroluminescent medium. Preferably, the oxidative dopant includes various Lewis acids and acceptor compounds; and the reductive dopant includes alkali metals, alkali metal compounds, alkaline earth metals, rare-earth metals, and mixtures thereof. A reductive dopant layer may be employed as a charge generating layer to prepare an electroluminescent device having two or more electroluminescent layers and emitting a white light.

### Advantageous Effects of the Invention

The organic electroluminescent compound according to the present invention provides an organic electroluminescent device which has high luminous efficiency and a long operation lifetime and requires a low driving voltage improving power efficiency and power consumption.

### MODE FOR THE INVENTION

Hereinafter, examples are provided for preparing the organic electroluminescent compounds, and properties of the organic electroluminescent devices using them.

The abbreviations used in the examples have the following meanings:

Ph: phenyl, MeOH: methanol, EtOH: ethanol, MC: methylene chloride, EA: ethyl acetate,

DMF: dimethylformamide, n-Bu: normal-butyl, i-Pr: isopropyl, Me: methyl,

THF: tetrahydrofuran, EDA: ethylene diamine, NBS: N-bromosuccinimide

### Preparation Example 1

### Preparation of Compound C-3

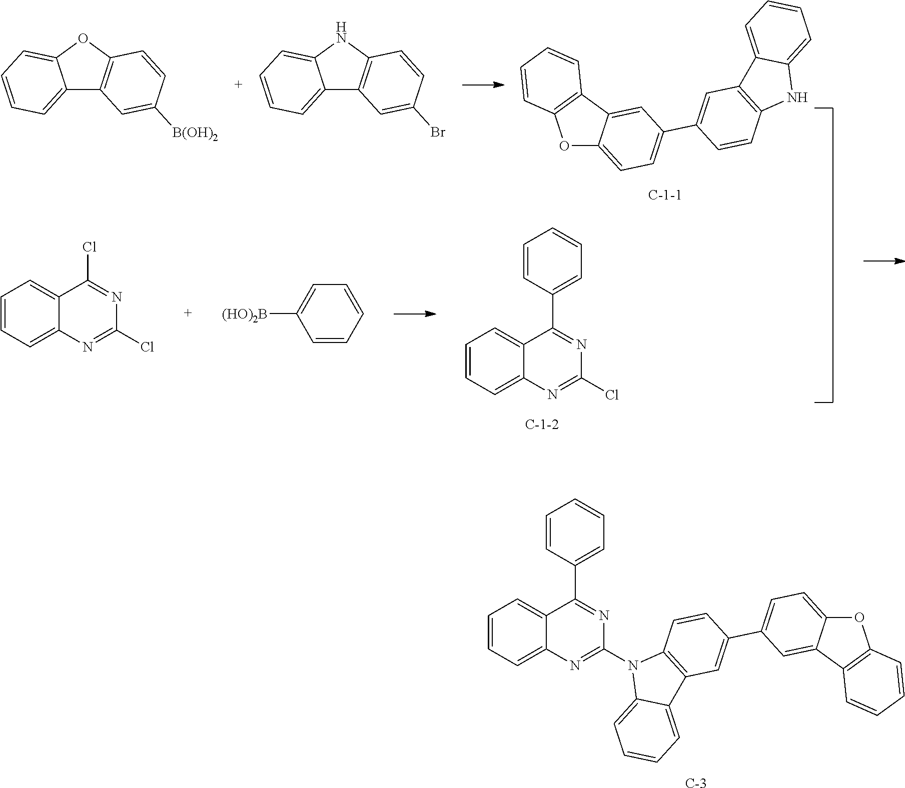

### Preparation of Compound C-1-1

Dibenzo[b,d]furan-2-yl boronic acid (10.33 g, 48.76 mmol), 3-bromo-9H-carbazole (10 g, 40.63 mmol), K₂CO₃ (13.5 g, 97.52 mmol) and Pd(PPh₃)₄ (2.35 g, 2.03 mmol) were added to toluene 200 mL, EtOH 50 mL and purified water 50 mL. After stirring the reaction mixture for 3 hours at 90 to 100° C., the mixture was cooled to room temperature. An aqueous layer was removed from the mixture by a gravity separation. The obtained organic layer was concentrated, was triturated with MC, and then was filtered to obtain compound C-1-1 (9.75 g, 72%).

### Preparation of Compound C-1-2

After dissolving 2,4-dichloroquinazoline (30 g, 151 mmol), phenylboronic acid (9.2 g, 75.3 mmol), Pd(PPh₃)₄ (2.6 g, 2.3 mmol) and Na₂CO₃ (16 g, 150 mmol) in toluene (300 mL) and distilled water (75 mL), the reaction mixture was stirred for 2 hours at 90° C. The mixture was distillated under reduced pressure to obtain an organic layer, and then was triturated with MeOH. The obtained solid was dissolved in MC, was filtered through silica, and then was triturated with MC and hexane to obtain compound C-1-2 (9.3 g, 51.4%).

### Preparation of Compound C-3

After suspending compound C-1-1 (5.3 g, 14.7 mmol) and compound C-1-2 (5 g, 15.8 mmol) in DMF 80 mL. 60% NaH (948 mg, 22 mmol) was added to the mixture at room temperature. The obtained reaction mixture was stirred for 12 hours. After adding purified water (1 L), the mixture was filtered under reduced pressure. The obtained solid was triturated with MeOH/EA, was dissolved in MC, was filtered through silica, and then was triturated with MC/n-hexane to obtain compound C-3 (5 g, 51.5%).

### Preparation Example 2

### Preparation of Compound C-9

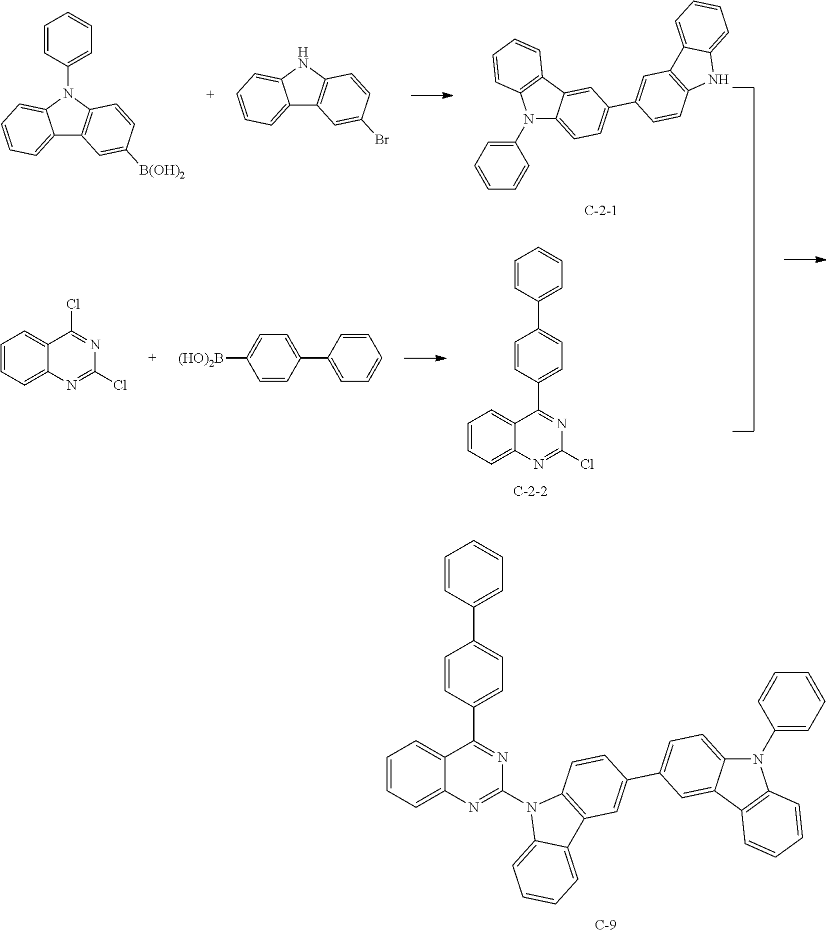

### Preparation of Compound C-2-1

9-phenyl-9H-carbazol-3-yl boronic acid (14 g, 48.76 mmol), 3-bromo-9H-carbazole (10 g, 40.63 mmol), K₂CO₃ (13.5 g, 97.52 mmol) and Pd(PPh₃)₄ (2.35 g, 2.03 mmol) were added to toluene 200 mL, EtOH 50 mL and purified water 50 mL. After stirring the reaction mixture for 3 hours at 90 to 100° C., the mixture was cooled to room temperature. An aqueous layer was removed from the mixture by a gravity separation. The obtained organic layer was concentrated, was triturated with MC, and then was filtered to obtain compound C-2-1 (12 g, 72%).

### Preparation of Compound C-2-2

2,4-dichloroquinazoline (20 g, 0.1 mol), biphenyl-4-yl boronic acid (18.9 g, 0.1 mol), Pd(PPh₃)₄ (3.5 g, 3.01 mmol) and Na₂CO₃ (31.9 g, 0.3 mol) were added to toluene 800 mL, EtOH 200 mL and purified water 200 mL. After stirring the reaction mixture for 3 hours at 70 to 80° C., an aqueous layer was removed from the mixture by a gravity separation. The obtained organic layer was concentrated, and then was purified by silica column chromatography to obtain compound C-2-2 (15 g, 47%).

### Preparation of Compound C-9

After suspending compound C-2-2 (4.6 g, 14.7 mmol) and compound C-2-1 (5 g, 12.2 mmol) in DMF 80 mL, 60% NaH (881 g, 22 mmol) was added to the mixture at room temperature. The obtained reaction mixture was stirred for 12 hours. After adding purified water (1 L), the mixture was filtered under reduced pressure. The obtained solid was triturated with MeOH/EA, was dissolved in MC, was filtered through silica, and then was triturated with MC/n-hexane to obtain compound C-9 (4 g, 47.4%).

### Preparation Example 3

### Preparation of Compound C-12

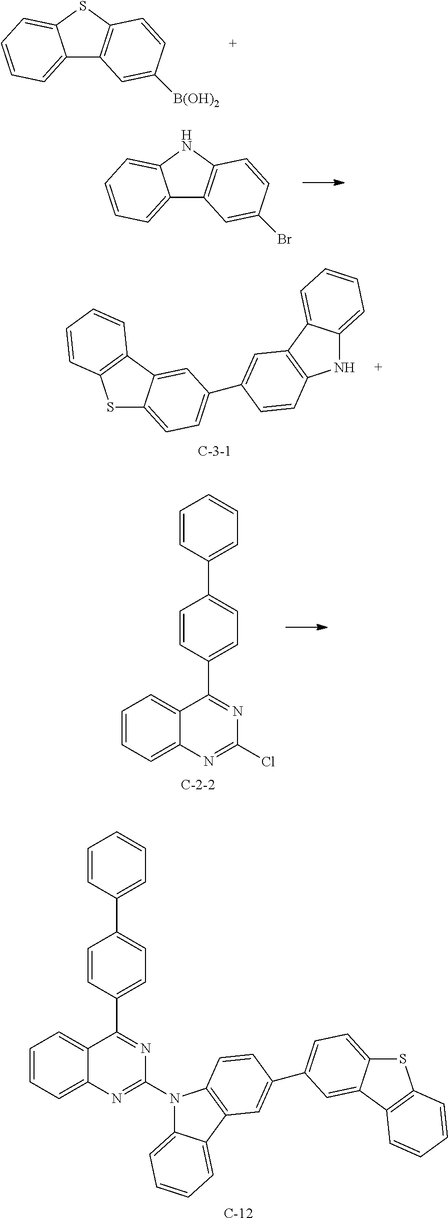

### Preparation of Compound C-3-1

Dibenzo[b,d]thiophen-2-yl boronic acid (10.33 g, 48.76 mmol), 3-bromo-9H-carbazole (10 g, 40.63 mmol), K₂CO₃ (13.5 g, 97.52 mmol), and Pd(PPh₃)₄ (2.35 g, 2.03 mmol) were added to toluene 200 mL, EtOH 50 mL and purified water 50 mL. After stirring the reaction mixture for 3 hours at 90 to 100° C., the mixture was cooled to room temperature. An aqueous layer was removed from the mixture by a gravity separation. The obtained organic layer was concentrated, was triturated with MC, and then was filtered to obtain compound C-3-1 (9.75 g, 72%).

### Preparation of Compound C-12

After suspending compound C-3-1 (5.5 g, 15.8 mmol) and compound C-2-2 (5 g, 15.8 mmol) in DMF 80 mL, 60% NaH (948 mg, 22 mmol) was added to the mixture at room temperature. The obtained reaction mixture was stirred for 12 hours. After adding purified water (1 L), the mixture was filtered under reduced pressure. The obtained solid was triturated with MeOH/EA, was dissolved in MC, was filtered through silica, and then was triturated with MC/n-hexane. Compound C-12 (5.2 g, 52%) was obtained.

### Preparation Example 4

### Preparation of Compound C-15

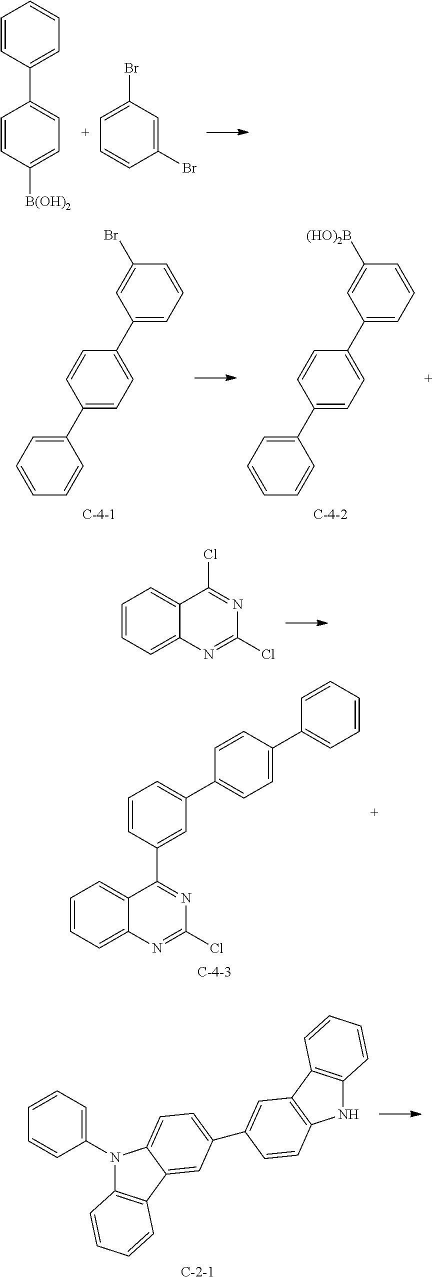

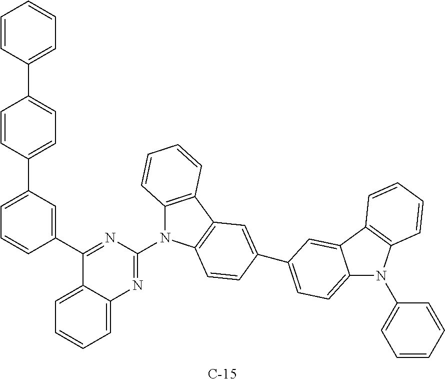

### Preparation of Compound C-4-1

After dissolving biphenyl-4-yl boronic acid (157 g, 554 mmol), 1,3-dibromobenzene (100 g, 581.7 mmol), Pd(PPh₃)₄ (13 g, 11.08 mmol) and Na₂CO₃ (150 g, 1.385 mol) in toluene (3.5 L), EtOH (0.7 L) and distilled water (0.7 L), the reaction mixture was stirred for 3 hours at 90° C. The mixture was extracted with EA and distilled water, was dissolved in chloroform (10 L) by heat, and then was filtered through silica. After triturating the resultant with EA and hexane, the resultant was triturated with EA and MeOH to obtain compound C-4-1 (94 g, 60%).

### Preparation of Compound C-4-2

After dissolving compound C-4-1 (55 g, 178 mmol) in THF (800 mL), 2.5 M n-BuLi in hexane (106 mL, 267 mmol) was added to the reaction mixture at −78° C., and then the mixture was stirred for 1 hour. B(Oi-Pr)₃ (82 mL, 356 mmol) was added slowly to the mixture, and then the mixture was stirred for 2 hours. The mixture was quenched by adding 2 M HCl, was extracted with distilled water and EA, and then was recrystallized with hexane and acetone. Compound C-4-2 (43 g, 88.0%) was obtained.

### Preparation of Compound C-4-3

2,4-dichloroquinazoline (20 g, 73 mmol), compound C-4-2 (15 g, 73 mmol), Pd(PPh₃)₄ (2.5 g, 2.2 mmol) and Na₂CO₃ (23 g, 241 mmol) were dissolved in toluene (500 mL), EtOH (100 mL) and distilled water (100 mL), and then was stirred for 5 hours at 100° C. The reaction mixture was distillated under reduced pressure to obtain an organic layer, and then was triturated with MeOH. The obtained solid was dissolved in MC, was filtered through silica, and then was triturated with MC and hexane to obtain compound C-4-3 (19.5 g, 68%).

### Preparation of Compound C-15

After suspending compound C-2-1 (5 g, 12.2 mmol) and compound C-4-3 (4.6 g, 11.6 mmol) in DMF 80 mL, 60% NaH (881 mg, 22 mmol) was added to the mixture at room temperature. The obtained reaction mixture was stirred for 12 hours. After adding purified water (1 L), the mixture was filtered under reduced pressure. The obtained solid was triturated with MeOH/EA, was triturated with DMF, and then was triturated with EA/THF. The resultant was dissolved in MC, was filtered through silica, and then was triturated with MeOH/EA. Compound C-15 (5.1 g, 57%) was obtained.

### Preparation Example 5

### Preparation of Compound C-29

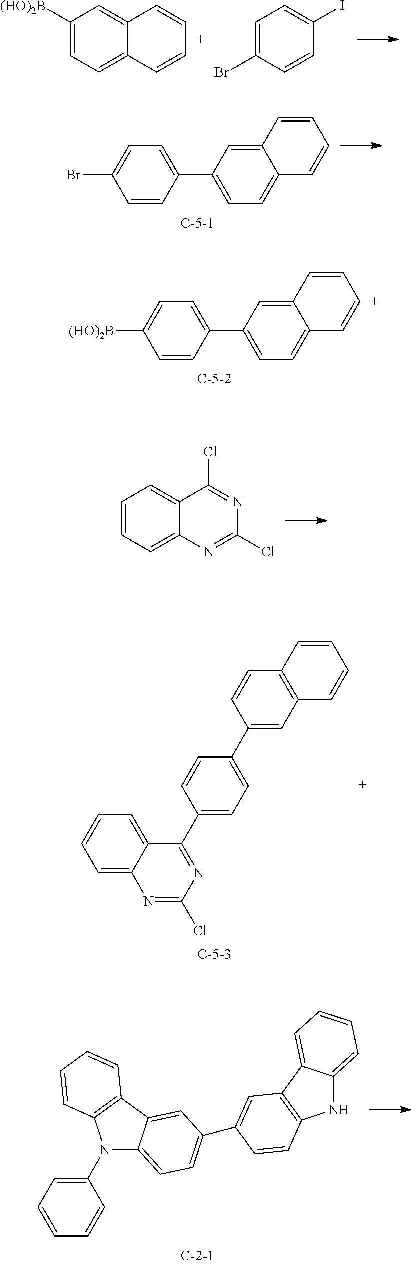

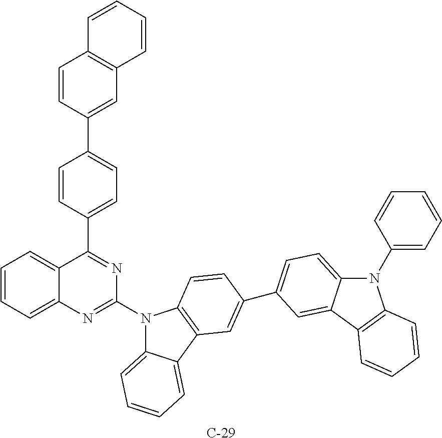

### Preparation of Compound C-5-1

After dissolving 2-naphthylboronic acid (157 g, 554 mmol), 1-bromo-4-iodobenzene (100 g, 581.7 mmol), Pd(PPh₃)₄ (13 g, 11.08 mmol) and Na₂CO₃ (150 g, 1.385 mol) in toluene (3.5 L), EtOH (0.7 L) and distilled water (0.7 L), the reaction mixture was stirred for 3 hours at 90° C. The mixture was extracted with EA and distilled water, was dissolved in chloroform (10 L) by heat, and then was filtered through silica. After triturating the resultant with EA and hexane, the resultant was triturated with EA and MeOH to obtain compound C-5-1 (94 g, 60%).

### Preparation of Compound C-5-2

After dissolving compound C-5-1 (94 g, 332 mmol) in THF (800 mL), 2.5 M n-BuLi in hexane (80 mL, 386.4 mmol) was added to the reaction mixture at −78° C., and then the mixture was stirred for 1 hour. B(OMe)₃ (28 mL, 498 mmol) was added slowly to the mixture, and then the mixture was stirred for 2 hours. The mixture was quenched by adding 2 M HCl, was extracted with distilled water and EA, and then was recrystallized with hexane and acetone. Compound C-5-2 (57 g, 67.0%) was obtained.

### Preparation of Compound C-5-3

2,4-dichloroquinazoline (46 g, 230 mmol), compound C-5-2 (57 g, 230 mmol), Pd(PPh₃)₄ (10.6 g, 9.2 mmol) and Na₂CO₃ (73 g, 690 mmol) were dissolved in toluene (1.1 L), EtOH (230 mL) and distilled water (350 mL), and then was stirred for 5 hours at 100° C. The reaction mixture was distillated under reduced pressure to obtain an organic layer, and then was triturated with MeOH. The obtained solid was dissolved in MC, was filtered through silica, and then was triturated with MC and hexane to obtain compound C-5-3 (51 g, 99.9%).

### Preparation of Compound C-29

After suspending compound C-2-1 (5 g, 12.2 mmol) and compound C-5-3 (4.5 g, 12.2 mmol) in DMF 80 mL, 60% NaH (881 mg, 22 mmol) was added to the mixture at room temperature. The obtained reaction mixture was stirred for 12 hours. After adding purified water (1 L), the mixture was filtered under reduced pressure. The obtained solid was triturated with MeOH/EA, was triturated with DMF, and then was triturated with EA/THF. The resultant was dissolved in MC, was filtered through silica, and then was triturated with MeOH/EA. Compound C-29 (1.8 g, 20%) was obtained.

### Preparation Example 6

### Preparation of Compound C-84

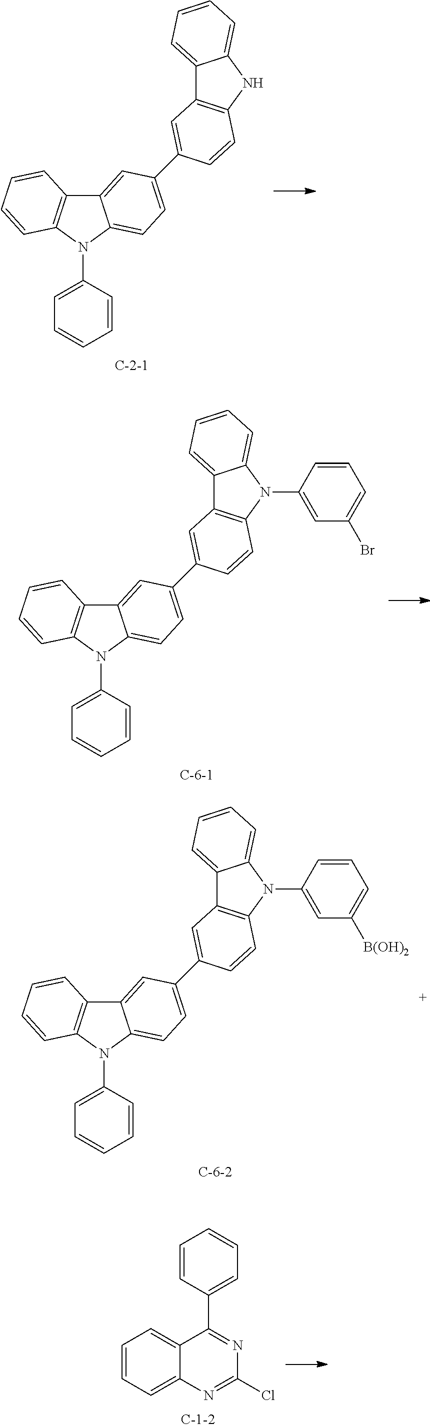 [SMILES: c1ccc(-n2c3ccccc3c3cc(-c4ccc5[nH]c6ccccc6c5c4)ccc32)cc1 ; Brc1cccc(-n2c3ccccc3c3cc(-c4ccc5c(c4)c4ccccc4n5-c4ccccc4)ccc32)c1 ; OB(O)c1cccc(-n2c3ccccc3c3cc(-c4ccc5c(c4)c4ccccc4n5-c4ccccc4)ccc32)c1 ; Clc1nc(-c2ccccc2)c2ccccc2n1]

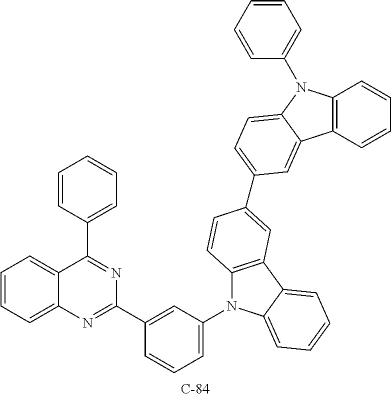

### Preparation of Compound C-6-1

Compound C-2-1 (14 g, 34.3 mmol), 1,3-dibromobenzene (48.5 g, 171.4 mmol), CuI (3.3 g, 17.1 mmol), K₃PO₄ (21.8 g, 102.9 mmol) and EDA (2.3 mL, 34.3 mmol) were added to toluene 500 mL. The reaction mixture was stirred under reflux for 1 day, was extracted with EA, and then was distilled under reduced pressure. After purifying the resultant by column chromatography with MC/Hexane, compound C-6-1 (15.5 g, 80.1%) was obtained.

### Preparation of Compound C-6-2

After dissolving compound C-6-1 (15.5 g, 27.5 mmol) in THF (250 mL), 2.5 M n-BuLi in hexane (17.6 mL, 44 mmol) was added thereto at −78° C. The reaction mixture was stirred for 1 hour. B(Oi-Pr)₃ (12.6 mL, 55 mmol) was added slowly to the mixture, and then the mixture was stirred for 2 hours. The mixture was quenched by adding 2 M HCl, was extracted with distilled water and EA, and then was recrystallized with hexane and MC. Compound C-6-2 (8.7 g, 60%) was obtained.

### Preparation of Compound C-84

After compound C-1-2 (2.3 g, 9.5 mmol), compound C-6-2 (6 g, 11.3 mmol), Pd(PPh₃)₄ (532 mg, 0.46 mmol) and Na₂CO₃ (2.9 g, 27.6 mmol) were dissolved in toluene (55 mL), EtOH (14 mL) and distilled water (14 mL), the reaction mixture was stirred for 2 hours at 90° C. The mixture was extracted with distilled water and EA. After purifying the resultant by a column chromatography with MC and hexane, compound C-84 (2.4 g, 36.9%) was obtained.

### Preparation Example 7

### Preparation of Compound C-86

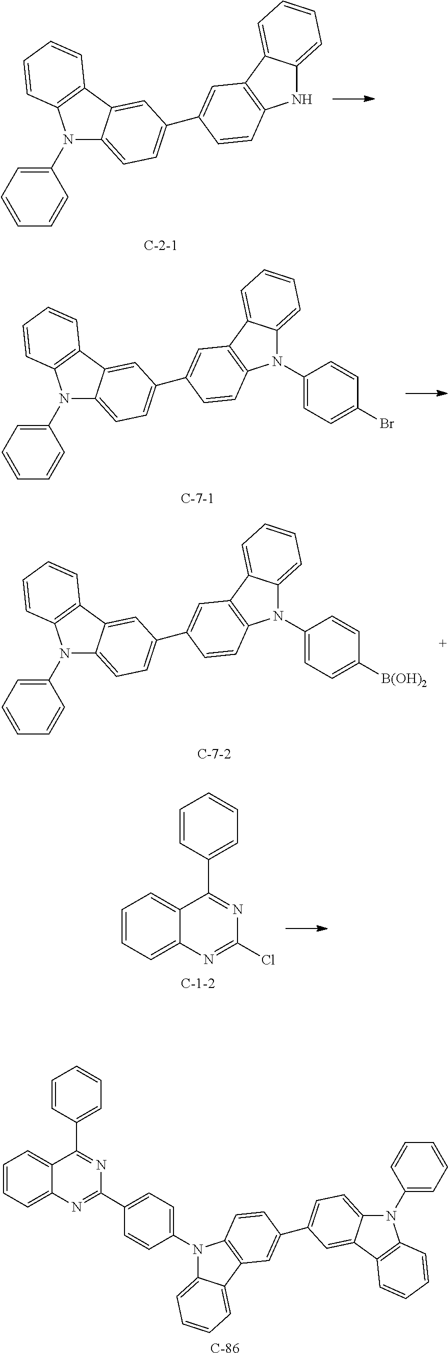

### Preparation of Compound C-7-1

Compound C-2-1 (14 g, 34.3 mmol), 1-bromo-4-iodobenzene (48.5 g, 171.4 mmol), CuI (3.3 g, 17.1 mmol), K₃PO₄ (21.8 g, 102.9 mmol) and EDA (2.3 mL, 34.3 mmol) were added to toluene 500 mL. The reaction mixture was stirred under reflux for 1 day, was extracted with EA, and then was distilled under reduced pressure. After purifying the resultant by column chromatography with MC/Hexane, compound C-7-1 (15.5 g, 80.1%) was obtained.

### Preparation of Compound C-7-2

After dissolving compound C-7-1 (15.5 g, 27.5 mmol) in THF (250 mL), 2.5 M n-BuLi in hexane (17.6 mL, 44 mmol) was added thereto at −78° C. The reaction mixture was stirred for 1 hour. B(Oi-Pr)₃ (12.6 mL, 55 mmol) was added slowly to the mixture, and then the mixture was stirred for 2 hours. The mixture was quenched by adding 2 M HCl, was extracted with distilled water and EA, and then was recrystallized with MC and hexane. Compound C-7-2 (8.7 g, 60%) was obtained.

### Preparation of Compound C-86

After compound C-1-2 (2.3 g, 9.5 mmol), compound C-7-2 (6 g, 11.3 mmol), Pd(PPh₃)₄ (532 mg, 0.46 mmol) and Na₂CO₃ (2.9 g, 27.6 mmol) were dissolved in toluene (55 mL), EtOH (14 mL) and distilled water (14 mL), the reaction mixture was stirred for 2 hours at 90° C. The mixture was extracted with distilled water and EA. After purifying the resultant by a column chromatography with MC and hexane, compound C-86 (2.4 g, 36.9%) was obtained.

### Preparation Example 8

### Preparation of Compound C-87

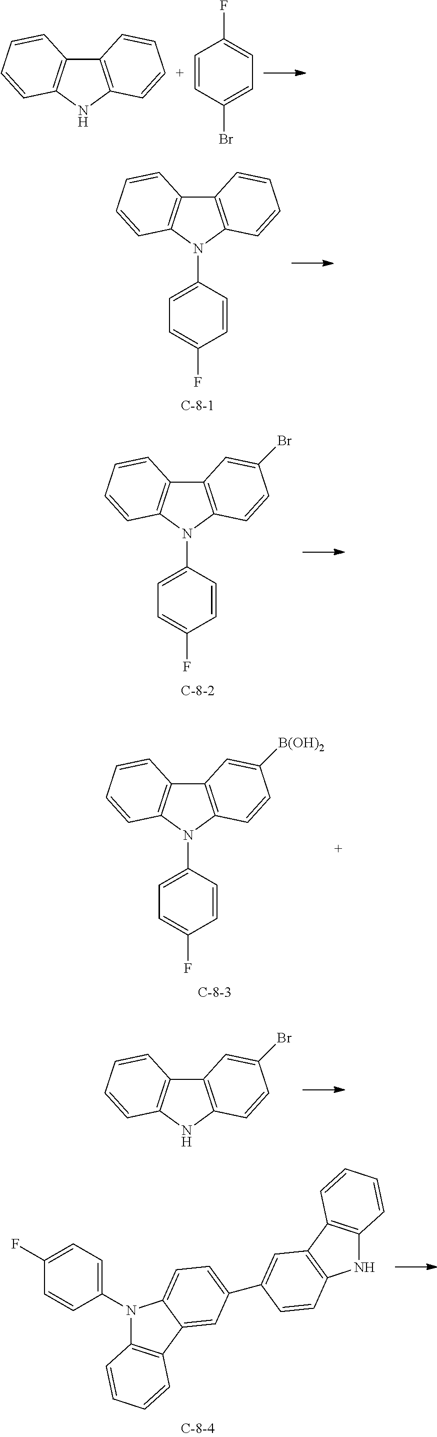

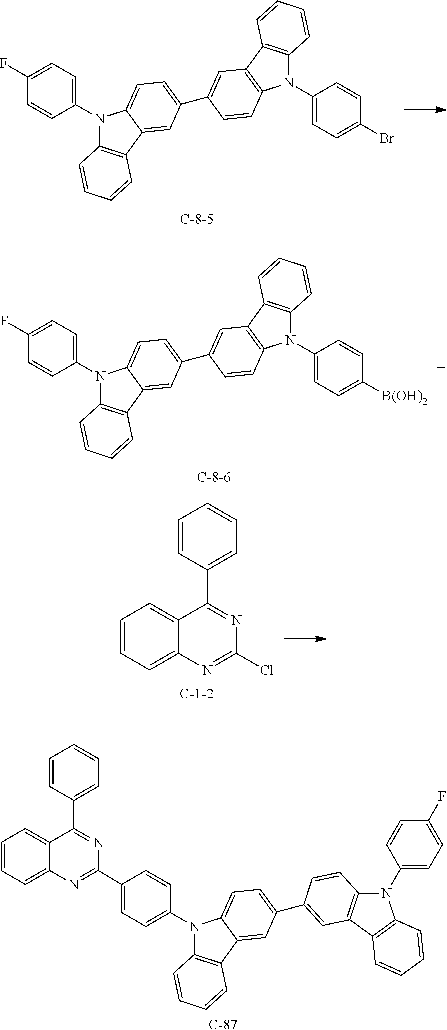

### Preparation of Compound C-8-1

9H-carbazole (20 g, 119.6 mmol), 1-bromo-4-fluorobenzene (40 mL, 358.8 mmol), CuI (23 g, 119.6 mmol), K₃PO₄ (117 g, 357 mmol) and EDA (16 mL, 238 mmol) were added to toluene 500 mL. The reaction mixture was stirred under reflux for 1 day, was extracted with EA, and then was distilled under reduced pressure. After purifying the resultant by column chromatography with MC/Hexane, compound C-8-1 (42 g, 67%) was obtained.

### Preparation of Compound C-8-2

After dissolving compound C-8-1 (5 g, 19.1 mmol) in DMF (100 mL), NBS (3.4 g, 19.1 mmol) was added thereto. The reaction mixture was stirred for 1 day, was extracted with EA, and then was distilled under reduced pressure. After purifying the resultant by column chromatography with MC/Hexane, compound C-8-2 (5.6 g, 86%) was obtained.

### Preparation of Compound C-8-3

After dissolving C-8-2 (5.6 g, 16.5 mmol) in THF (85 mL), 2.5 M n-BuLi in hexane (7.2 mL, 18.2 mmol) was added thereto at −78° C. The reaction mixture was stirred for 1 hour. B(Oi-Pr)₃ (5.7 mL, 24.7 mmol) was added slowly to the mixture, and then the mixture was stirred for 2 hours. The mixture was quenched by adding 2 M HCl, was extracted with distilled water and EA, and then was recrystallized with MC and hexane. Compound C-8-3 (8.7 g, 60%) was obtained.

### Preparation of Compound C-8-4

Compound C-8-3 (14 g, 48.76 mmol), 3-bromo-9H-carbazole (10 g, 40.63 mmol), K₂CO₃ (13.5 g, 97.52 mmol) and Pd(PPh₃)₄ (2.35 g, 2.03 mmol) were added to toluene 200 mL, EtOH 50 mL, and purified water 50 mL. After stirring the reaction mixture for 3 hours at 90 to 100° C., the mixture was cooled to room temperature. An aqueous layer was removed from the mixture by a gravity separation. The obtained organic layer was concentrated, was recrystallized with MC, and then was filtered to obtain compound C-8-4 (12 g, 72%).

### Preparation of Compound C-8-5

Compound C-8-4 (14 g, 34.3 mmol), 1-bromo-4-iodobenzene (48.5 g, 171.4 mmol), CuI (3.3 g, 17.1 mmol), K₃PO₄ (21.8 g, 102.9 mmol) and EDA (2.3 mL, 34.3 mmol) were added to toluene 500 mL. The reaction mixture was stirred under reflux for 1 day, was extracted with EA, and then was distilled under reduced pressure. After purifying the resultant by column chromatography with MC/Hexane, compound C-8-5 (15.5 g, 80.1%) was obtained.

### Preparation of Compound C-8-6

After dissolving compound C-8-5 (15.5 g, 27.5 mmol) in THF (250 mL), 2.5 M n-BuLi in hexane (17.6 mL, 44 mmol) was added to the reaction mixture at −78° C., and then the mixture was stirred for 1 hour. B(Oi-Pr)₃ (12.6 mL, 55 mmol) was added slowly to the mixture, and then the mixture was stirred for 2 hours. The mixture was quenched by adding 2 M HCl, was extracted with distilled water and EA, and then was recrystallized with MC and hexane. Compound C-8-6 (8.7 g, 60%) was obtained.

### Preparation of Compound C-87

After dissolving compound C-1-2 (2.3 g, 9.5 mmol), compound C-8-6 (6 g, 11.3 mmol), Pd(PPh₃)₄ (532 mg, 0.46 mmol) and Na₂CO₃ (2.9 g, 27.6 mmol) in toluene (55 mL), EtOH (14 mL), and distilled water (14 mL), the reaction mixture was stirred for 2 hours at 90° C., and then was extracted with distilled water and EA. After purifying the resultant by a column chromatography with MC and hexane, compound C-87 (2.4 g, 36.9%) was obtained.

### Preparation Example 9

### Preparation of Compound C-99

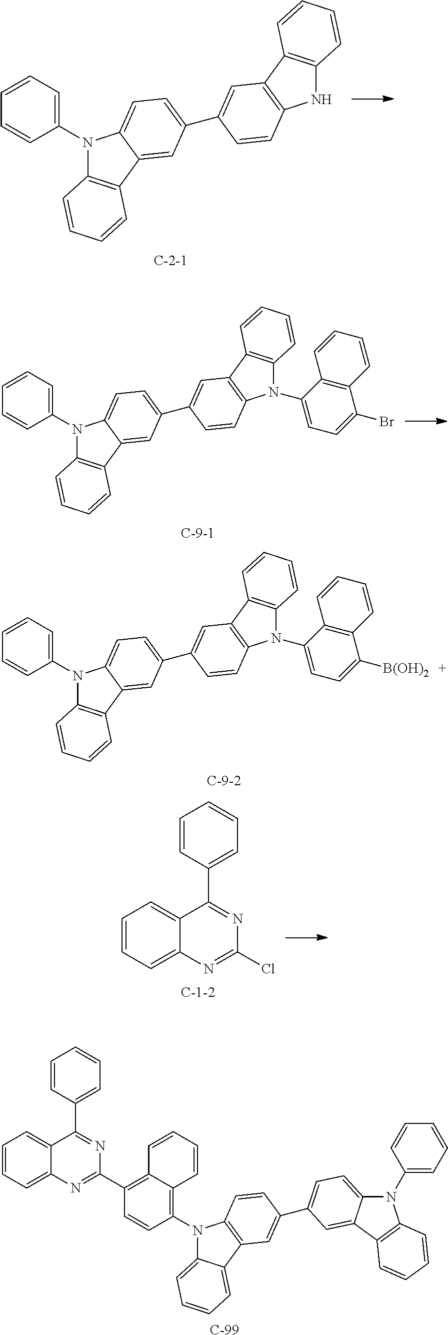

### Preparation of Compound C-9-1

Compound C-2-1 (16 g, 39.17 mmol), 1,4-dibromonaphthalene (28 g, 97.92 mmol), CuI (7.7 g, 40.43 mmol), CsCO₃ (38.4 g, 117.86 mmol) and KI (13 g, 78.3 mmol) were added to toluene 400 mL. After adding ethylenediamine (5.12 mL, 78.3 mmol) thereto, the reaction mixture was stirred under reflux for 30 hours. After completing the reaction, the mixture was cooled to room temperature and was extracted with MC/purified water. The obtained organic layer was concentrated. After purifying the resultant by a silica column chromatography, compound C-9-1 (7.1 g, 30%) was obtained.

### Preparation of Compound C-9-2

After dissolving compound C-9-1 (6 g, 9.78 mmol) in THF (60 mL), 2.5 M n-BuLi in hexane (5.9 mL, 14.7 mmol) was added thereto at −78° C. The reaction mixture was stirred for 1 hour. B(Oi-Pr)₃ (4.5 mL, 19.6 mmol) was added slowly to the mixture, and then the mixture was stirred for 12 hours. After completing the reaction, purified water 20 mL was slowly dropped stepwise to the mixture. Thereafter, the mixture was extracted with MC/NH₄Cl aq. The obtained organic layer was concentrated, and then was filtered through silica to obtain compound C-9-2 (4.5 g, 79.5%).

### Preparation of Compound C-99

After adding compound C-9-2 (4.5 g, 7.78 mmol), compound C-1-2 (2 g, 8.56 mmol), Na₂CO₃ (2.5 g, 23.34 mmol) and Pd(PPh₃)₄ (0.45 g, 0.39 mmol) to toluene 40 mL, EtOH 10 mL and purified water 10 mL, the reaction mixture was stirred for 12 hours at 115 to 120° C. After completing the reaction, the mixture was cooled to room temperature. An aqueous layer was removed from the mixture by a gravity separation. After purifying the obtained organic layer by a silica column chromatography, compound C-99 (3 g, 52.6%) was obtained.

### Preparation Example 10

### Preparation of Compound C-106

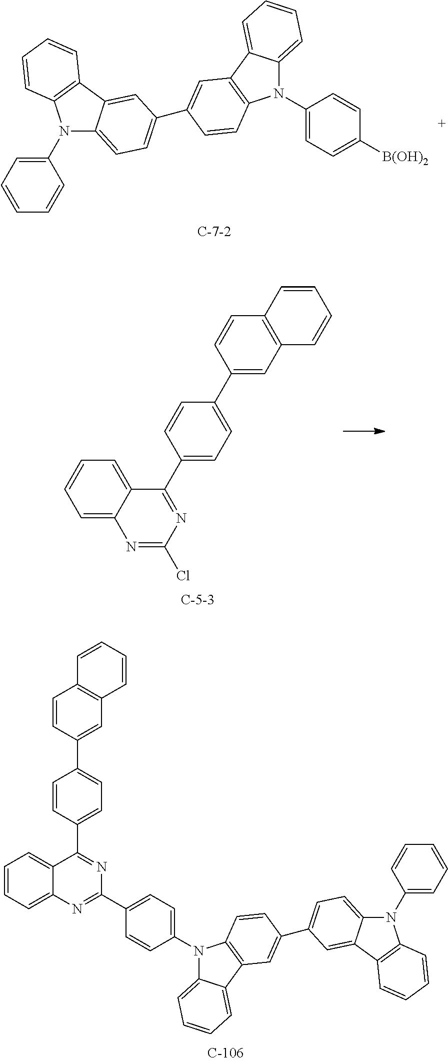 [SMILES: OB(O)c1ccc(-n2c3ccccc3c3cc(-c4ccc5c(c4)c4ccccc4n5-c4ccccc4)ccc32)cc1 ; Clc1nc(-c2ccc(-c3ccc4ccccc4c3)cc2)c2ccccc2n1 ; c1ccc(-n2c3ccccc3c3cc(-c4ccc5c(c4)c4ccccc4n5-c4ccc(-c5nc(-c6ccc(-c7ccc8ccccc8c7)cc6)c6ccccc6n5)cc4)ccc32)cc1]

### Preparation of Compound C-106

After dissolving compound C-7-2 (2.5 g, 4.73 mmol), compound C-5-3 (1.7 g, 4.73 mmol), Pd(PPh₃)₄ (273 mg, 0.24 mmol) and Na₂CO₃ (1.5 g, 14.2 mmol) in toluene (55 mL), EtOH (14 mL) and distilled water (14 mL), the reaction mixture was stirred for 2 hours at 90° C., and then was extracted with distilled water and EA. After purifying the resultant by a column chromatography with MC and hexane, compound C-106 (2.3 g, 59.7%) was obtained.

### Preparation Example 11

### Preparation of Compound C-109

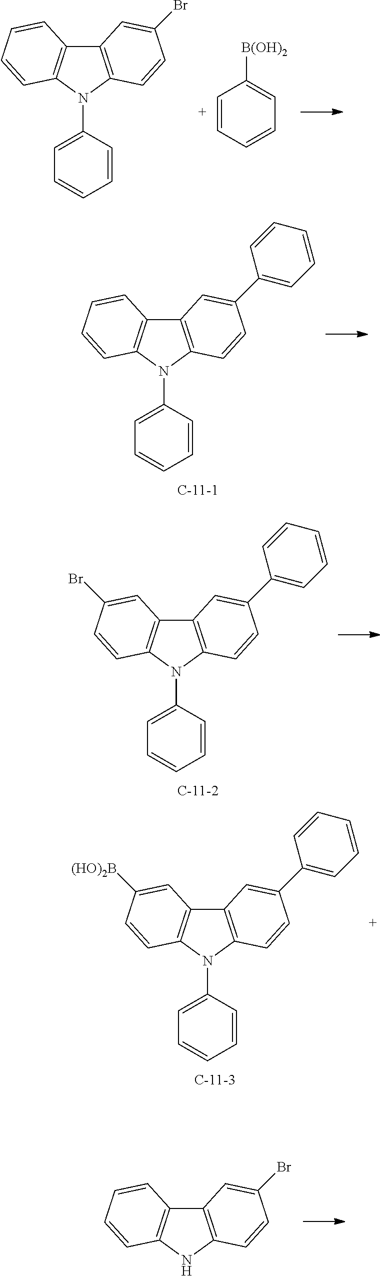 [SMILES: Brc1ccc2c(c1)c1ccccc1n2-c1ccccc1 ; OB(O)c1ccccc1 ; c1ccc(-c2ccc3c(c2)c2ccccc2n3-c2ccccc2)cc1 ; Brc1ccc2c(c1)c1cc(-c3ccccc3)ccc1n2-c1ccccc1 ; OB(O)c1ccc2c(c1)c1cc(-c3ccccc3)ccc1n2-c1ccccc1 ; Brc1ccc2[nH]c3ccccc3c2c1]

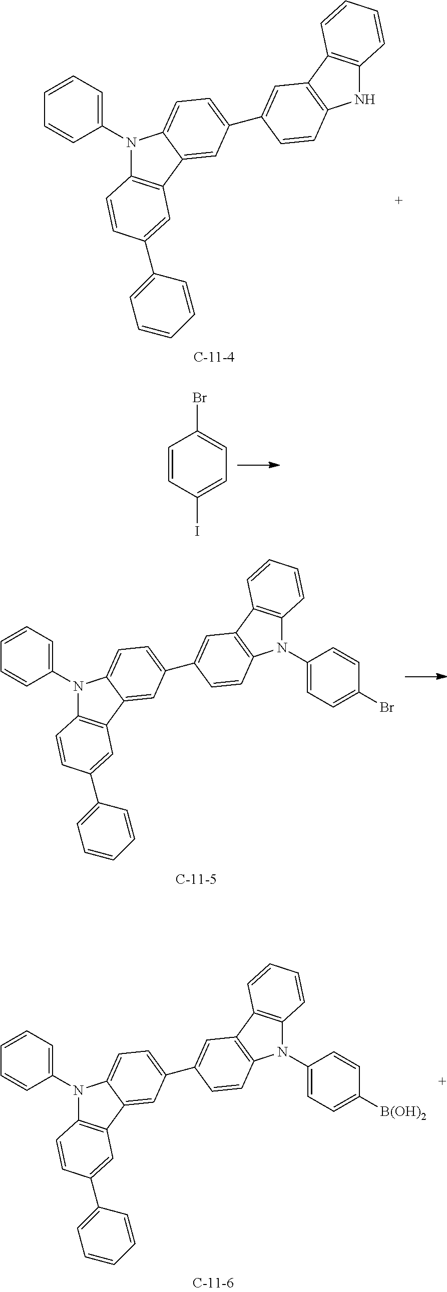 [SMILES: c1ccc(-c2ccc3c(c2)c2cc(-c4ccc5[nH]c6ccccc6c5c4)ccc2n3-c2ccccc2)cc1 ; Brc1ccc(I)cc1 ; Brc1ccc(-n2c3ccccc3c3cc(-c4ccc5c(c4)c4cc(-c6ccccc6)ccc4n5-c4ccccc4)ccc32)cc1 ; OB(O)c1ccc(-n2c3ccccc3c3cc(-c4ccc5c(c4)c4cc(-c6ccccc6)ccc4n5-c4ccccc4)ccc32)cc1]

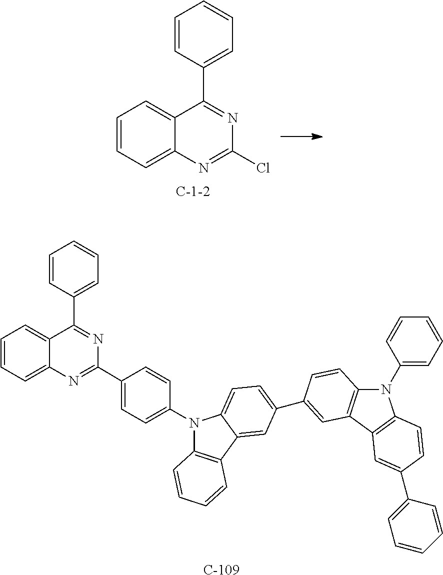 [SMILES: Clc1nc(-c2ccccc2)c2ccccc2n1 ; c1ccc(-c2ccc3c(c2)c2cc(-c4ccc5c(c4)c4ccccc4n5-c4ccc(-c5nc(-c6ccccc6)c6ccccc6n5)cc4)ccc2n3-c2ccccc2)cc1 ; "C-109"]

### Preparation of Compound C-11-1

After dissolving 3-bromo-9-phenyl-9H-carbazole (10 g, 31.06 mmol), phenylboronic acid (3.75 g, 31.06 mmol), K₂CO₃ (12.9 g, 93.18 mmol) and Pd(PPh₃)₄ (1.8 g, 1.55 mmol) in toluene 150 mL, EtOH 40 mL and purified water 40 mL, the reaction mixture was stirred for 3 hours at 90 to 100° C. After completing the reaction, the mixture was cooled to room temperature. An aqueous layer was removed from the mixture by a gravity separation. After purifying the obtained organic layer by a silica column chromatography, compound C-11-1 (6.4 g, 65%) was obtained.

### Preparation of Compound C-11-2

After dissolving compound C-11-1 (6.4 g, 20.06 mmol) in DMF 100 mL, NBS (3.6 g, 20.06 mmol) was added thereto. The reaction mixture was stirred for 3 hours. After completing the reaction, the mixture was extracted with MC/purified water. After purifying the resultant by a silica column chromatography, compound C-11-2 (4.8 g, 60%) was obtained.

### Preparation of Compound C-11-3

After dissolving compound C-11-2 (4.8 g, 12.06 mmol) in THF (60 mL), 2.5 M n-BuLi in hexane (6.3 mL, 15.68 mmol) was added thereto at −78° C. The reaction mixture was stirred for 1 hour. B(Oi-Pr)₃ (4.5 g, 24.12 mmol) was added slowly to the mixture, and then the mixture was stirred for 12 hours. After completing the reaction, purified water 20 mL was slowly dropped stepwise to the mixture. Thereafter, the mixture was extracted with MC/NH₄Cl aq. The obtained organic layer was concentrated, was filtered through silica, and then was crystallized with MC/hexane to obtain compound C-11-3 (3 g, 70%).

### Preparation of Compound C-11-4

3-bromo-9H-carbazole (2 g, 8.26 mmol), compound C-11-3 (3 g, 8.26 mmol), Pd(PPh₃)₄ (0.48 g, 0.4 mmol) and K₂CO₃ (3.4 g, 24.78 mmol) were added to toluene 40 mL, EtOH 10 mL and purified water 10 mL. The reaction mixture was stirred for 15 hours at 70 to 80° C. After completing the reaction, an aqueous layer was removed from the mixture by a gravity separation. The obtained organic layer was concentrated. After purifying the resultant by a silica column chromatography, compound C-11-4 (3.2 g, 80%) was obtained.

### Preparation of Compound C-11-5

After adding compound C-11-4 (3.2 g, 6.6 mmol), iodobromobenzene (3.7 g, 13.21 mmol), CuI (1.5 g, 7.9 mmol) and K₃PO₄ (2.8 g, 13.2 mmol) to toluene 33 mL, ethylenediamine (0.47 g, 7.9 mmol) was added thereto. The reaction mixture was stirred under reflux for 30 hours. After completing the reaction, the mixture was cooled to room temperature, and then was extracted with MC/purified water. The obtained organic layer was concentrated. After purifying the resultant by a silica column chromatography, compound C-11-5 (3.3 g, 80%) was obtained.

### Preparation of Compound C-11-6

After dissolving compound C-11-5 (3.3 g, 5.16 mmol) in THF (25 mL), 2.5 M n-BuLi in hexane (2.6 mL, 6.7 mmol) was added thereto at −78° C. The reaction mixture was stirred for 1 hour. B(Oi-Pr)₃ (1.9 g, 10.3 mmol) was added slowly to the mixture, and then the mixture was stirred for 12 hours. After completing the reaction, purified water 10 mL was slowly dropped stepwise to the mixture. Thereafter, the mixture was extracted with MC/NH₄Cl aq. The obtained organic layer was concentrated, was filtered through silica, and then was recrystallized with MC/hexane to obtain compound C-11-6 (2.5 g, 80%).

### Preparation of Compound C-109

After adding C-11-6 (2.5 g, 4.14 mmol), compound C-1-2 (1 g, 4.55 mmol), Na₂CO₃ (1.3 g, 12.42 mmol) and Pd(PPh₃)₄ (0.24 g, 0.2 mmol) to toluene 20 mL, EtOH 5 mL and purified water 5 mL, the reaction mixture was stirred for 12 hours at 115 to 120° C. After completing the reaction, the mixture was cooled to room temperature. An aqueous layer was removed from the mixture by a gravity separation. After purifying the obtained organic layer by a silica column chromatography, compound C-109 (2.2 g, 70%) was obtained.

Compounds C-1, C-5, C-6, C-10, C-11, C-18, C-52, C-68, C-95, C-103 and C-120 to C-125 were prepared by employing the methods of preparation examples 1 to 11. Physicochemical properties of all the prepared compounds are shown in the following Table 1.

TABLE 1

| | Yield | UV | PL | mp | MS/EIMS | |
|---|---|---|---|---|---|---|
| Compound | (%) | (nm) | (nm) | (° C.) | Found | Calculated |
| C-1 | 62 | 306 | 516 | 219 | 613 | 612.72 |
| C-3 | 51.5 | | | | 538 | 537.61 |
| C-5 | 48 | 324 | 525 | 234 | 663 | 662.78 |
| C-6 | 47 | 356 | 513 | 245 | 663 | 662.78 |
| C-9 | 47.4 | 342 | 523 | 265 | 688 | 688.26 |
| C-10 | 53 | 304 | 517 | 204 | 689 | 688.82 |
| C-11 | 44 | 308 | 511 | 248 | 729 | 728.88 |
| C-12 | 52 | | | | 630 | 629.77 |
| C-15 | 57 | 304 | 517 | 227 | 764 | 764.29 |
| C-18 | 51 | 354 | 527 | 310 | 765 | 764.91 |
| C-29 | 20 | 342 | 531 | 262 | 738 | 738.28 |
| C-52 | 61 | 310 | 522 | 221 | 765 | 764.91 |
| C-68 | 59 | 304 | 427 | 131 | 536 | 535.64 |
| C-84 | 36.9 | 304 | 383 | 168 | 688 | 688.26 |
| C-86 | 36.9 | 304 | 446 | 168 | 688 | 688.26 |
| C-87 | 36.9 | | | | 706 | 706.26 |
| C-95 | 50 | 344 | 460 | 205 | 765 | 764.91 |
| C-99 | 52.6 | 305 | 464 | 210 | 738 | 738.28 |
| C-103 | 43 | 304 | 443 | 197 | 707 | 706.81 |
| C-106 | 59.7 | | | | 814 | 814.31 |
| C-109 | 70 | 305 | 448 | 187 | 764 | 764.29 |
| C-120 | 45 | 306 | 467 | 210 | 593 | 592.73 |
| C-121 | 47 | 308 | 515 | 235 | 627 | 626.75 |
| C-122 | 53 | 338 | 505 | 274 | 669 | 668.83 |
| C-123 | 56 | 304 | 427 | 131 | 537 | 536.62 |
| C-124 | 51 | 340 | 513 | 281 | 702 | 701.81 |
| C-125 | 50 | 306 | 508 | 196 | 627 | 626.75 |

### Example 1

### Production of an OLED Device Using the Organic Electroluminescent Compound According to the Present Invention

A transparent electrode indium tin oxide (ITO) thin film (15 Ω/sq) on a glass substrate for an organic light-emitting diode (OLED) device (Samsung Corning, Republic of Korea) was subjected to an ultrasonic washing with trichloroethylene, acetone, ethanol and distilled water, sequentially, and then was stored in isopropanol. Then, the ITO substrate was mounted on a substrate holder of a vacuum vapor depositing apparatus. N¹-(naphthalen-1-yl)-N⁴,N⁴-diphenylbenzene-1,4-diamine was introduced into a cell of said vacuum vapor depositing apparatus, and then the pressure in the chamber of said apparatus was controlled to achieve 10⁻⁶ torr. Thereafter, an electric current was applied to the cell to evaporate the above introduced material, thereby forming a hole injection layer having a thickness of 60 nm on the ITO substrate. Then, N,N′-di(4-biphenyl)-N,N′-di(4-biphenyl)-4,4′-diaminobiphenyl was introduced into another cell of said vacuum vapor depositing apparatus, and was evaporated by applying electric current to the cell, thereby forming a hole transport layer having a thickness of 20 nm on the hole injection layer. Thereafter, compound C-1 was introduced into one cell of the vacuum vapor depositing apparatus, as a host material, and compound D-7 was introduced into another cell as a dopant. The two materials were evaporated at different rates and was deposited in a doping amount of 4 to 20 wt % to form a light-emitting layer having a thickness of 30 nm on the hole transport layer. Then, 9,10-di(1-naphthyl)-2-(4-phenyl-1-phenyl-1H-benzo[d]imidazole)anthracene was introduced into one cell and lithium quinolate was introduced into another cell. The two materials were evaporated at different rates and was deposited in a doping amount of 30 to 70 wt % to form an electron transport layer having a thickness of 30 nm on the light-emitting layer. Then, after depositing lithium quinolate as an electron injection layer having a thickness of 1 to 2 nm on the electron transport layer, an Al cathode having a thickness of 150 nm was deposited by another vacuum vapor deposition apparatus on the electron injection layer. Thus, an OLED device was produced. All the material used for producing the OLED device were those purified by vacuum sublimation at 10⁻⁶ torr.

The produced OLED device shows red emission having a luminance of 1,020 cd/m² at a driving voltage of 4.3 V and a current density of 7.5 mA/cm². Further, the minimum time for a luminance of 5,000 nit to be reduced to 90% of the luminance was 140 hours.

### Examples 2 to 11

### Production of an OLED Device Using the Organic Electroluminescent Compound According to the Present Invention

OLED devices were produced in the same manner as one of Example 1, except for using those shown in the below Table 2 as a host material and a dopant.

### Comparative Example 1

### Production of an OLED Device Using Conventional Electroluminescent Compounds

OLED device was produced in the same manner as one of Example 1, except that a light-emitting layer having a thickness of 30 nm was deposited on the hole transport layer by using 4,4′-N,N′-dicarbazol-biphenyl (CBP) as a host material and (piq)₂Ir(acac) [bis-(1-phenylisoquinolyl)iridium(III)acetylacetonate] as a dopant and that a hole blocking layer having a thickness of 10 nm was deposited by using aluminum(III)bis(2-methyl-8-quinolinato)-4-phenylphenolate.

The produced OLED device shows red emission having a luminance of 1,000 cd/m² at a driving voltage of 5.5 V and a current density of 12.5 mA/cm². Further, minimum time for a luminance of 5,000 nit to be reduced to 90% of the luminance was 15 hours.

The results of examples and comparative example are shown in the following Table 2.

TABLE 2

| | | | | | | Minimum Time |
|---|---|---|---|---|---|---|
| | | | | | | Required for a |
| | | | | | | Luminance of |
| | | | | Current | | 5,0005,000 |
| | Host | | Driving | Density | Luminance | nit to Be |
| | Material | Dopant | Voltage (V) | (mA/cm²) | (cd/m²)/Color | Reduced to 90% |
| Example 1 | C-1 | D-7 | 4.3 | 7.5 | 1,020/Red | 140 |
| Example 2 | C-4 | D-11 | 4.0 | 10.2 | 1,030/Red | 70 |
| Example 3 | C-9 | D-7 | 4.4 | 7.8 | 1,050/Red | 140 |
| Example 4 | C-13 | D-7 | 4.3 | 8.3 | 1,100/Red | 150 |
| Example 5 | C-28 | D-11 | 4.2 | 9.8 | 1,030/Red | 70 |
| Example 6 | C-52 | D-11 | 4.4 | 10.2 | 1,070/Red | 80 |
| Example 7 | C-67 | D-7 | 4.5 | 7.1 | 1,010/Red | 140 |
| Example 8 | C-99 | D-11 | 4.2 | 10.6 | 1,080/Red | 80 |
| Example 9 | C-11 | D-11 | 4.0 | 10.2 | 1,050/Red | 100 |
| Example 10 | C-86 | D-11 | 4.1 | 9.8 | 1,070/Red | 100 |
| Example 11 | C-121 | D-7 | 4.2 | 7.2 | 1,080/Red | 120 |
| Comparative | CBP | (piq)₂Ir | 5.5 | 12.5 | 1,000/Red | 15 |
| Example 1 | | (acac) | | | | |

As shown in Table 2, the organic electroluminescent compounds according to the present invention have superior properties than those of conventional electroluminescent compounds, and thus provide an organic electroluminescent device which has high luminous efficiency and a long operation lifetime and requires a low driving voltage improving power efficiency and power consumption.

1. A compound represented by the following formula 1: 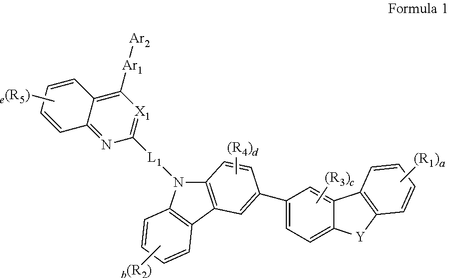
wherein
L₁ represents a single bond or a substituted or unsubstituted (C6-C30)arylene group;
X₁ represents N;
Y represents —NR₁₃—;
Ar₁ represents a single bond, a substituted or unsubstituted 5- to 30-membered heteroarylene group, a substituted or unsubstituted (C6-C30)arylene group, or a substituted or unsubstituted (C1-C30)alkylene group;
Ar₂ represents hydrogen, deuterium, a substituted or unsubstituted (C1-C30)alkyl group, a substituted or unsubstituted (C6-C30)aryl group, or a substituted or unsubstituted 5- to 30-membered heteroaryl group;
R₁ to R₅ each independently represent hydrogen, deuterium, a halogen, a substituted or unsubstituted (C1-C30)alkyl group, a substituted or unsubstituted (C6-C30)aryl group, a substituted or unsubstituted 5- to 30-membered heteroaryl group, a substituted or unsubstituted (C3-C30)cycloalkyl group, a substituted or unsubstituted 5- to 7-membered heterocycloalkyl group, a substituted or unsubstituted (C6-C30)aryl(C1-C30)alkyl group, a substituted or unsubstituted (C6-C30)aryl group fused with at least one (C3-C30)cycloalkyl group, a 5- or 7-membered heterocycloalkyl group fused with at least one substituted or unsubstituted (C6-C30)aromatic ring, (C3-C30)cycloalkyl group fused with at least one substituted or unsubstituted (C6-C30)aromatic ring, —SiR₁₆R₁₇R₁₈, a cyano group; or are linked to an adjacent substituent via a substituted or unsubstituted (C3-C30)alkylene group or a substituted or unsubstituted (C3-C30)alkenylene group to form a mono- or polycyclic alicyclic ring or a mono- or polycyclic aromatic ring whose carbon atom(s) may be substituted by at least one hetero atom selected from nitrogen, oxygen and sulfur;
R₁₃ represents a substituted or unsubstituted (C6-C30)aryl group, a substituted or unsubstituted (C6-C30)aryl(C1-C30)alkyl group, or a substituted or unsubstituted (C6-C30)aryl group fused with at least one (C3-C30)cycloalkyl group;
R₁₆ to R₁₈ have the same meaning as one of R₁ to R₅;
a, b and e each independently represent an integer of 1 to 4; where a, b or e is an integer of 2 or more, each of R₁, each of R₂ or each of R₅ is the same or different; and
c and d each independently represent an integer of 1 to 3; where c or d is an integer of 2 or more, each of R₃ or each of R₄ is the same or different.

2. The compound of claim 1, wherein
Ar₁ represents a single bond or a substituted or unsubstituted (C6-C30)arylene group;
Ar₂ represents hydrogen, deuterium, or a substituted or unsubstituted (C6-C30)aryl group; and
R₁ to R₅ each independently represent hydrogen or deuterium.

3. The compound of claim 1, wherein
Ar₁ represents a single bond or a substituted or unsubstituted (C6-C30)arylene group;
Ar₂ represents hydrogen, deuterium, or a substituted or unsubstituted (C6-C30)aryl group;
R₁ to R₅ each independently represent hydrogen or deuterium; and
L₁ is a single bond.

4. The compound of claim 1, wherein
Ar₁ represents a single bond or a substituted or unsubstituted (C6-C30)arylene group;
Ar₂ represents hydrogen, deuterium, or a substituted or unsubstituted (C6-C30)aryl group;
R₁ to R₅ each independently represent hydrogen or deuterium; and
L₁ represents a substituted or unsubstituted (C6-C30)arylene group.

5. The compound of claim 4 wherein L₁ represents an unsubstituted (C6-C30)arylene group.

6. The compound of claim 1 wherein R₁ represents hydrogen, deuterium, a halogen, an unsubstituted (C1-C30)alkyl group, an unsubstituted (C6-C30)aryl group, a substituted or unsubstituted 5- to 30-membered heteroaryl group, an unsubstituted (C3-C30)cycloalkyl group, an unsubstituted 5- to 7-membered heterocycloalkyl group, an unsubstituted (C6-C30)aryl(C1-C30)alkyl group, an unsubstituted (C6-C30)aryl group fused with at least one (C3-C30)cycloalkyl group, a 5- or 7-membered heterocycloalkyl group fused with at least one unsubstituted (C6-C30)aromatic ring, (C3-C30)cycloalkyl group fused with at least one unsubstituted (C6-C30)aromatic ring, —SiR₁₆R₁₇R₁₈, a cyano group, or are linked to an adjacent substituent via a substituted or unsubstituted (C3-C30)alkylene group or a substituted or unsubstituted (C3-C30)alkenylene group to form a mono- or polycyclic alicyclic ring or a mono- or polycyclic aromatic ring whose carbon atom(s) may be substituted by at least one hetero atom selected from nitrogen, oxygen and sulfur.

7. The compound of claim 1 wherein R₅ represents hydrogen, deuterium, a halogen, a substituted or unsubstituted (C1-C30)alkyl group, a substituted or unsubstituted (C6-C30)aryl group, a substituted or unsubstituted 5- to 30-membered heteroaryl group, a substituted or unsubstituted (C3-C30)cycloalkyl group, a substituted or unsubstituted 5- to 7-membered heterocycloalkyl group, a substituted or unsubstituted (C6-C30)aryl(C1-C30)alkyl group, a substituted or unsubstituted (C6-C30)aryl group fused with at least one (C3-C30)cycloalkyl group, —SiR₁₆R₁₇R₁₈, or a cyano group.

8. The compound of claim 7 wherein R₅ represents hydrogen or deuterium.

9. The compound of claim 1 wherein R₁ and R₂ each independently represent hydrogen, a substituted or unsubstituted (C6-C30)aryl group, or a substituted or unsubstituted 5- or 30-membered heteroaryl group.

10. The compound of claim 9 wherein R₁ and R₂ each independently represent hydrogen or a substituted or unsubstituted (C6-C30)aryl group.

11. The compound of claim 1 wherein R₃ to R₅ each independently represent hydrogen or a substituted or unsubstituted (C1-C30)alkyl group.

12. The compound of claim 11 wherein R₃ to R₅ each independently represent hydrogen.

13. The compound of claim 1 wherein R₁₃ represents a substituted or unsubstituted (C6-C30)aryl group.

14. An organic electroluminescence device material comprising the compound of claim 1.

15. An organic electroluminescence device comprising: a first electrode, a second electrode, and a plurality of organic layers provided between the first electrode and the second electrode, the organic layers comprising a light-emitting layer, wherein at least one of the organic layers comprises the organic electroluminescence device material of claim 14.

16. The organic electroluminescence device of claim 15, wherein the light-emitting layer comprises the organic electroluminescence device material as a host material.

17. The organic electroluminescence device of claim 15, wherein the light-emitting layer comprises a phosphorescent material.

18. The organic electroluminescence device of claim 17, wherein the phosphorescent material is an ortho-metalated complex of a metal atom selected from iridium (Ir), osmium (Os) and platinum (Pt).

19. The organic electroluminescence device of claim 15, wherein an electron injection layer is provided between an electrode and the light-emitting layer.

20. The organic electroluminescence device of claim 19, wherein the electron injection layer comprises lithium quinolate.

21. The organic electroluminescence device of claim 15, wherein an electron transport layer is provided between an electrode and the light-emitting layer, the electron transport layer comprising the organic electroluminescence device material.

22. The organic electroluminescence device of claim 15, wherein a reductive dopant layer is present between an electrode and at least one of the organic layers.

23. An organic electroluminescent device comprising a first electrode, a second electrode and a plurality of organic layers provided between the first electrode and the second electrode, the organic layers comprising a light-emitting layer, wherein
at least one of the organic layers is the light-emitting layer comprising a host material and a phosphorescent material providing phosphorescence,
the host material being a compound represented by a formula 1 below: 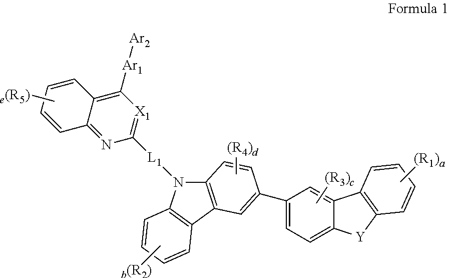
wherein
L₁ represents a single bond or a substituted or unsubstituted (C6-C30)arylene group;
X₁ represents N;
Y represents —NR₁₃—;
Ar₁ represents a single bond, a substituted or unsubstituted 5- to 30-membered heteroarylene group, a substituted or unsubstituted (C6-C30)arylene group, or a substituted or unsubstituted (C1-C30)alkylene group;
Ar₂ represents hydrogen, deuterium, a substituted or unsubstituted (C1-C30)alkyl group, a substituted or unsubstituted (C6-C30)aryl group, or a substituted or unsubstituted 5- to 30-membered heteroaryl group;
R₁ to R₅ each independently represent hydrogen, deuterium, a halogen, a substituted or unsubstituted (C1-C30)alkyl group, a substituted or unsubstituted (C6-C30)aryl group, a substituted or unsubstituted 5- to 30-membered heteroaryl group, a substituted or unsubstituted (C3-C30)cycloalkyl group, a substituted or unsubstituted 5- to 7-membered heterocycloalkyl group, a substituted or unsubstituted (C6-C30)aryl(C1-C30)alkyl group, a substituted or unsubstituted (C6-C30)aryl group fused with at least one (C3-C30)cycloalkyl group, a 5- or 7-membered heterocycloalkyl group fused with at least one substituted or unsubstituted (C6-C30)aromatic ring, (C3-C30)cycloalkyl group fused with at least one substituted or unsubstituted (C6-C30)aromatic ring, —SiR₁₆R₁₇R₁₈, a cyano group; or are linked to an adjacent substituent via a substituted or unsubstituted (C3-C30)alkylene group or a substituted or unsubstituted (C3-C30)alkenylene group to form a mono- or polycyclic alicyclic ring or a mono- or polycyclic aromatic ring whose carbon atom(s) may be substituted by at least one hetero atom selected from nitrogen, oxygen and sulfur;
R₁₃ represents a substituted or unsubstituted (C6-C30)aryl group, a substituted or unsubstituted (C6-C30)aryl(C1-C30)alkyl group, or a substituted or unsubstituted (C6-C30)aryl group fused with at least one (C3-C30)cycloalkyl group;
R₁₆ to R₁₈ have the same meaning as one of R₁ to R₅;
a, b and e each independently represent an integer of 1 to 4; where a, b or e is an integer of 2 or more, each of R₁, each of R₂ or each of R₅ is the same or different; and
c and d each independently represent an integer of 1 to 3; where c or d is an integer of 2 or more, each of R₃ or each of R₄ is the same or different.

24. The organic electroluminescent device of claim 23, wherein the light-emitting layer comprises a host material and phosphorescent material, the phosphorescent material being an ortho-metalated complex of a metal atom selected from iridium (Ir), osmium (Os) and platinum (Pt).

25. The organic electroluminescence device of claim 23, wherein an electron injection layer is provided between an electrode and the light-emitting layer.

26. The organic electroluminescence device of claim 25, wherein the electron injection layer comprises lithium quinolate.

27. The organic electroluminescence device of claim 23, wherein an electron transport layer is provided between an electrode and the light-emitting layer, the electron transport layer comprising a compound represented by the formula 1.

28. The organic electroluminescence device of claim 23, wherein a reductive dopant layer is present between an electrode and at least one of the organic layers.

29. An organic electroluminescent device comprising a first electrode, a second electrode and a plurality of organic layers provided between the first electrode and the second electrode, the organic layers comprising a light-emitting layer, wherein
the light-emitting layer comprises the compound according to claim 1 and a phosphorescent material, wherein the compound is represented by formula 1,
Ar₁ represents a single bond or a substituted or unsubstituted (C6-C30)arylene group,
Ar₂ represents hydrogen, deuterium, or a substituted or unsubstituted (C6-C30)aryl group,
R₁ to R₅ each independently represent hydrogen or deuterium; and
the phosphorescent material is an Ir complex.

30. The organic electroluminescence device of claim 29, wherein L₁ is a single bond.

31. The organic electroluminescence device of claim 29, wherein L₁ represents a substituted or unsubstituted (C6-C30)arylene group.

32. The organic electroluminescence device of claim 31, wherein L₁ represents an unsubstituted (C6-C30)arylene group.

33. A biscarbazole derivative represented by a formula 2 below, 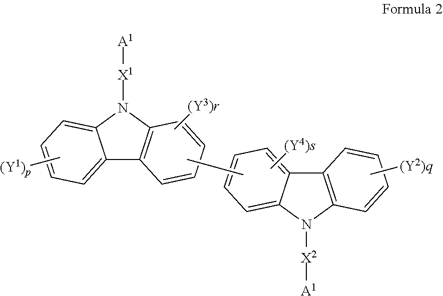
where: A¹ represents a substituted or unsubstituted quinazoline ring bound to X¹ through the nitrogen-containing ring of the quinazoline, when A¹ has a substituent, the substituent of A¹ is an alkyl group having 1 to 20 carbon atoms, alkoxy group having 1 to 20 carbon atoms, haloalkyl group having 1 to 20 carbon atoms, alkylsilyl group having 1 to 10 carbon atoms, arylsilyl group having 6 to 30 ring carbon atoms, cyano group, halogen atom, aromatic hydrocarbon group having 6 to 30 ring carbon atoms, substituted or unsubstituted fused aromatic hydrocarbon group having 10 to 30 ring carbon atoms, or monocyclic aromatic heterocyclic group having 2 to 30 ring carbon atoms;
A² represents a substituted or unsubstituted aromatic hydrocarbon group having 6 to 30 ring carbon atoms, when A² has a substituent, the substituent of A² is an alkyl group having 1 to 20 carbon atoms, alkoxy group having 1 to 20 carbon atoms, haloalkyl group having 1 to 20 carbon atoms, alkylsilyl group having 1 to 10 carbon atoms, arylsilyl group having 6 to 30 ring carbon atoms, cyano group, halogen atom, aromatic hydrocarbon group having 6 to 30 ring carbon atoms, substituted or an unsubstituted fused aromatic hydrocarbon group having 10 to 30 ring carbon atoms, or monocyclic aromatic heterocyclic group having 2 to 30 ring carbon atoms;
X¹ and X² each are a linking group and independently represent a single bond, substituted or unsubstituted aromatic hydrocarbon group having 6 to 30 ring carbon atoms, substituted or unsubstituted fused aromatic hydrocarbon group having 10 to 30 ring carbon atoms, when X¹ and X² each have a substituent, the substituent of X¹ and X² is an alkyl group having 1 to 20 carbon atoms, alkoxy group having 1 to 20 carbon atoms, haloalkyl group having 1 to 20 carbon atoms, alkylsilyl group having 1 to 10 carbon atoms, arylsilyl group having 6 to 30 ring carbon atoms, cyano group, halogen atom, aromatic hydrocarbon group having 6 to 30 ring carbon atoms, substituted or an unsubstituted fused aromatic hydrocarbon group having 10 to 30 ring carbon atoms, or monocyclic aromatic heterocyclic group having 2 to 30 ring carbon atoms:
Y¹, Y³ and Y⁴ independently represent a hydrogen atom, fluorine atom, cyano group, substituted or unsubstituted alkyl group having 1 to 20 carbon atoms, substituted or unsubstituted alkoxy group having 1 to 20 carbon atoms, substituted or unsubstituted haloalkyl group having 1 to 20 carbon atoms, substituted or unsubstituted haloalkoxy group having 1 to 20 carbon atoms, substituted or unsubstituted alkylsilyl having 1 to 10 carbon atoms, substituted or unsubstituted arylsilyl having 6 to 30 carbon atoms, substituted or unsubstituted aromatic hydrocarbon group having 6 to 30 ring carbon atoms, substituted or unsubstituted fused aromatic hydrocarbon group having 10 to 30 ring carbon atoms, substituted or unsubstituted aromatic heterocyclic group having 2 to 30 ring carbon atoms, or substituted or unsubstituted fused aromatic heterocyclic group having 2 to 30 ring carbon atoms:
Y² represents a hydrogen atom, fluorine atom, cyano group, unsubstituted alkyl group having 1 to 20 carbon atoms, unsubstituted alkoxy group having 1 to 20 carbon atoms, unsubstituted haloalkyl group having 1 to 20 carbon atoms, unsubstituted haloalkoxy group having 1 to 20 carbon atoms unsubstituted alkylsilyl having 1 to 10 carbon atoms, unsubstituted arylsilyl having 6 to 30 carbon atoms, unsubstituted aromatic hydrocarbon group having 6 to 30 ring carbon atoms, unsubstituted fused aromatic hydrocarbon group having 10 to 30 ring carbon atoms unsubstituted monocyclic aromatic heterocyclic group having 2 to 30 ring carbon atoms, or unsubstituted fused aromatic heterocyclic group having 2 to 30 ring carbon atoms;
adjacent ones of Y¹ to Y⁴ are allowed to be bonded to each other to form a ring structure;
p and q represent an integer of 1 to 4; r and s represent an integer of 1 to 3; and
when p and q are an integer of 2 to 4 and r and s are an integer of 2 to 3, a plurality of Y¹ to Y⁴ are allowed to be the same or different.

34. The biscarbazole derivative according to claim 33, wherein the biscarbazole derivative is represented by a formula 3 below, 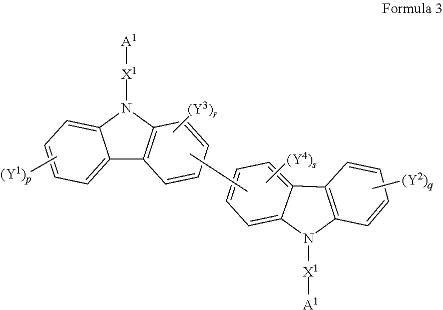
where: A¹, A², X¹, X², Y¹ to Y⁴, p, q, r and s represent the same as A¹, A², X¹, X², Y¹ to Y⁴, p, q, r and s of the formula 2.

35. The biscarbazole derivative according to claim 34, wherein in formula 3 when A¹ has a substituent, the substituent of A¹ is an aromatic hydrocarbon group having 6 to 30 ring carbon atoms or a substituted or an unsubstituted fused aromatic hydrocarbon group having 10 to 30 ring carbon atoms;
Y¹ to Y⁴ are a hydrogen atom; and
when A² has a substituent, the substituent of A² is an aromatic hydrocarbon group having 6 to 30 ring carbon atoms or a substituted or an unsubstituted fused aromatic hydrocarbon group having 10 to 30 ring carbon atoms.

36. The biscarbazole derivative according to claim 34, wherein in formula 3
when A¹ has a substituent, the substituent of A¹ is an aromatic hydrocarbon group having 6 to 30 ring carbon atoms or a substituted or an unsubstituted fused aromatic hydrocarbon group having 10 to 30 ring carbon atoms;
Y¹ to Y⁴ are a hydrogen atom;
when A² has a substituent, the substituent of A² is an aromatic hydrocarbon group having 6 to 30 ring carbon atoms or a substituted or an unsubstituted fused aromatic hydrocarbon group having 10 to 30 ring carbon atoms; and
X¹ is a single bond.

37. The biscarbazole derivative according to claim 34, wherein in formula 3
when A¹ has a substituent, the substituent of A¹ is an aromatic hydrocarbon group having 6 to 30 ring carbon atoms or a substituted or an unsubstituted fused aromatic hydrocarbon group having 10 to 30 ring carbon atoms;
Y¹ to Y⁴ are a hydrogen atom;
when A² has a substituent, the substituent of A² is an aromatic hydrocarbon group having 6 to 30 ring carbon atoms or a substituted or an unsubstituted fused aromatic hydrocarbon group having 10 to 30 ring carbon atoms; and
X¹ is an unsubstituted aromatic hydrocarbon group having 6 to 30 ring carbon atoms or an unsubstituted fused aromatic hydrocarbon group having 10 to 30 ring carbon atoms.

38. An organic-EL-device material comprising the biscarbazole derivative according to claim 33.

39. An organic electroluminescence device comprising: a cathode: an anode; and a plurality of organic thin-film layers provided between the cathode and the anode, the organic thin-film layers comprising an emitting layer, wherein
at least one of the organic thin-film layers comprises the organic-EL-device material according to claim 38.

40. The organic electroluminescence device according to claim 39, wherein the emitting layer comprises the organic-EL-device material as a host material.

41. The organic electroluminescence device according to claim 39, wherein the emitting layer comprises a phosphorescent material.

42. The organic electroluminescence device according to claim 41, wherein the phosphorescent material is an ortho-metalated complex of a metal atom selected from iridium (Ir), osmium (Os) and platinum (Pt).

43. The organic electroluminescence device according to claim 39, wherein an electron injecting layer is provided between the cathode and the emitting layer, the electron injecting layer comprising a nitrogen-containing cyclic derivative.

44. The organic electroluminescence device according to claim 39, wherein an electron transporting layer is provided between the cathode and the emitting layer, the electron transporting layer comprising the organic-EL-device material.

45. The organic electroluminescence device according to claim 39, wherein a reduction-causing dopant is present at an interfacial region between the cathode and at least one of the organic thin-film layers.

46. An organic electroluminescence device comprising: a cathode; an anode; and a plurality of organic thin-film layers provided between the cathode and the anode, the organic thin-film layers comprising an emitting layer, wherein
at least one of the organic thin-film layers is the emitting layer comprising a first host material, a second host material and a phosphorescent material providing phosphorescence,
the first host material being a compound represented by a formula (4) below, 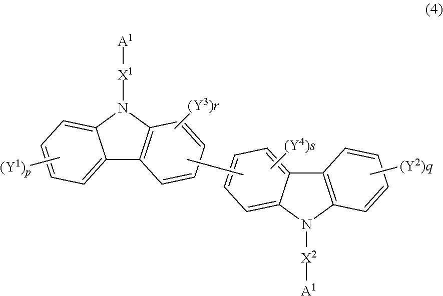
where: A¹ represents a substituted or unsubstituted quinazoline ring bound to X¹ through the nitrogen-containing ring of the quinazoline, when A¹ has a substituent, the substituent of A¹ is an alkyl group having 1 to 20 carbon atoms, alkoxy group having 1 to 20 carbon atoms, haloalkyl group having 1 to 20 carbon atoms, alkylsilyl group having 1 to 10 carbon atoms, arylsilyl group having 6 to 30 ring carbon atoms, cyano group, halogen atom, aromatic hydrocarbon group having 6 to 30 ring carbon atoms, substituted or unsubstituted fused aromatic hydrocarbon group having 10 to 30 ring carbon atoms, or monocyclic aromatic heterocyclic group having 2 to 30 ring carbon atoms;
A² represents a substituted or unsubstituted aromatic hydrocarbon group having 6 to 30 ring carbon atoms, when A² has a substituent, the substituent of A² is an alkyl group having 1 to 20 carbon atoms, alkoxy group having 1 to 20 carbon atoms, haloalkyl group having 1 to 20 carbon atoms, alkylsilyl group having 1 to 10 carbon atoms, arylsilyl group having 6 to 30 ring carbon atoms, cyano group, halogen atom, aromatic hydrocarbon group having 6 to 30 ring carbon atoms, substituted or an unsubstituted fused aromatic hydrocarbon group having 10 to 30 ring carbon atoms, or monocyclic aromatic heterocyclic group having 2 to 30 ring carbon atoms;
X¹ and X² each are a linking group and independently represent a single bond, substituted or unsubstituted aromatic hydrocarbon group having 6 to 30 ring carbon atoms, substituted or unsubstituted fused aromatic hydrocarbon group having 10 to 30 ring carbon atoms, when X¹ and X² each have a substituent, the substituent of X¹ and X² is an alkyl group having 1 to 20 carbon atoms, alkoxy group having 1 to 20 carbon atoms, haloalkyl group having 1 to 20 carbon atoms, alkylsilyl group having 1 to 10 carbon atoms, arylsilyl group having 6 to 30 ring carbon atoms, cyano group, halogen atom, aromatic hydrocarbon group having 6 to 30 ring carbon atoms, substituted or an unsubstituted fused aromatic hydrocarbon group having 10 to 30 ring carbon atoms, or monocyclic aromatic heterocyclic group having 2 to 30 ring carbon atoms;
Y¹, Y³ and Y⁴ independently represent a hydrogen atom, fluorine atom, cyano group, substituted or unsubstituted alkyl group having 1 to 20 carbon atoms, substituted or unsubstituted alkoxy group having 1 to 20 carbon atoms, substituted or unsubstituted haloalkyl group having 1 to 20 carbon atoms, substituted or unsubstituted haloalkoxy group having 1 to 20 carbon atoms, substituted or unsubstituted alkylsilyl having 1 to 10 carbon atoms, substituted or unsubstituted arylsilyl having 6 to 30 carbon atoms, substituted or unsubstituted aromatic hydrocarbon group having 6 to 30 ring carbon atoms, substituted or unsubstituted fused aromatic hydrocarbon group having 10 to 30 ring carbon atoms, substituted or unsubstituted aromatic heterocyclic group having 2 to 30 ring carbon atoms, or substituted or unsubstituted fused aromatic heterocyclic group having 2 to 30 ring carbon atoms;
Y² represents a hydrogen atom, fluorine atom, cyano group, unsubstituted alkyl group having 1 to 20 carbon atoms, unsubstituted alkoxy group having 1 to 20 carbon atoms, unsubstituted haloalkyl group having 1 to 20 carbon atoms, unsubstituted haloalkoxy group having 1 to 20 carbon atoms, unsubstituted alkylsilyl having 1 to 10 carbon atoms, unsubstituted arylsilyl having 6 to 30 carbon atoms, unsubstituted aromatic hydrocarbon group having 6 to 30 ring carbon atoms, unsubstituted fused aromatic hydrocarbon group having 10 to 30 ring carbon atoms, unsubstituted monocyclic aromatic heterocyclic group having 2 to 30 ring carbon atoms, or unsubstituted fused aromatic heterocyclic group having 2 to 30 ring carbon atoms;
adjacent ones of Y¹ to Y¹ are allowed to be bonded to each other to form a ring structure:
p and q represent an integer of 1 to 4; r and s represent an integer of 1 to 3; and
when p and q are an integer of 2 to 4 and r and s are an integer of 2 to 3, a plurality of Y¹ to Y⁴ are allowed to be the same or different.

47. The organic electroluminescence device according to claim 46, wherein the second host material is represented by either one of a formula (13) or (14) below, 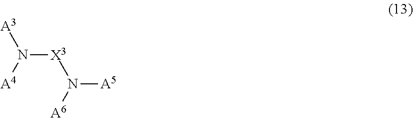
where: X³ represents a substituted or unsubstituted arylene group having 10 to 40 ring carbon atoms; and
A³ to A⁶ represent a substituted or unsubstituted aryl group having 6 to 60 ring carbon atoms, or heteroaryl group having 6 to 60 ring atoms, 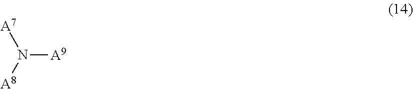
where: A⁷ to A⁹ represent a substituted or unsubstituted aryl group having 6 to 60 ring carbon atoms, or heteroaryl group having 6 to 60 ring atoms.

48. The organic electroluminescence device according to claim 47, wherein the second host material is represented by any one of formulae (15) to (19) below, 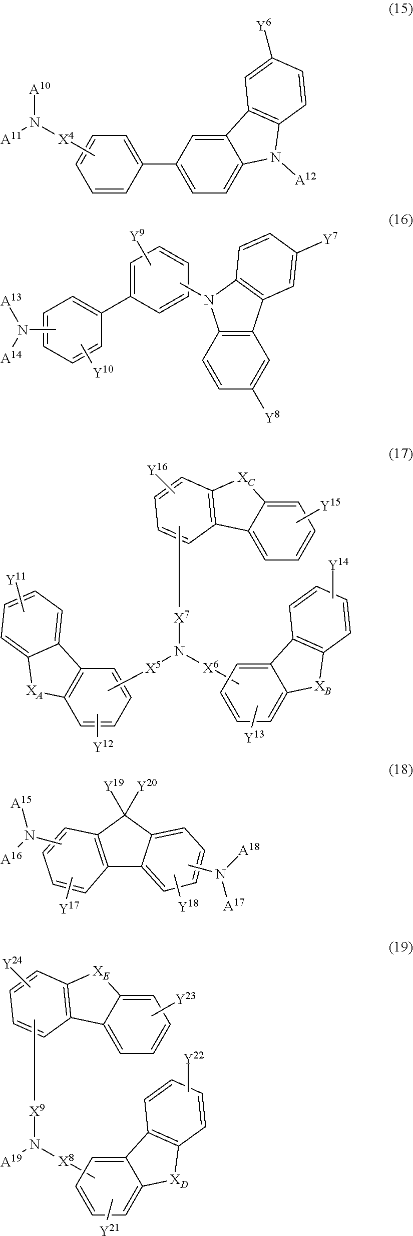
where: A¹⁰ to A¹⁹ each represent a substituted or unsubstituted aryl group having 6 to 40 carbon atoms, or substituted or unsubstituted aromatic heterocyclic group having 2 to 40 carbon atoms;
variable pairs A¹⁰ and A¹; A¹³ and A¹⁴; A¹⁵ and A¹⁶; A¹⁷ and A¹⁸ together with the nitrogen to which they are bonded optionally form a ring;
X⁴ to X⁹ represent a single bond or a linking group having 1 to 30 carbon atoms;
Y⁶ to Y²⁴ represent a hydrogen atom, halogen atom, substituted or unsubstituted alkyl group having 1 to 40 carbon atoms, substituted or unsubstituted heterocyclic group having 3 to 20 carbon atoms, substituted or unsubstituted aryl group having 6 to 40 carbon atoms, substituted or unsubstituted aralkyl group having 7 to 20 carbon atoms, substituted or unsubstituted alkenyl group having 2 to 40 carbon atoms, substituted or unsubstituted alkylamino group having 1 to 40 carbon atoms, substituted or unsubstituted aralkylamino group having 7 to 60 carbon atoms, substituted or unsubstituted alkylsilyl group having 3 to 20 carbon atoms, substituted or unsubstituted arylsilyl group having 8 to 40 carbon atoms, substituted or unsubstituted aralkylsilyl group having 8 to 40 carbon atoms, or substituted or unsubstituted halogenated alkyl group having 1 to 40 carbon atoms; and
X_{A}, X_{B}, X_{C}, X_{D}, X_{E} each represent a sulfur atom, an oxygen atom or a monoaryl-substituted nitrogen atom.

49. The organic electroluminescence device according to claim 46, wherein the emitting layer comprises a host material and a phosphorescent material, the phosphorescent material being an ortho-metalated complex of a metal atom selected from iridium (Ir), osmium (Os) and platinum (Pt).

50. The organic electroluminescence device according to claim 46, wherein an electron injecting layer is provided between the cathode and the emitting layer, the electron injecting layer comprising a nitrogen-containing cyclic derivative.

51. The organic electroluminescence device according to claim 46, wherein an electron transporting layer is provided between the cathode and the emitting layer, the electron transporting layer comprising a compound represented by the formula (4).

52. The organic electroluminescence device according to claim 46, wherein a reduction-causing dopant is present at an interfacial region between the cathode and at least one of the organic thin-film layers.

53. The biscarbazole derivative according to claim 33, wherein in formula 2 when A¹ has a substituent, the substituent of A¹ is an aromatic hydrocarbon group having 6 to 30 ring carbon atoms or a substituted or an unsubstituted fused aromatic hydrocarbon group having 10 to 30 ring carbon atoms;
Y¹ to Y⁴ are a hydrogen atom; and
when A² has a substituent, the substituent of A² is an aromatic hydrocarbon group having 6 to 30 ring carbon atoms or a substituted or an unsubstituted fused aromatic hydrocarbon group having 10 to 30 ring carbon atoms.

54. The biscarbazole derivative according to claim 33, wherein in formula 2 when A¹ has a substituent, the substituent of A¹ is an aromatic hydrocarbon group having 6 to 30 ring carbon atoms or a substituted or an unsubstituted fused aromatic hydrocarbon group having 10 to 30 ring carbon atoms;
Y¹ to Y⁴ are a hydrogen atom;
when A² has a substituent, the substituent of A² is an aromatic hydrocarbon group having 6 to 30 ring carbon atoms or a substituted or an unsubstituted fused aromatic hydrocarbon group having 10 to 30 ring carbon atoms; and
X¹ is a single bond.

55. The biscarbazole derivative according to claim 33, wherein in formula 2
when A¹ has a substituent, the substituent of A¹ is an aromatic hydrocarbon group having 6 to 30 ring carbon atoms or a substituted or an unsubstituted fused aromatic hydrocarbon group having 10 to 30 ring carbon atoms;
Y¹ to Y⁴ are a hydrogen atom;
when A² has a substituent, the substituent of A¹ is an aromatic hydrocarbon group having 6 to 30 ring carbon atoms or a substituted or an unsubstituted fused aromatic hydrocarbon group having 10 to 30 ring carbon atoms; and
X¹ is an unsubstituted aromatic hydrocarbon group having 6 to 30 ring carbon atoms or an unsubstituted fused aromatic hydrocarbon group having 10 to 30 ring carbon atoms.

56. An organic electroluminescence device comprising: a cathode; an anode; and a plurality of organic thin-film layers provided between the cathode and the anode, the organic thin-film layers comprising an emitting layer, wherein
the emitting layer comprises the biscarbazole derivative according to claim 33 and a phosphorescent material, wherein the biscarbazole derivative is represented by formula 2, and when A¹ has a substituent, the substituent of A¹ is an aromatic hydrocarbon group having 6 to 30 ring carbon atoms or a substituted or an unsubstituted fused aromatic hydrocarbon group having 10 to 30 ring carbon atoms;
Y¹ to Y⁴ are a hydrogen atom;
when A² has a substituent, the substituent of A² is an aromatic hydrocarbon group having 6 to 30 ring carbon atoms or a substituted or an unsubstituted fused aromatic hydrocarbon group having 10 to 30 ring carbon atoms;
X¹ is a single bond, an unsubstituted aromatic hydrocarbon group having 6 to 30 ring carbon atoms or an unsubstituted fused aromatic hydrocarbon group having 10 to 30 ring carbon atoms; and
the phosphorescent material is an Ir complex.

57. The organic electroluminescence device according to claim 56, wherein X¹ is a single bond in formula 2.

58. The organic electroluminescence device according to claim 56, wherein X¹ is an unsubstituted aromatic hydrocarbon group having 6 to 30 ring carbon atoms or an unsubstituted fused aromatic hydrocarbon group having 10 to 30 ring carbon atoms in formula 2.

59. An organic electroluminescence device comprising: a cathode; an anode; and a plurality of organic thin-film layers provided between the cathode and the anode, the organic thin-film layers comprising an emitting layer, wherein
the emitting layer comprises the biscarbazole derivative according to claim 34 and a phosphorescent material,
the biscarbazole derivative being represented by the formula 3, and when A¹ has a substituent, the substituent of A¹ is an aromatic hydrocarbon group having 6 to 30 ring carbon atoms or a substituted or an unsubstituted fused aromatic hydrocarbon group having 10 to 30 ring carbon atoms;
Y¹ to Y⁴ are a hydrogen atom;
when A² has a substituent, the substituent of A² is an aromatic hydrocarbon group having 6 to 30 ring carbon atoms Or a substituted or an unsubstituted fused aromatic hydrocarbon group having 10 to 30 ring carbon atoms;
X¹ is a single bond, an unsubstituted aromatic hydrocarbon group having 6 to 30 ring carbon atoms or an unsubstituted fused aromatic hydrocarbon group having 10 to 30 ring carbon atoms; and
the phosphorescent material is an Ir complex.

60. The organic electroluminescence device according to claim 59, wherein
X¹ is a single bond in formula 3.

61. The organic electroluminescence device according to claim 59, wherein
X¹ is an unsubstituted aromatic hydrocarbon group having 6 to 30 ring carbon atoms or an unsubstituted fused aromatic hydrocarbon group having 10 to 30 ring carbon atoms in formula 3.
